(19) United States
(12) Patent Application Publication
(10) Pub. No.: US 2025/0137046 A1
(43) Pub. Date: May 1, 2025

(54) PORE

(71) Applicant: Oxford Nanopore Technologies PLC, Oxford (GB)

(72) Inventors: Elizabeth Jayne Wallace, Oxford (GB); Michael Robert Jordan, Oxford (GB); Valdemar Aksionov, Oxford (GB)

(73) Assignee: Oxford Nanopore Technologies PLC, Oxford (GB)

(21) Appl. No.: 18/722,764

(22) PCT Filed: Dec. 22, 2022
(86) PCT No.: PCT/EP2022/087410

§ 371 Date: Jun. 21, 2024

(30) Foreign Application Priority Data
Dec. 23, 2021 (GB) 2118939.4

(51) Int. Cl. C12Q 1/6869; C07K 14/195

(52) U.S. Cl. CPC C12Q 1/6869; C07K 14/195

(57) ABSTRACT

The present invention relates to novel Rhodococcus porin monomers and pores comprising the monomers, and methods of characterising analytes, such as polypeptides and polynucleotides, using the pores.

### RELATED APPLICATIONS

This application is a national stage filing under 35 U.S.C. § 371 of international application number PCT/EP2022/087410, filed Dec. 22, 2022, which claims the benefit of United Kingdom application number GB 2118939.4, filed Dec. 23, 2021, each of which is herein incorporated by reference in its entirety.

### REFERENCE TO AN ELECTRONIC SEQUENCE LISTING

The contents of the electronic sequence listing (O036670152US00-SEQ-KZM.xml; Size: 8,892 bytes; and Date of Creation: May 13, 2024) is herein incorporated by reference in its entirety.

### FIELD

The present invention relates to novel Rhodococcus porin monomers, pores comprising the monomers and methods of characterising analytes, such as polynucleotides and polypeptides, using the pores.

### INTRODUCTION

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Analytes can be organic and inorganic small molecules as well as various biological or synthetic macromolecules and polymers including polynucleotides, polypeptides, and polysaccharides. Nanopore sensing can reveal the identity and perform single molecule counting of the sensed analytes but can also provide information on the analyte composition such as nucleotide, amino acid, or glycan sequence, as well as the presence of base, amino acid, or glycan modifications such as methylation and acylation, phosphorylation, hydroxylation, oxidation, reduction, glycosylation, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the composing building blocks as the polymer is moved through the pore. During nanopore sensing, the narrowest part of the pore forms the reader head, the most discriminating part of the nanopore with respect to the current signatures as a function of the passing analyte.

For polynucleotide analytes, nucleotide discrimination is achieved via passage through such a mutant pore, but current signatures have been shown to be sequence dependent, and multiple nucleotides contributed to the observed current, so that the height of the channel constriction and extent of the interaction surface with the analyte affect the relationship between observed current and polynucleotide sequence. While the current range for nucleotide discrimination has been improved through mutation, a sequencing system would have higher performance if the current differences between nucleotides could be improved further. Accordingly, there is a need to identify novel ways to improve nanopore sensing features.

The cell wall channel of Rhodococcus corynebacteroides was purified following an established procedure (Riess and Benz, 2000, Biochim Biophys Acta, 20; 1509(1-2): 485-495). The protein was then subjected to Edman-degradation and sequencing. The partial sequence obtained was then used as input into a BLAST search, identifying a protein termed PorARc. A BLAST search for the homologues to the partial sequence of the Rhodococcus corynebacteroides cell wall channel, PorARc, identified two primary polypeptide sequences from Rhodococcus ruber, PorARr and PorBRr.

### SUMMARY

The invention relates to modified Rhodococcus porin monomers and pores comprising the monomers. The porin monomers and the pores comprising them have been modified to facilitate the characterisation of target analytes, especially target polynucleotides and polypeptides.

The inventors have surprisingly demonstrated that novel mutants of PorARr, PorBRr and PorARc display improved properties for estimating the characteristics of analytes, such as the sequence of polynucleotides or polypeptides. The mutants surprisingly display improved polynucleotide capture and nucleotide discrimination. In particular, the mutants surprisingly display an increased current range, which makes it easier to discriminate between different nucleotides, and a reduced variance of states (referred to as “median sd”), which increases the signal-to-noise ratio (referred to as “SNR”).

In particular, the invention provides:
a mutant Rhodococcus pore comprising a monomer which is a variant of the sequence shown in SEQ ID NO: 2, wherein the variant comprises one or more mutations at one or more of the following positions:
(a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
(b) S74, G76, E78, T80, D82, T84, Q108, G110, G112, T114, E116, and P117; and/or
(c) G87, E89, G91, S93, S94, A95, G96, A97, S99, T101, S103, and D104;
wherein the one or more mutations alter the ability of the monomer to interact with a target analyte;
a mutant Rhodococcus pore comprising a monomer which is a variant of the sequence shown in SEQ ID NO: 6, wherein the variant comprises one or more mutations between positions 9 and 200 of SEQ ID NO: 6 and wherein the one or more mutations alter the ability of the pore to interact with a target analyte;
a construct comprising two or more covalently attached Rhodococcus porin monomers;
a polynucleotide which encodes a mutant pore of the invention or a construct of the invention;
a pore comprising at least one construct of the invention;
a kit for characterising a target polynucleotide or a target polypeptide comprising (a) a pore of the invention and (b) a polynucleotide binding protein or a polypeptide handling enzyme;
an apparatus for characterising a target polynucleotide or a target polypeptide in a sample, comprising (a) a plurality of pores of the invention and (b) a plurality of polynucleotide binding proteins or a plurality of polypeptide handling enzymes;
a membrane comprising a pore of the invention;
an array comprising a plurality of membranes of the invention;
a system comprising (a) a membrane of the invention or an array of the invention, (b) means for applying a potential across the membrane(s) and (c) means for detecting electrical or optical signals across the membrane(s);
a method of determining the presence, absence or one or more characteristics of a target analyte, comprising (a) contacting the target analyte with a pore of the invention and (b) taking one or more measurements as the target analyte moves with respect to the pore and thereby determining the presence, absence or one or more characteristics of the target analyte;
a method of characterising a target polynucleotide, comprising (a) contacting the target polynucleotide with a pore of the invention and (b) taking one or more measurements as the target polynucleotide moves with respect to the pore and thereby characterising the target polynucleotide;
use of a pore of the invention to determine the presence, absence or one or more characteristics of a target analyte or target polynucleotide;
an apparatus comprising a transmembrane protein pore inserted into an in vitro membrane, wherein the transmembrane protein pore comprises at least one monomer which is a variant of the Rhodococcus porin monomer and comprising mutations at one or more of the following positions:
   (a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
   (b) S74, G76, E78, T80, D82, T84, Q108, G110, G112, T114, E116, and P117; and/or
   (c) G87, E89, G91, S93, S94, A95, G96, A97, S99, T101, S103, and D104, corresponding to the sequence shown in SEQ ID NO: 2; and
an apparatus produced by a method comprising:
   (i) obtaining a transmembrane protein pore comprising at least one Rhodococcus porin monomer, wherein the monomer comprises amino acid mutations at one or more of positions
      (a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
      (b) S74, G76, E78, T80, D82, T84, Q108, G110, G112, T114, E116, and P117; and/or
      (c) G87, E89, G91, S93, S94, A95, G96, A97, S99, T101, S103, and D104 corresponding to the sequence shown in SEQ ID NO: 2; and
   (ii) contacting the pore with an in vitro membrane such that the pore is inserted in the in vitro membrane.

### BRIEF DESCRIPTION OF THE FIGURES

FIG. 1 shows data for PorARc wild type (WT) and PorARc mutants E78S/D82S/E89N, and E78S/D82S/E89N/E116T/E125A/D165S.

FIG. 2 shows data for PorARc mutants E78S/D82S/E89R, E78R/D82S/E89R, and E78R/D82S/E89R/E116T/E125A/D165S.

FIG. 3 shows data for PorARc mutants E57R/E78R/D82S/E89R/E116T/E125A/D165S, D55S/E57S/D58S/E78R/D82S/E89R/E116T/E125A/D165S, and E78R/D82S/E89R/D104S/E116T/E122S/E125A/D165S.

FIG. 4 shows data for PorARc mutants D55S/E57S/D58S/E78R/D82S/E89R/D104S/E116T/E122S/E125A/D165S, and E57R/E78R/D82S/E89R/D104S/E116T/E122S/E125A/D165S.

FIG. 5 shows data for PorARc mutants E78R/D82S/E89G/E116T/E125A/D165S, E78R/D82S/E89A/E116T/E125A/D165S, and E78R/D82S/E89V/E116T/E125A/D165S.

FIG. 6 shows data for PorARc mutants E78R/D82S/E89L/E116T/E125A/D165S, E78R/D82S/E891/E116T/E125A/D165S, and E78R/D82S/E89F/E116T/E125A/D165S.

FIG. 7 shows data for PorARc mutants E78R/D82S/E89S/E116T/E125A/D165S, E78R/D82S/E89T/E116T/E125A/D165S, and E78R/D82S/E89Q/E116T/E125A/D165S.

FIG. 8 shows data for PorARc mutant E78R/D82S/E89K/E116T/E125A/D165S.

FIG. 9 shows data for PorARc mutants E78R/D82S/E89R/T101V/E116T/E125A/D165S, E78R/D82S/E89R/T101S/E116T/E125A/D165S, and E78R/D82S/E89R/T101N/E116T/E125A/D165S.

FIG. 10 shows data for PorARc mutants E78R/D82S/E89R/T101Q/E116T/E125A/D165S, and E78R/D82S/E89R/T101R/E116T/E125A/D165S.

FIG. 11 shows data for PorARc mutants E78R/D82S/E89N/T101V/E116T/E125A/D165S, E78R/D82S/E89N/T101S/E116T/E125A/D165S, and E78R/D82S/E89N/T101N/E116T/E125A/D165S.

FIG. 12 shows data for PorARc mutants E78R/D82S/E89N/T101Q/E116T/E125A/D165S, and E78R/D82S/E89N/T101R/E116T/E125A/D165S.

FIG. 13 shows data for PorARc mutants E78R/D82S/G87S/E89R/E116T/E125A/D165S, E78R/D82S/E89R/G91S/E116T/E125A/D165S, and E78R/D82S/E89R/G91N/E116T/E125A/D165S.

FIG. 14 shows data for PorARc mutants E78R/D82S/E89R/G91N/192N/E116T/E125A/D165S, E78R/D82S/E89R/I92N/E116T/E125A/D165S, and E78R/D82S/E89R/S99N/E116T/E125A/D165S.

FIG. 15 shows data for PorARc mutants E78R/D82S/E89R/S103N/E116T/E125A/D165S, E78R/D82S/E89R/D104S/E116T/E125A/D165S, and E78R/D82S/E89R/E116T/E122S/E125A/D165S.

FIG. 16 shows data for PorARc mutants E78G/D82S/E89L/G91N/192N/E116G/E125A/D165S and E78G/D82S/E89L/E116G/E125A/D165S.

FIG. 17 shows data for PorARr mutants E182R/E184R, E89N/E91N, and E89N/E91N/E168R/E182R/E184R.

FIG. 18 shows data for PorARr mutants E89N/E91N/E96R/E99N/E113N/E123N/D130N/E182R/E184R and E89N/E91N/E96R/E99R/E113K/E123K/D130K/E182R/E184R.

FIG. 19 shows data for PorBRr wild type (WT) and PorBRr mutants E90N and E92N.

FIG. 20 shows data for PorBRr mutants E90N/E92N, E90N/E92N/E115R/E131R, and E90N/E92N/E 113R/E115R/E129R/E131R.

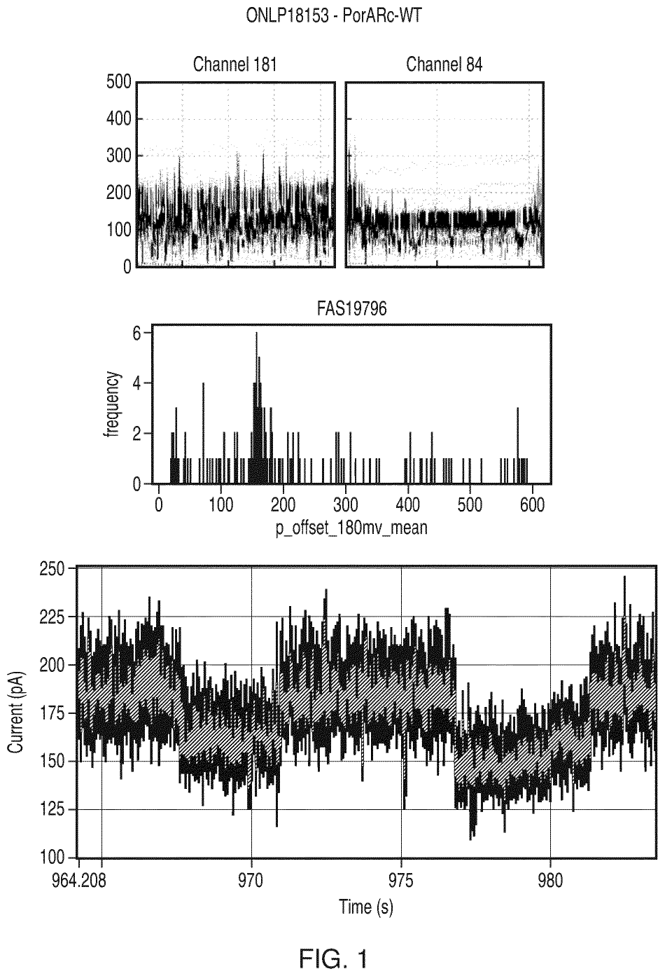

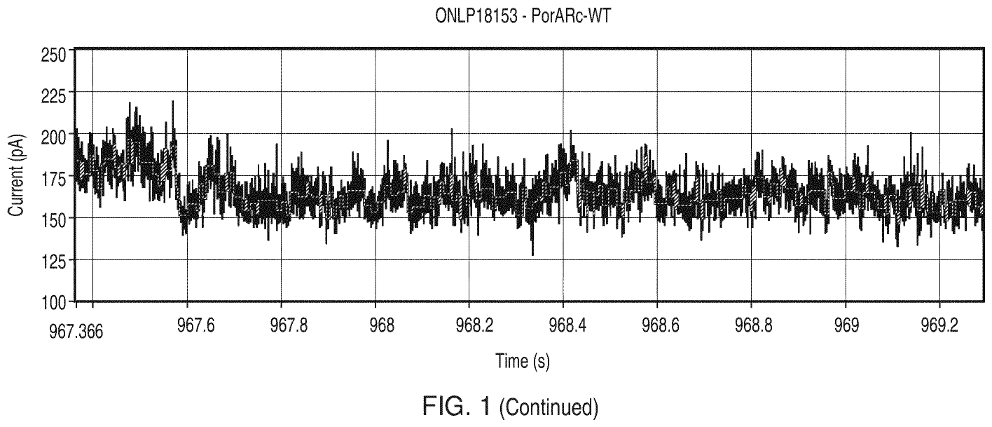

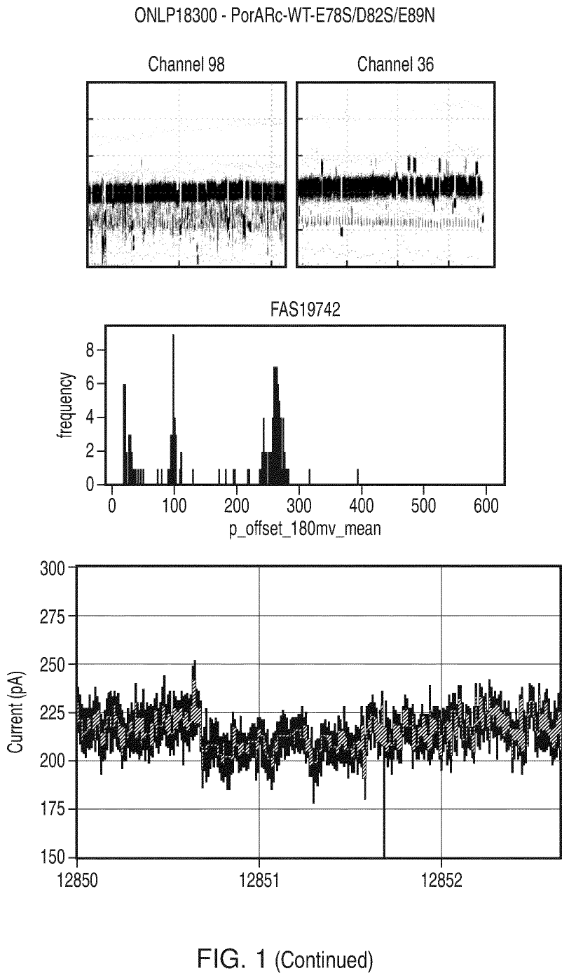

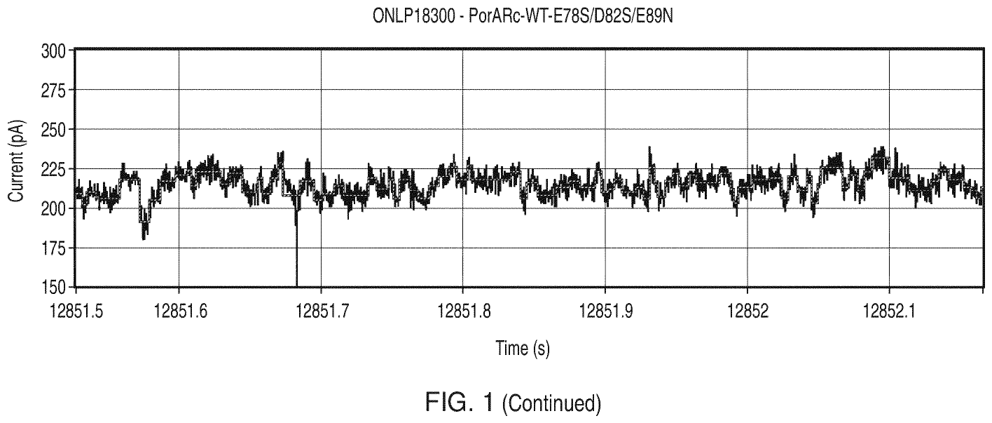

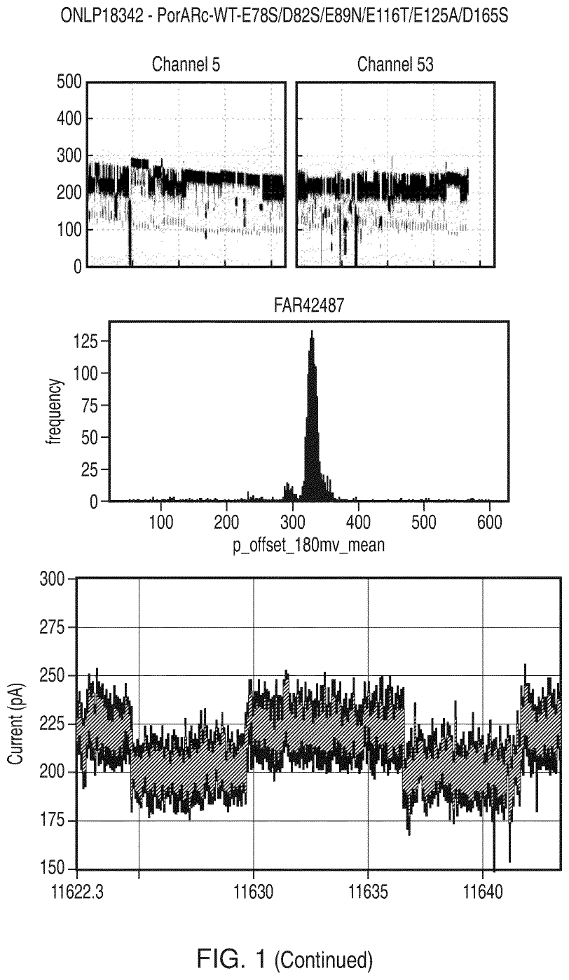

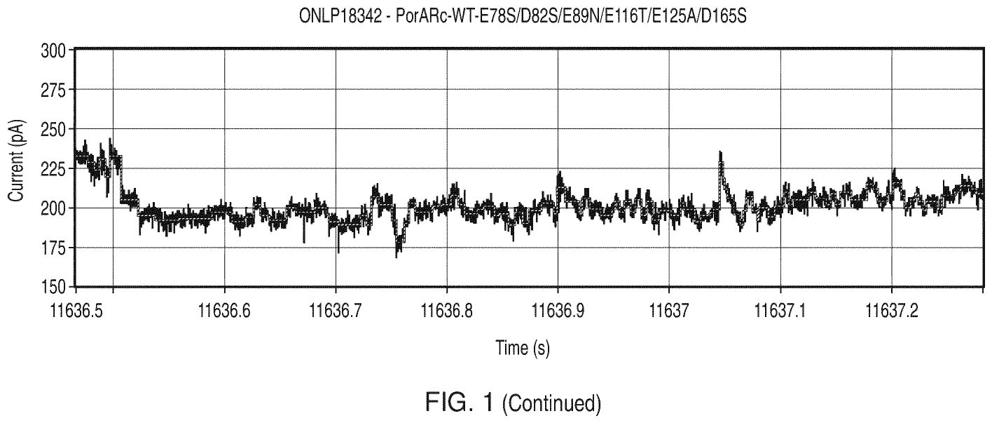

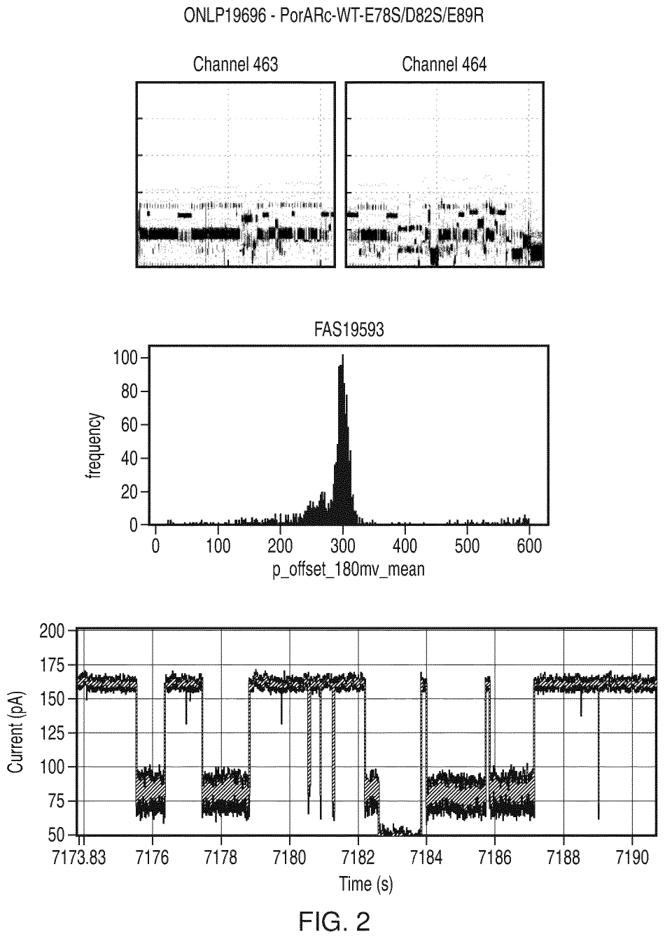

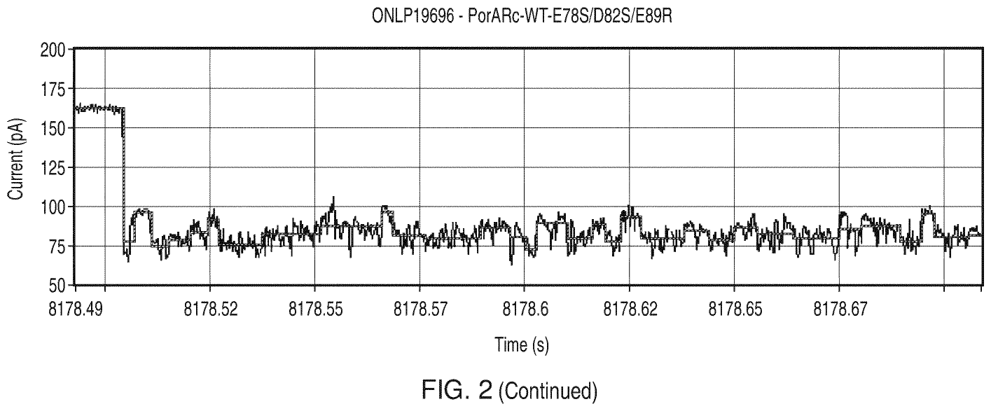

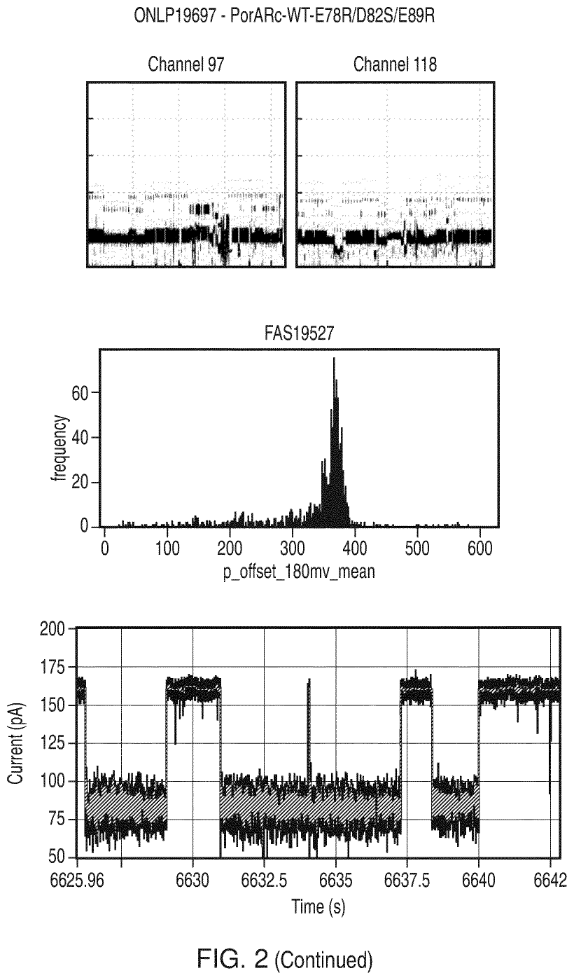

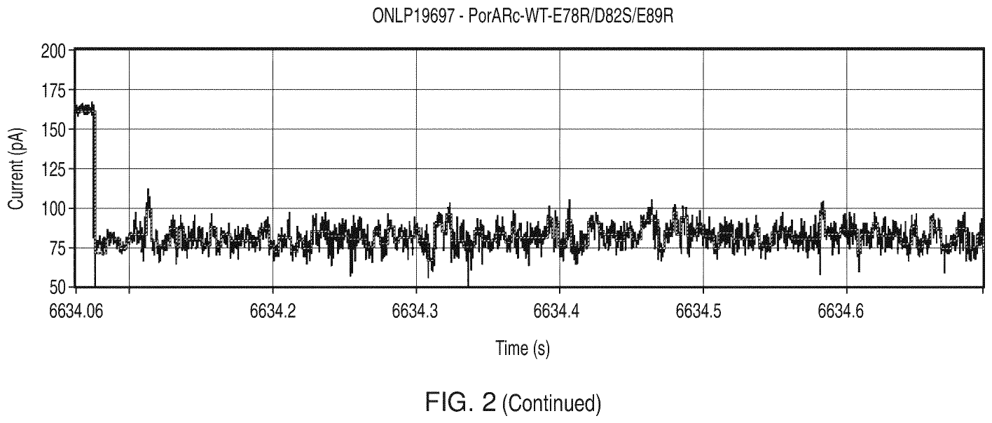

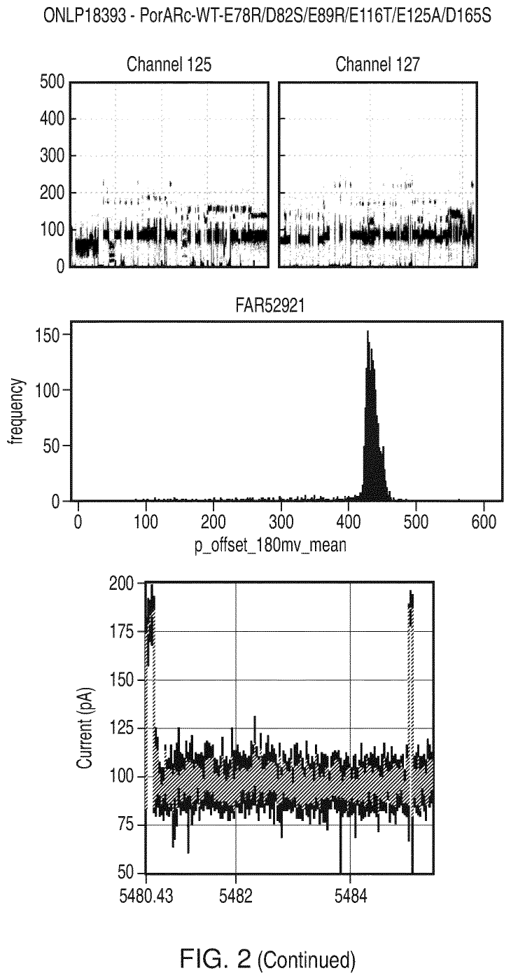

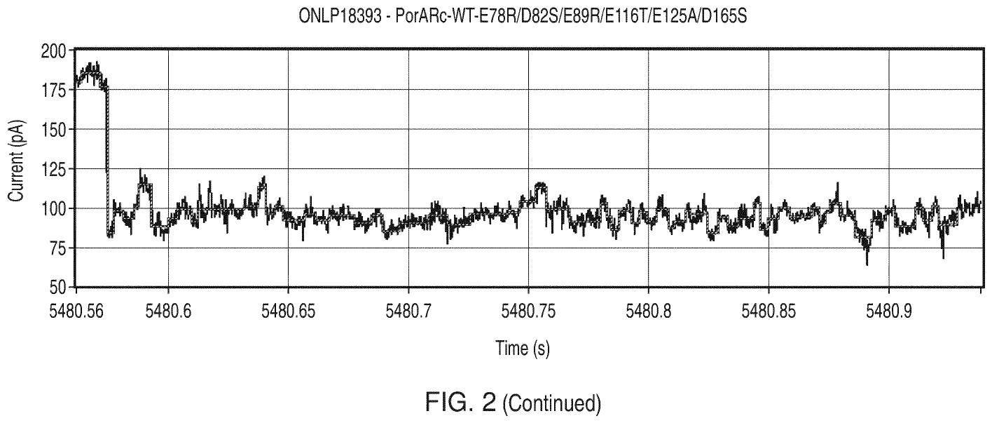

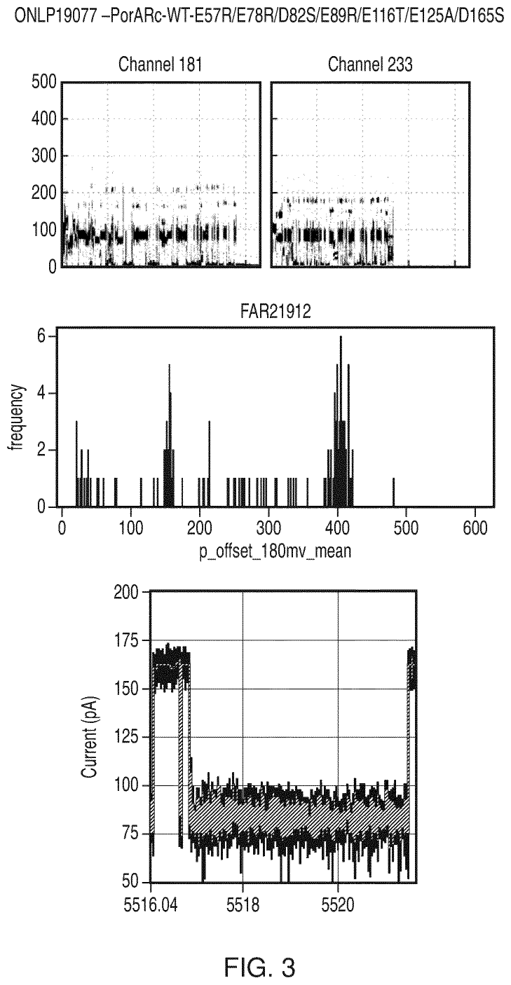

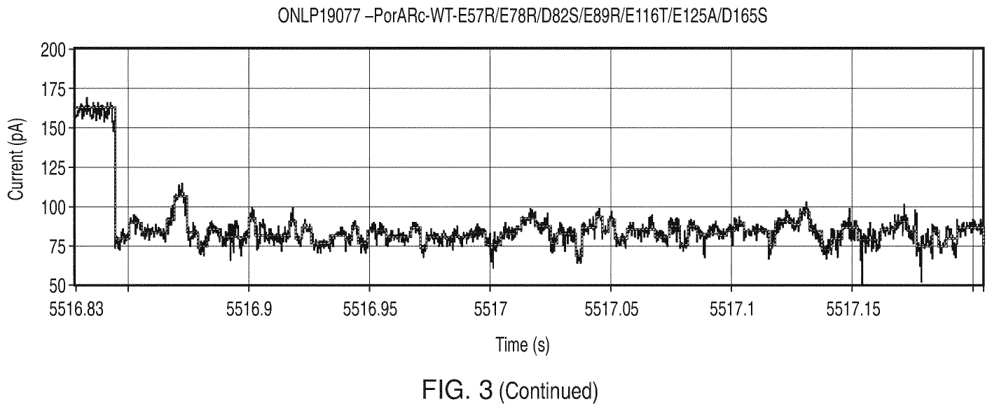

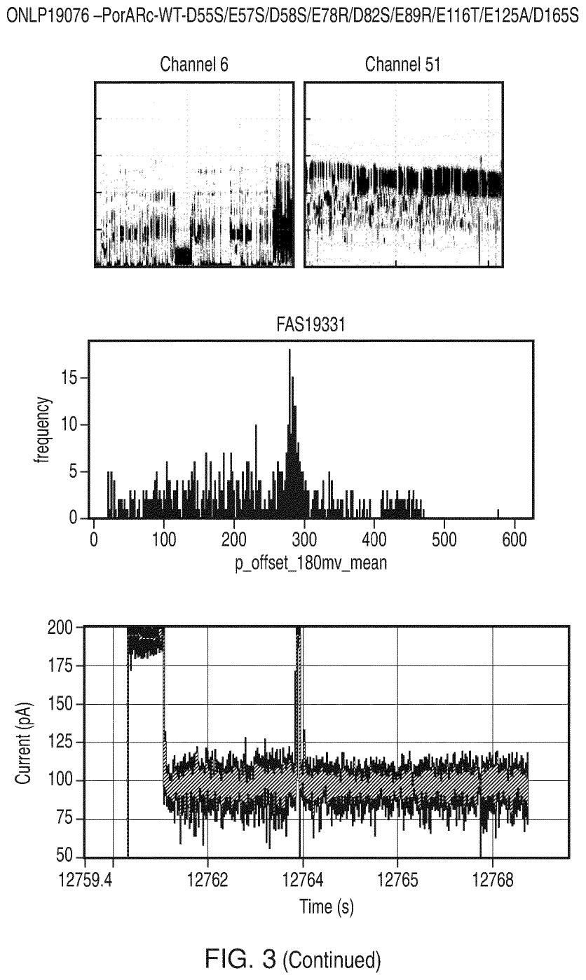

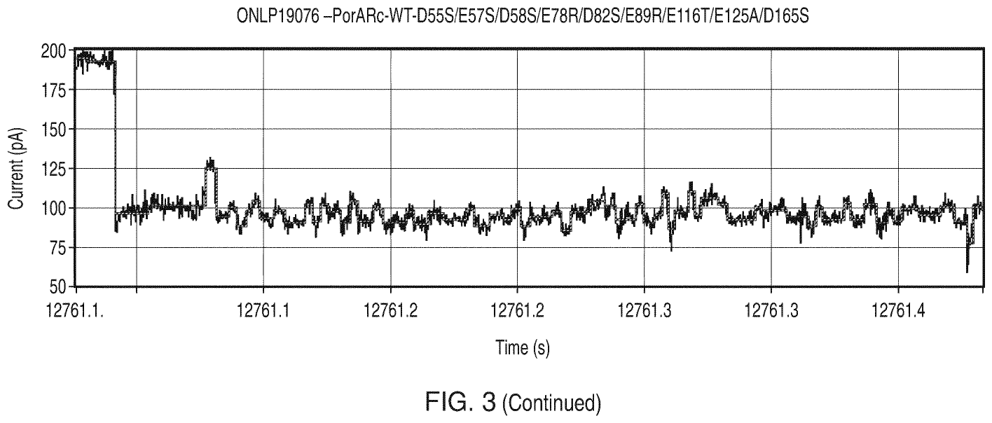

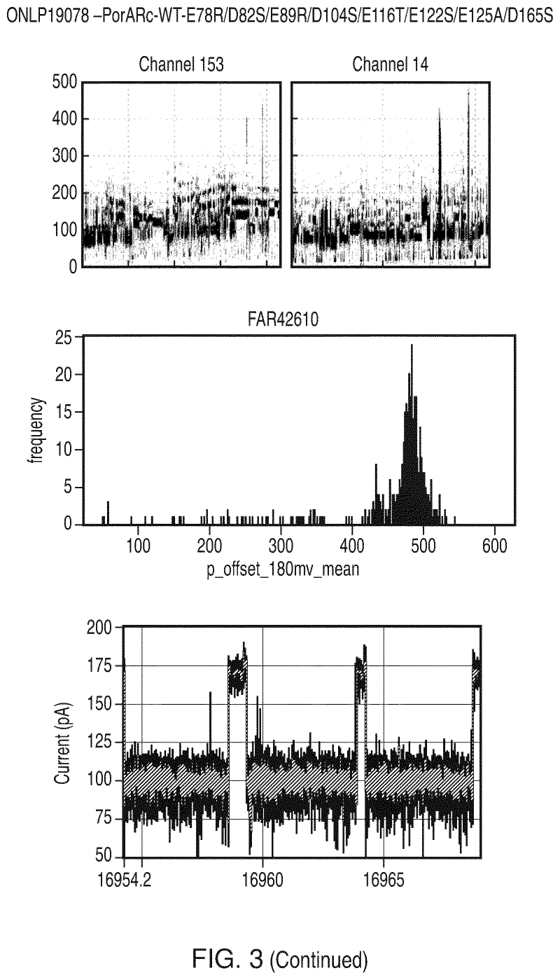

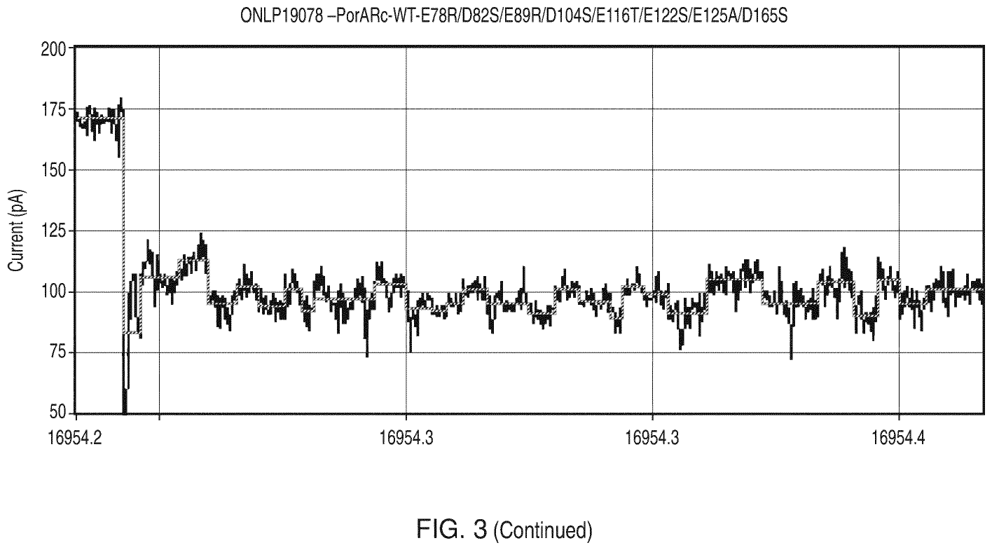

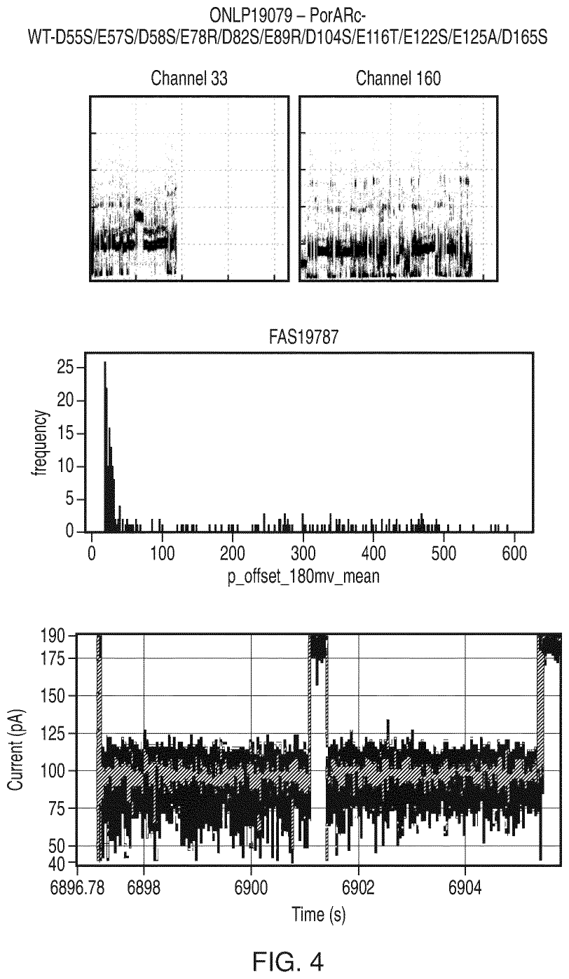

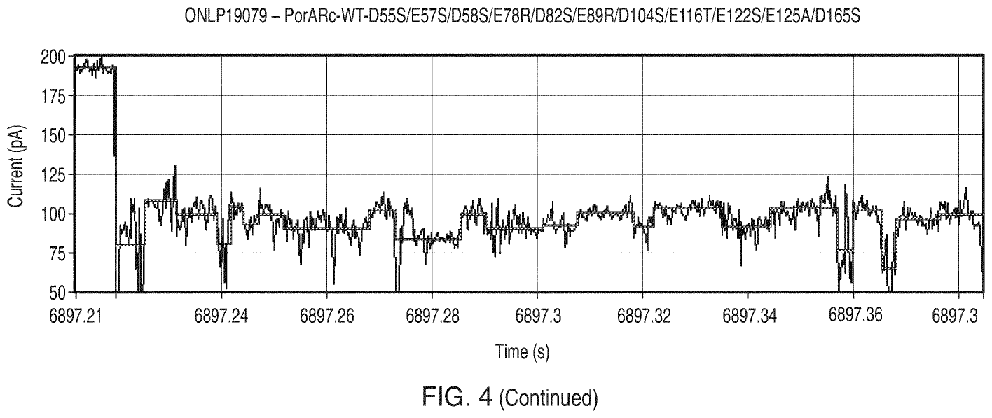

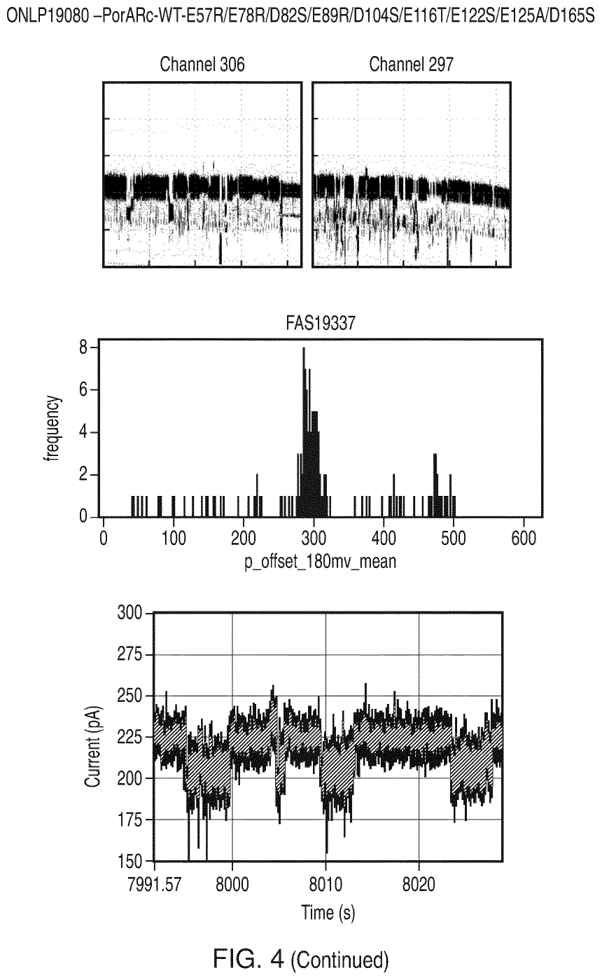

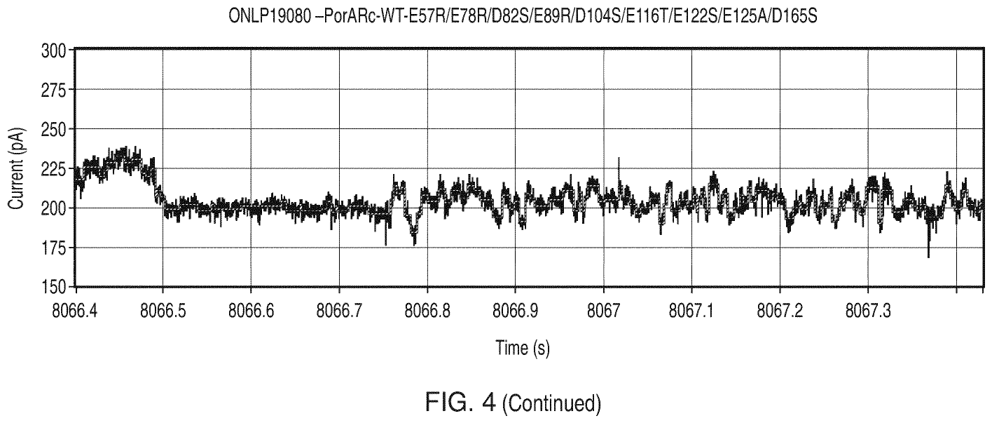

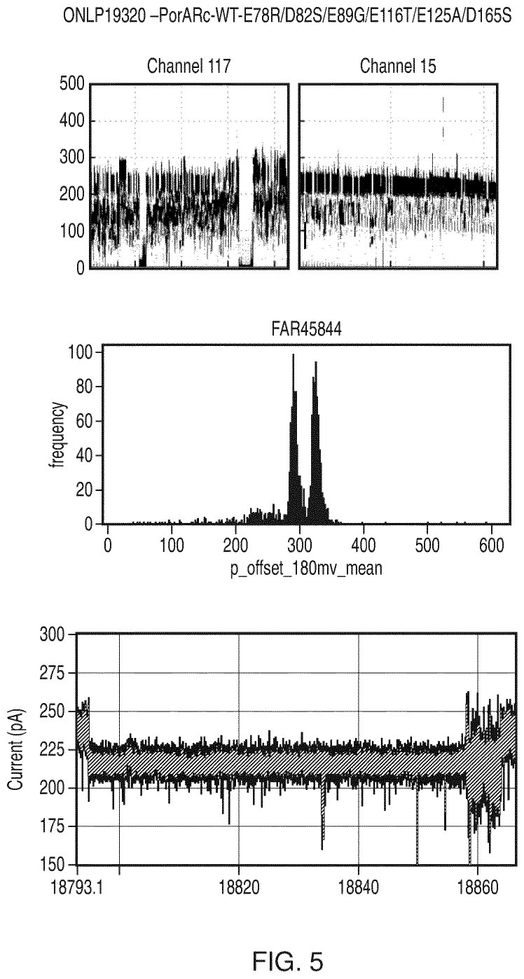

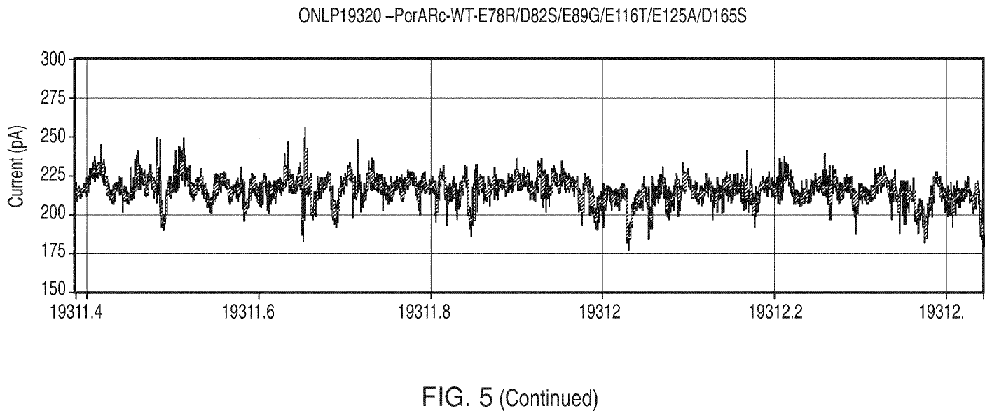

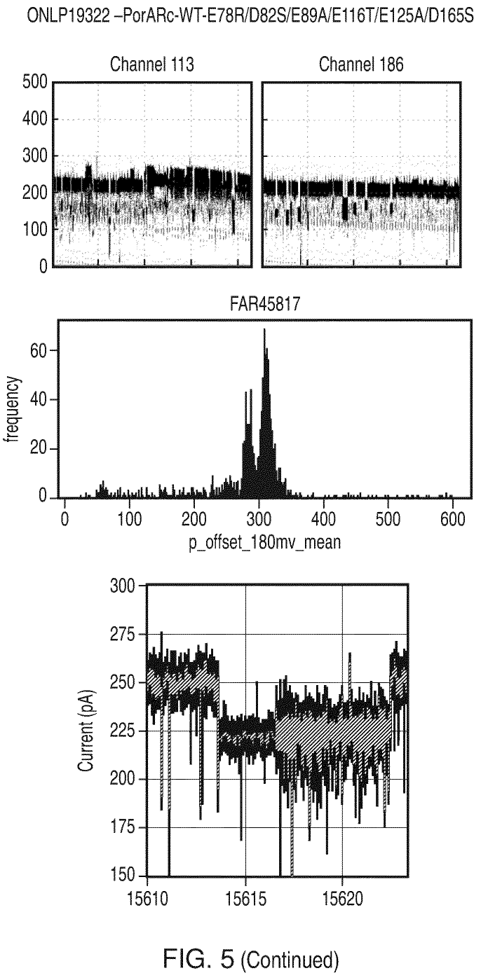

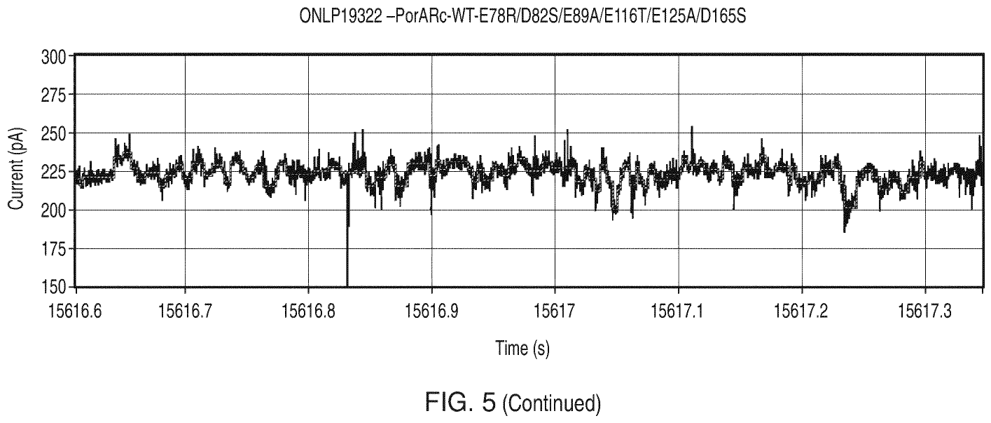

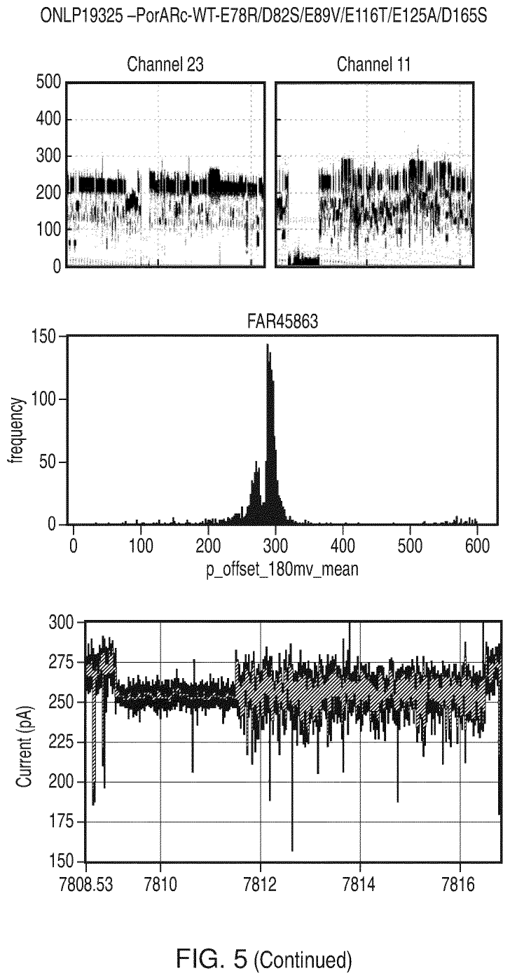

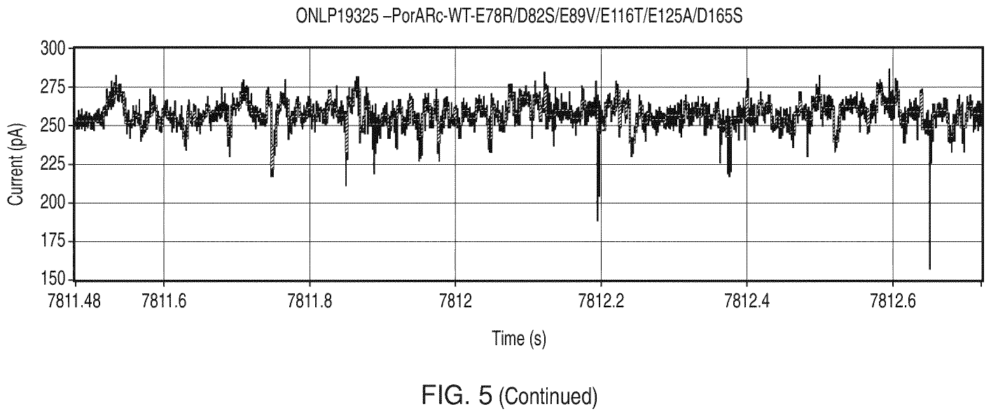

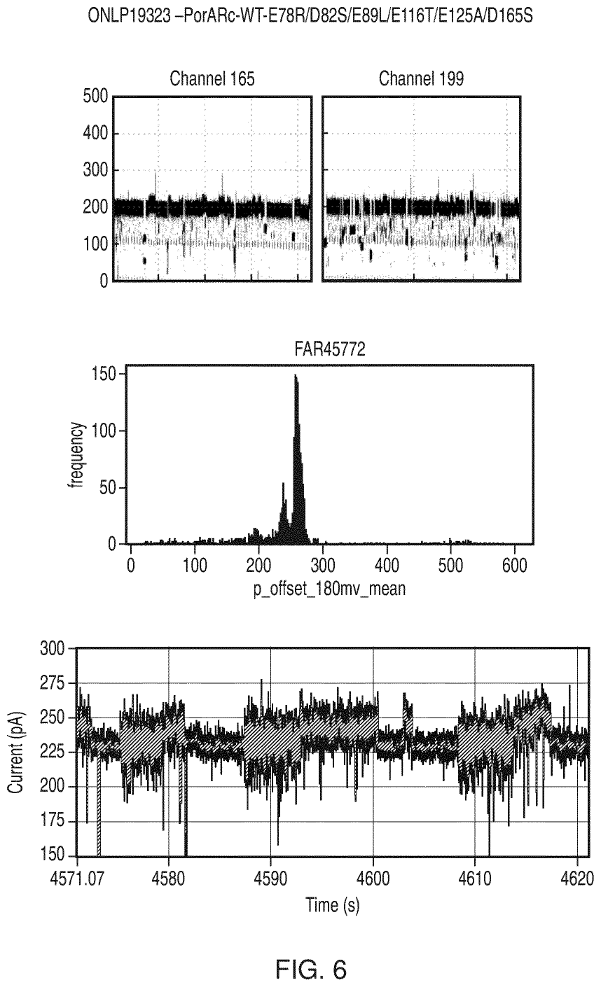

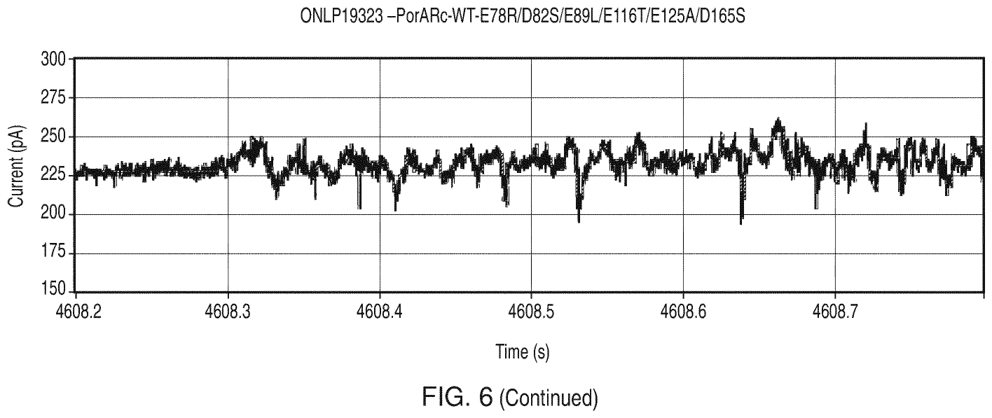

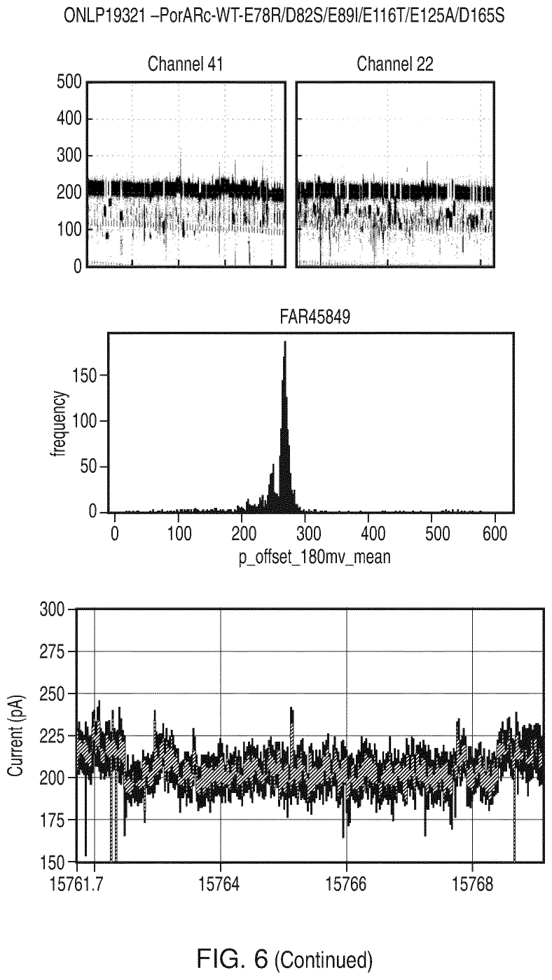

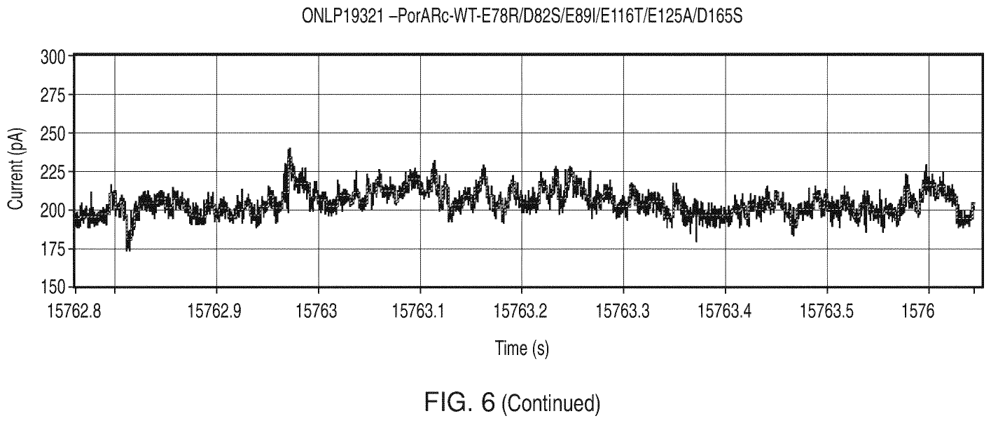

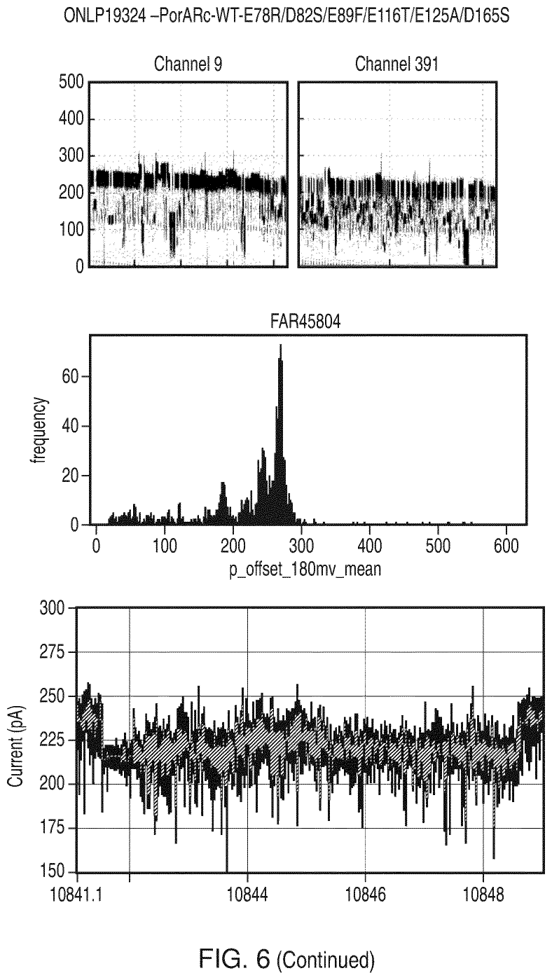

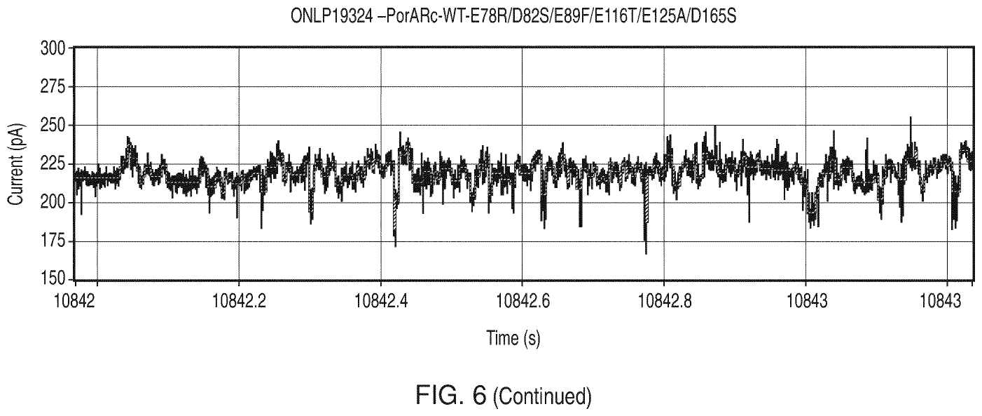

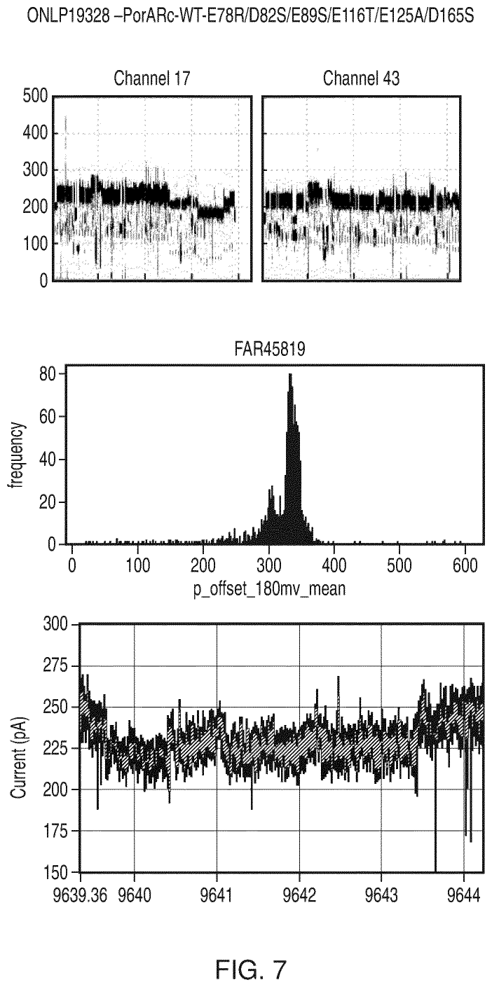

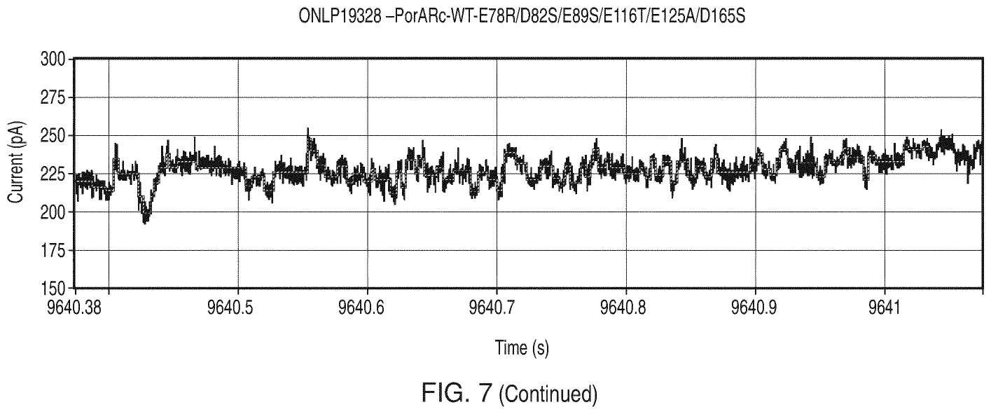

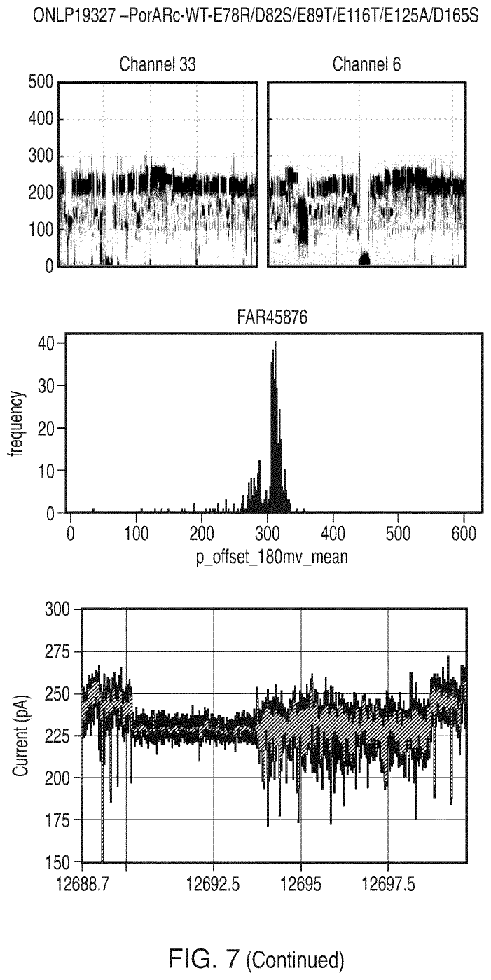

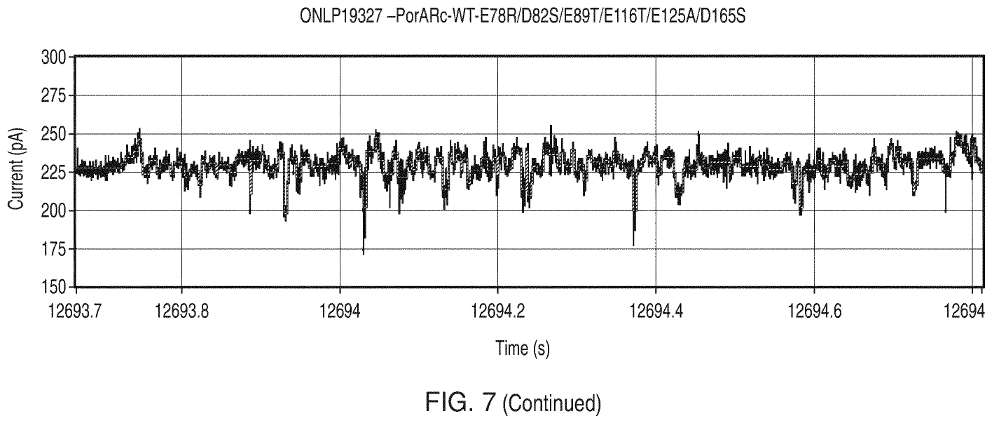

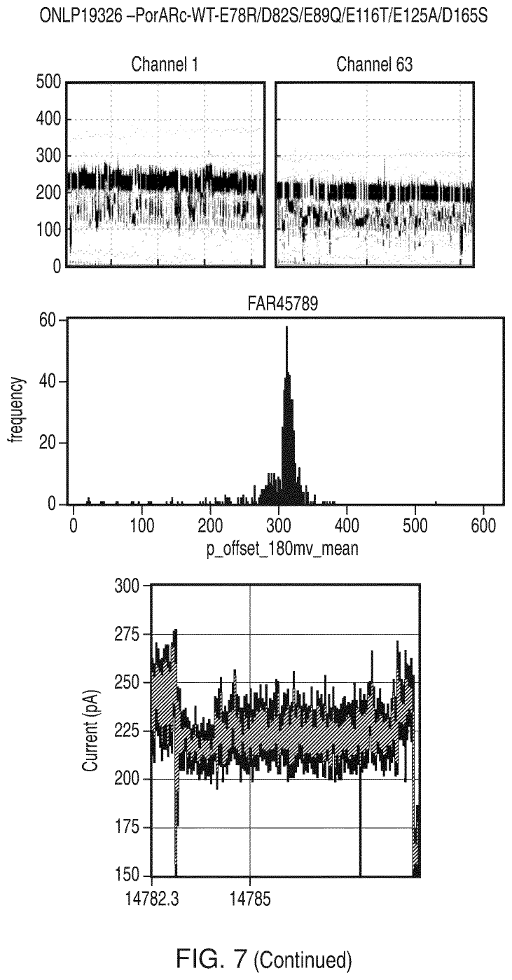

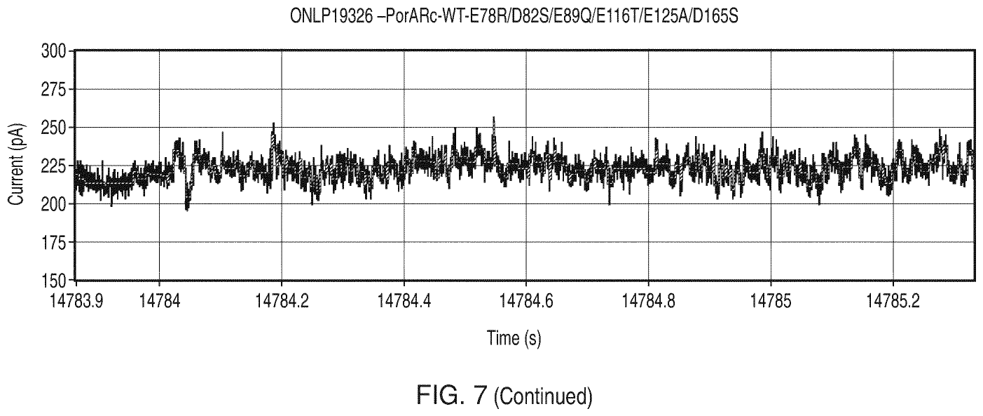

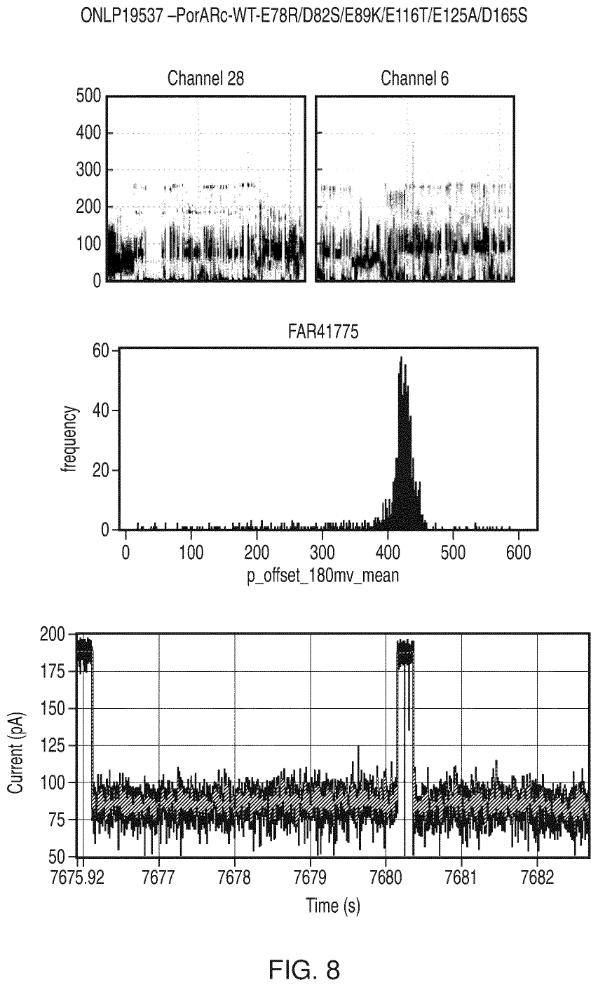

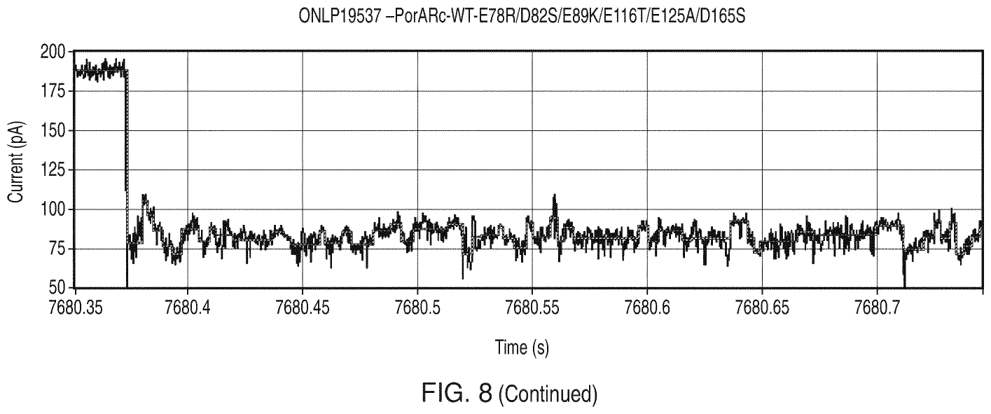

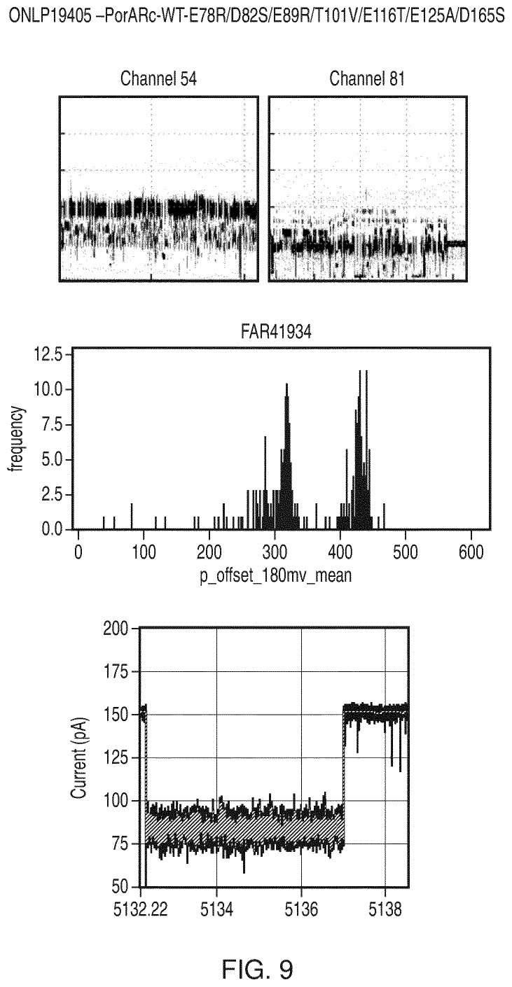

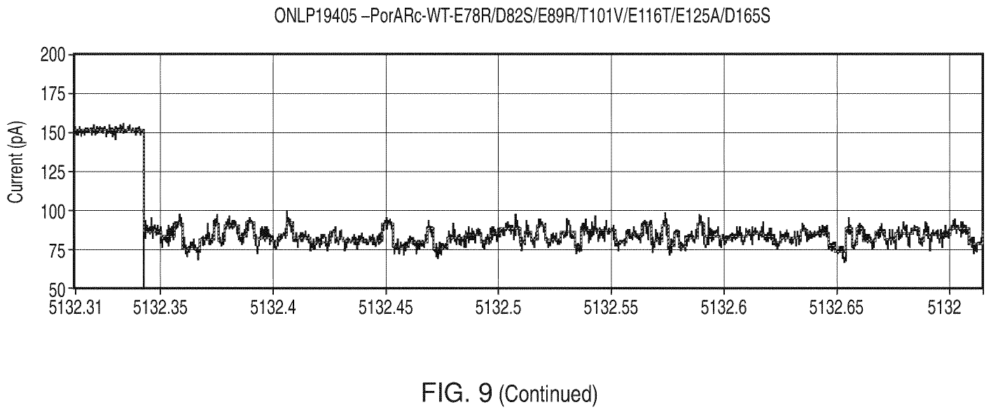

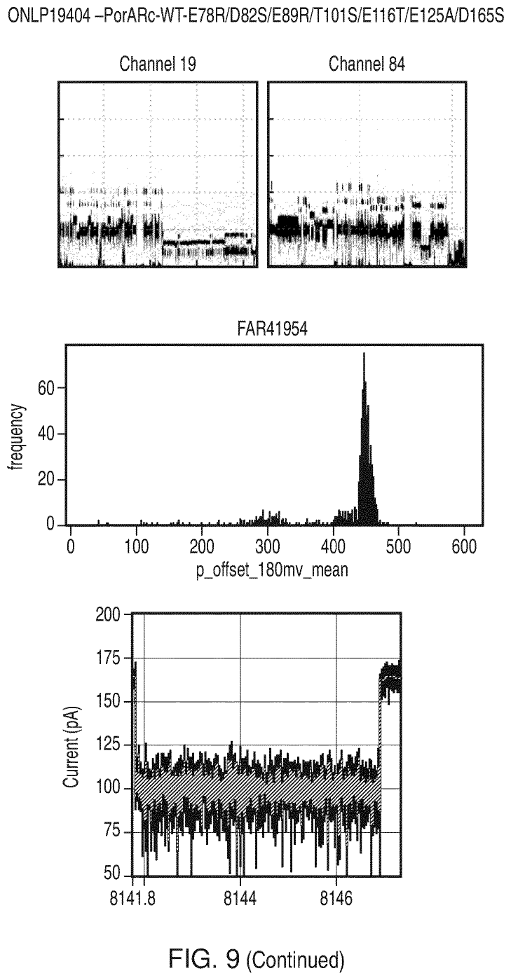

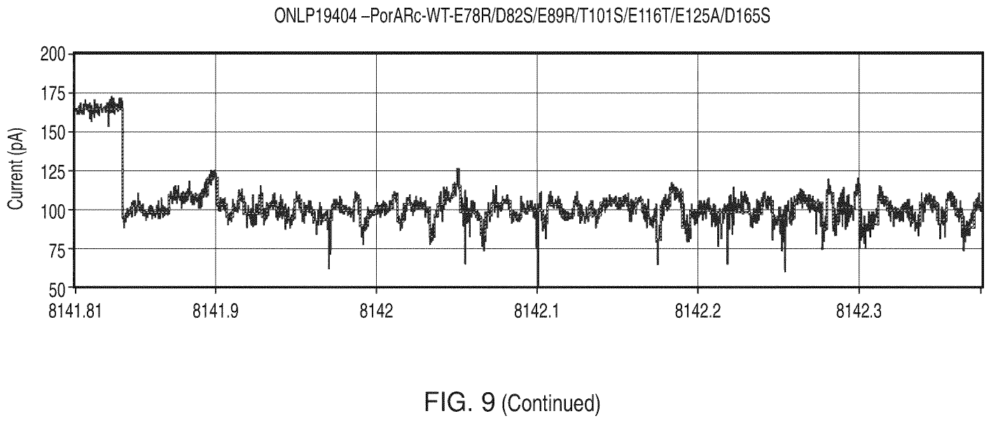

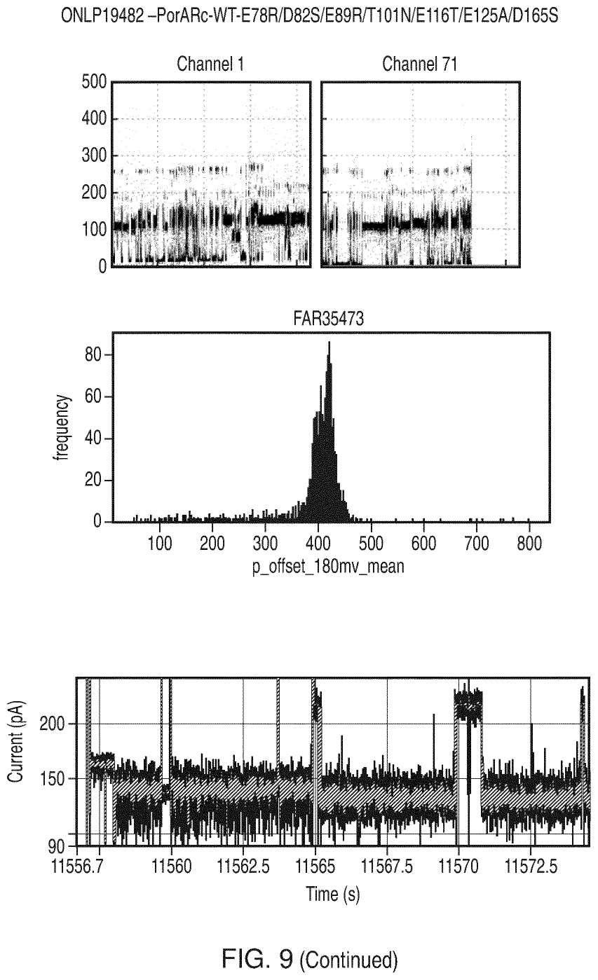

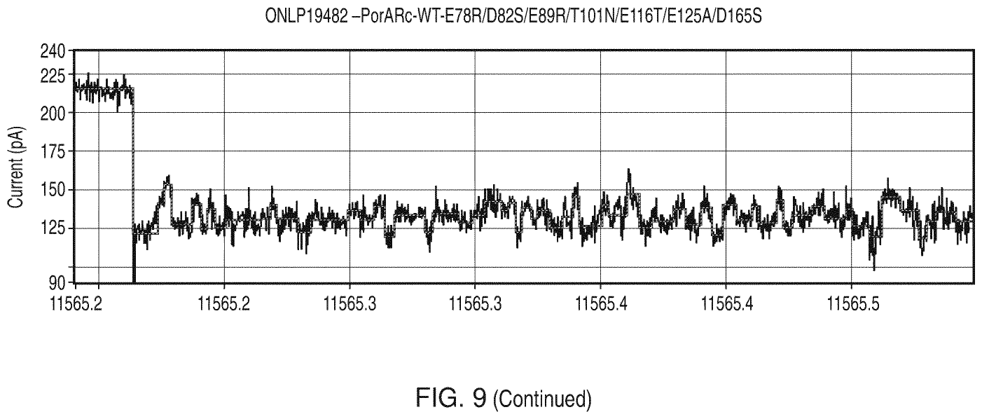

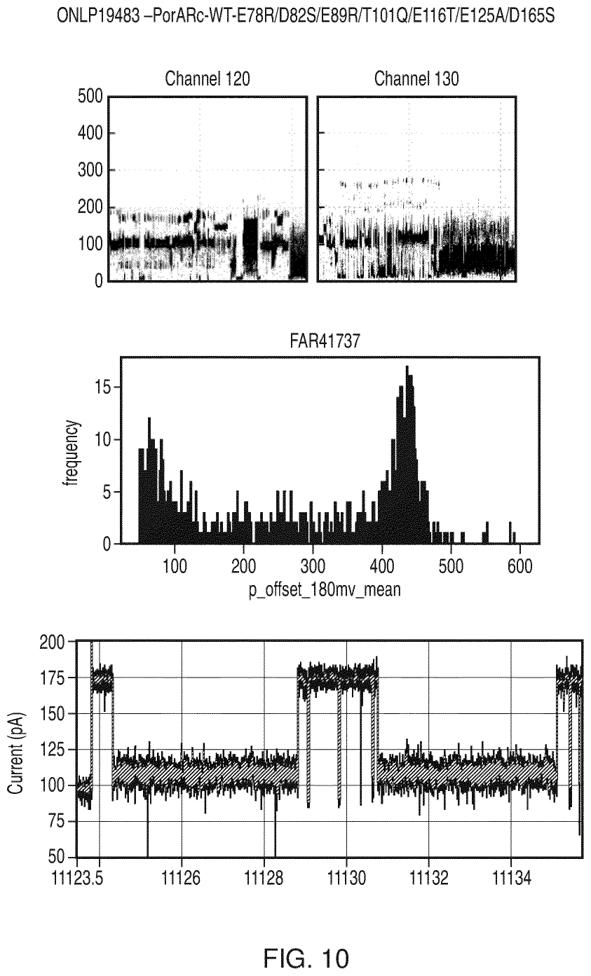

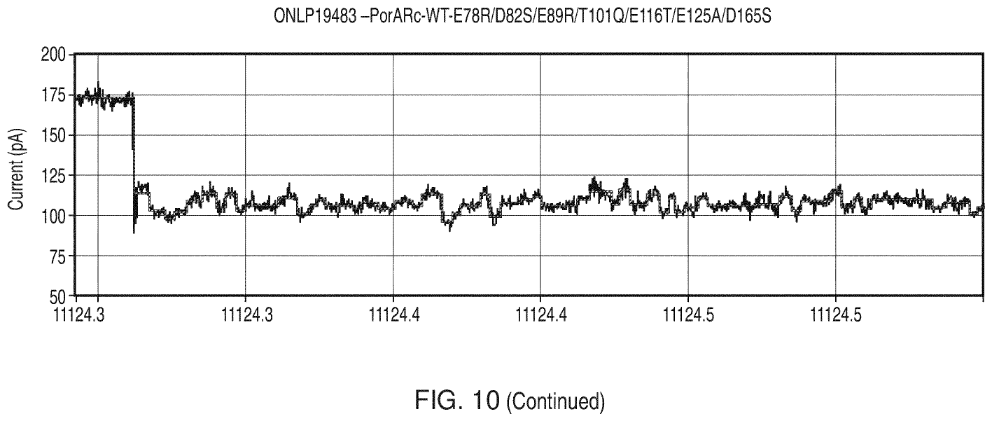

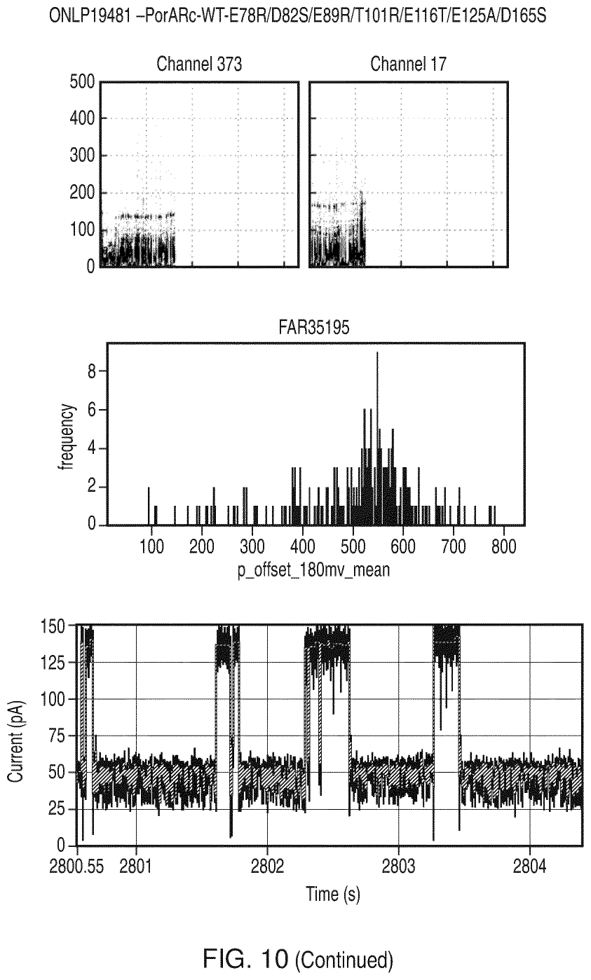

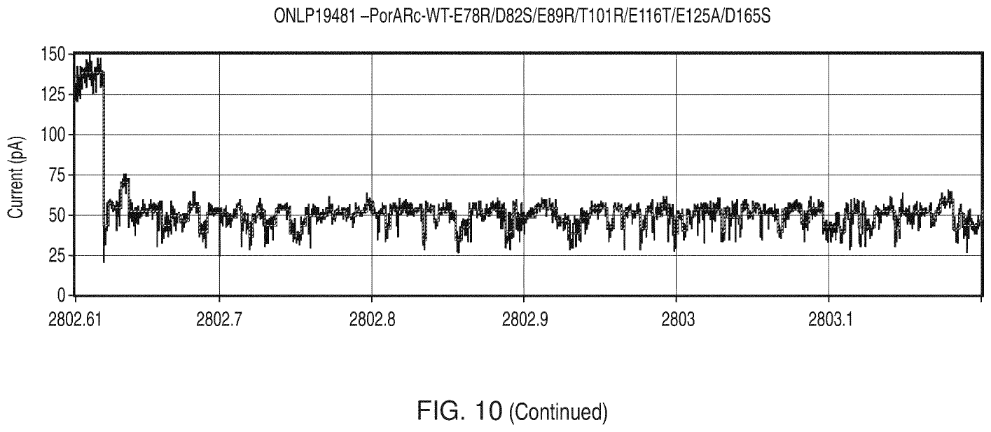

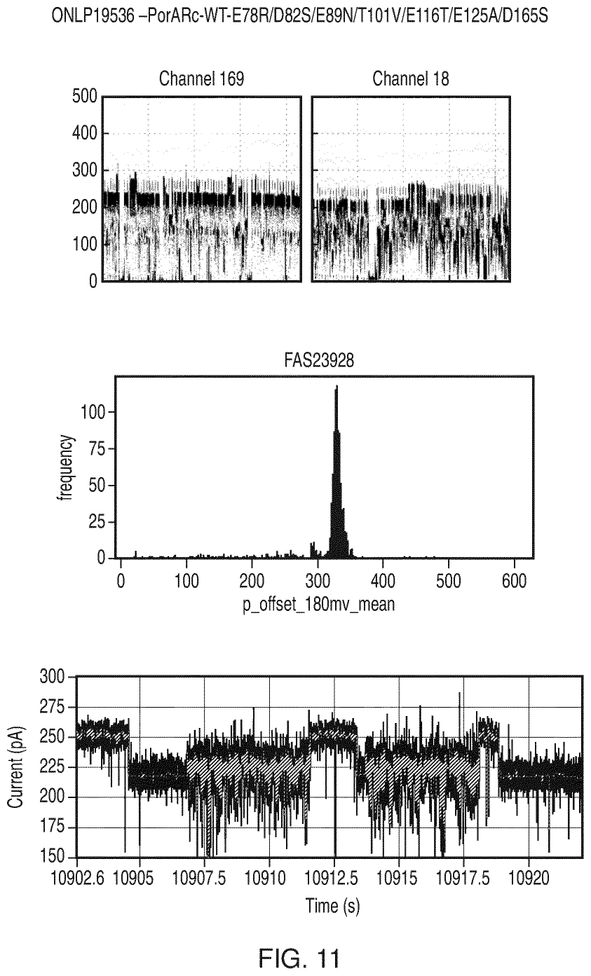

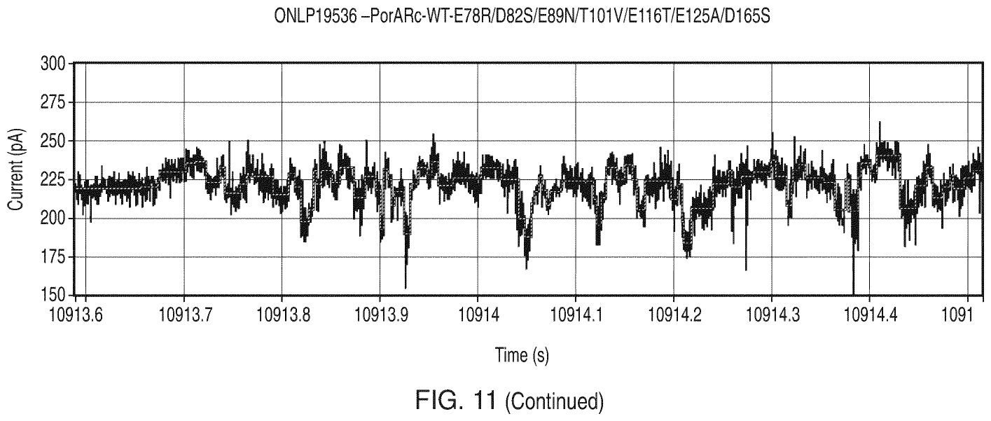

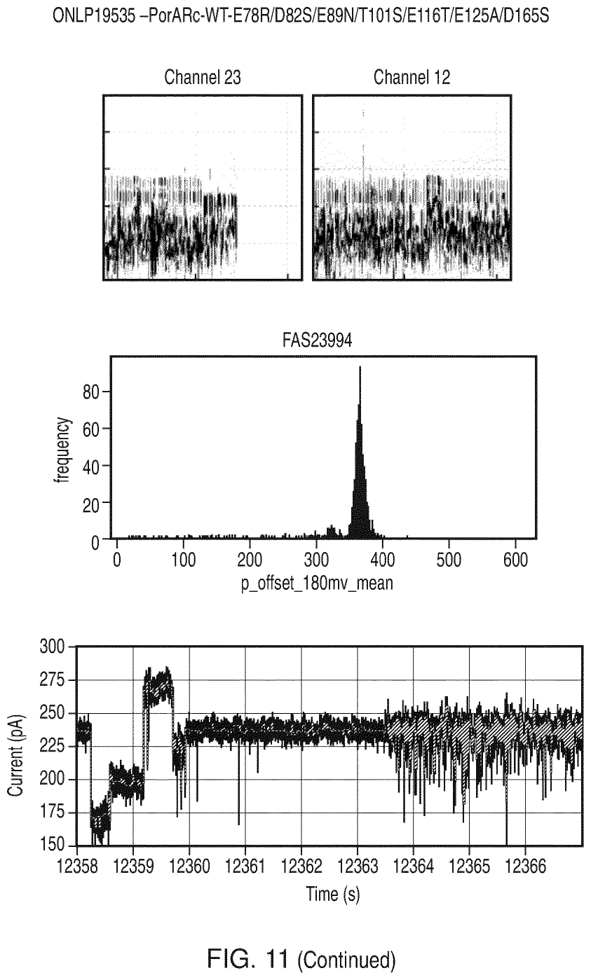

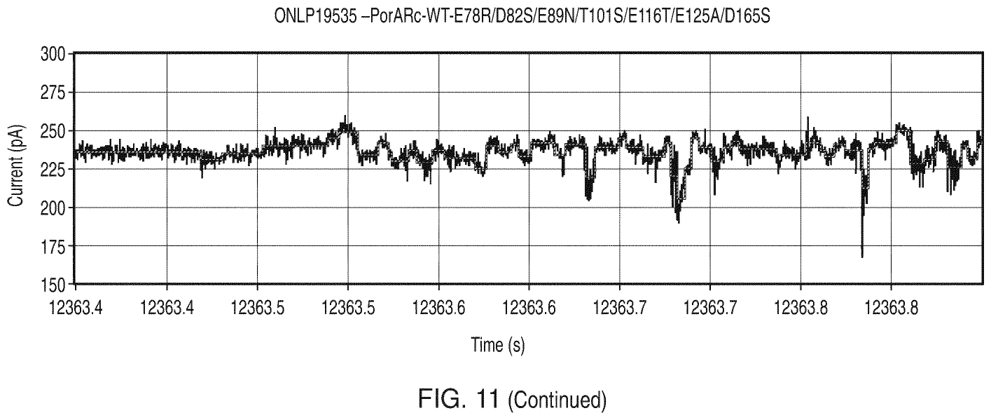

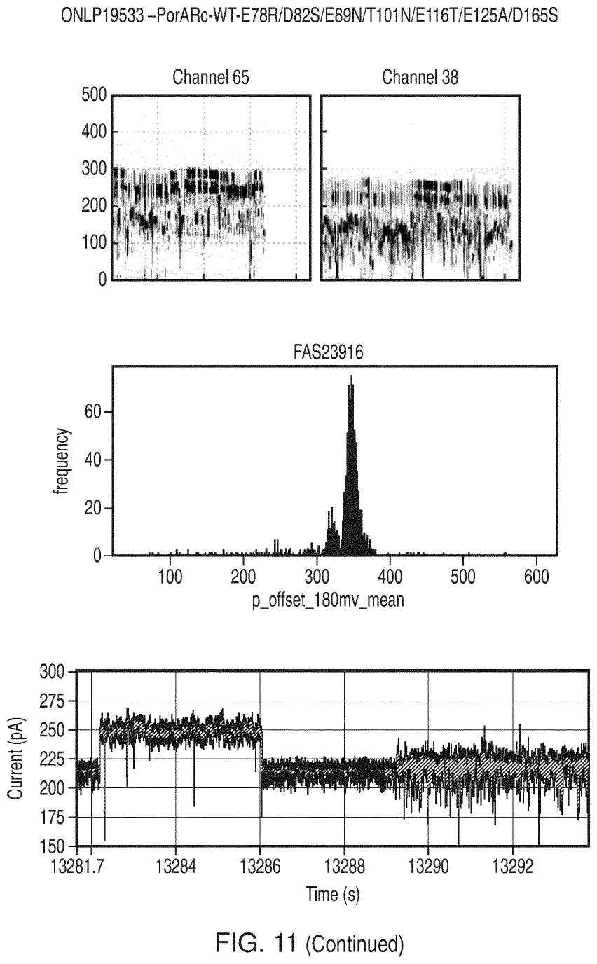

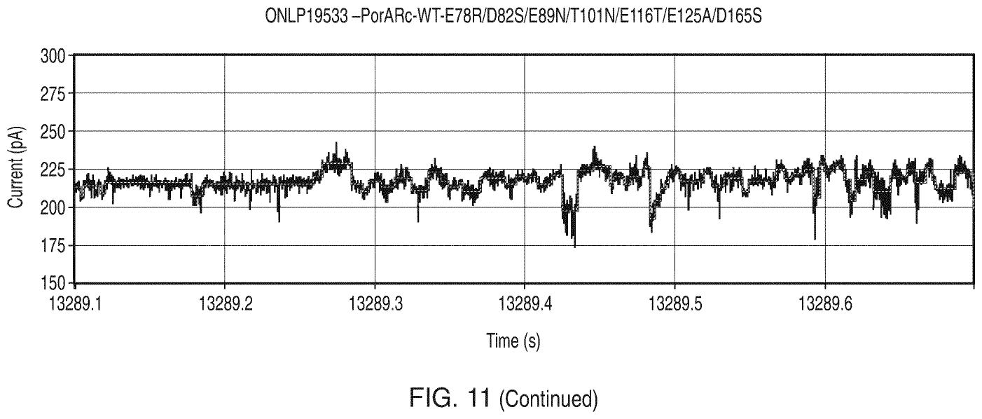

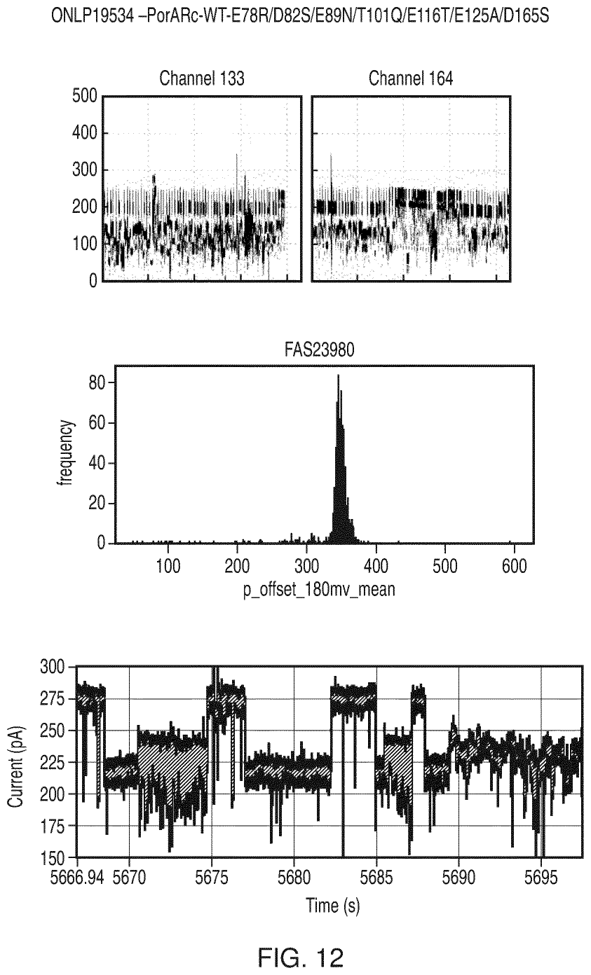

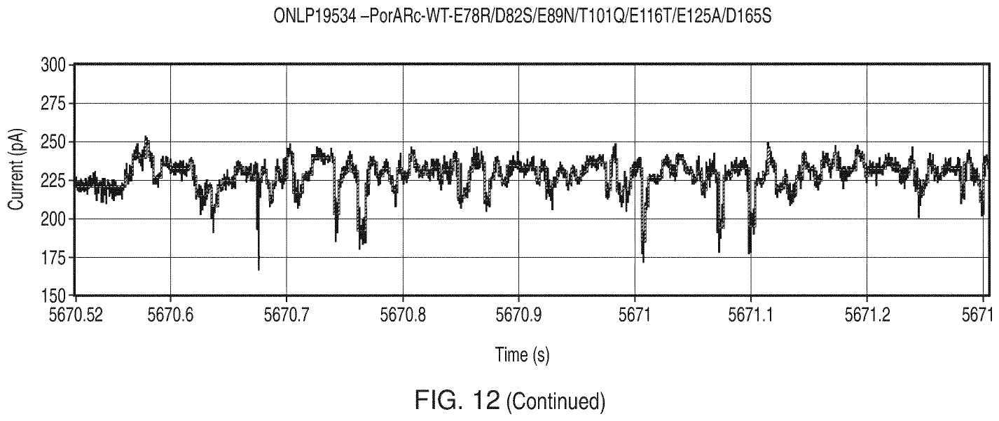

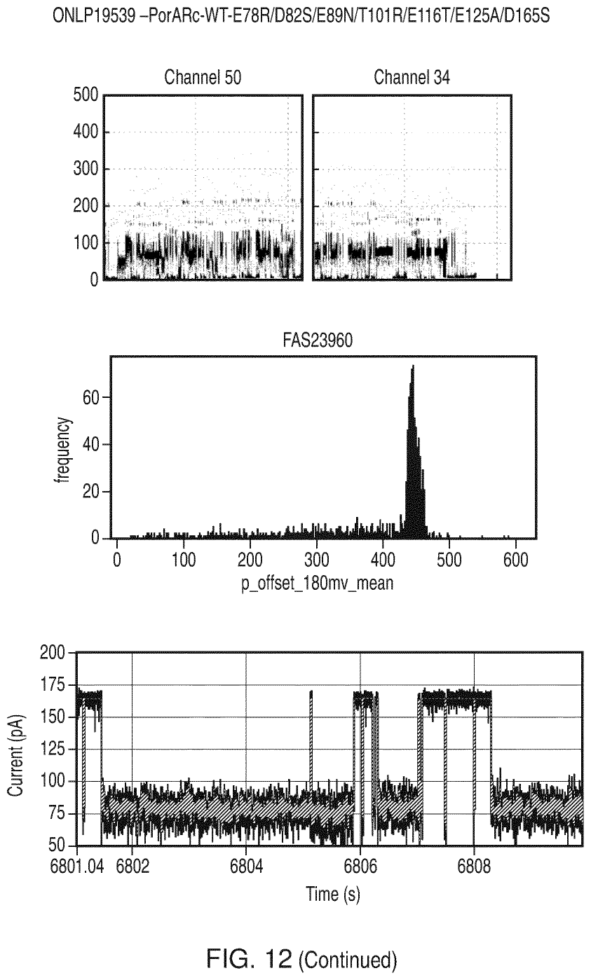

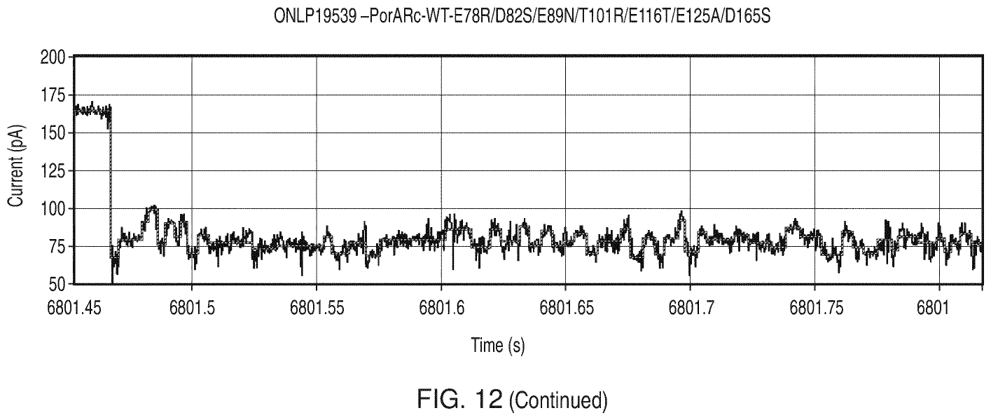

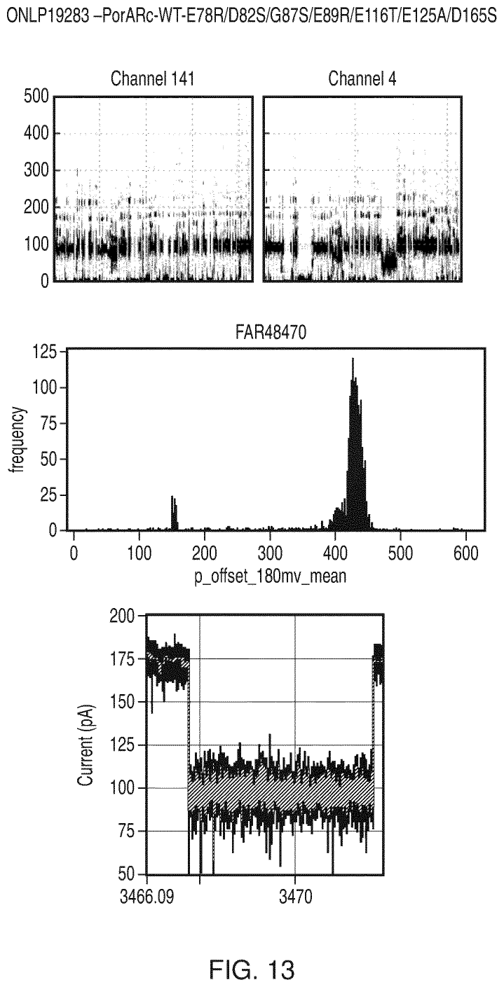

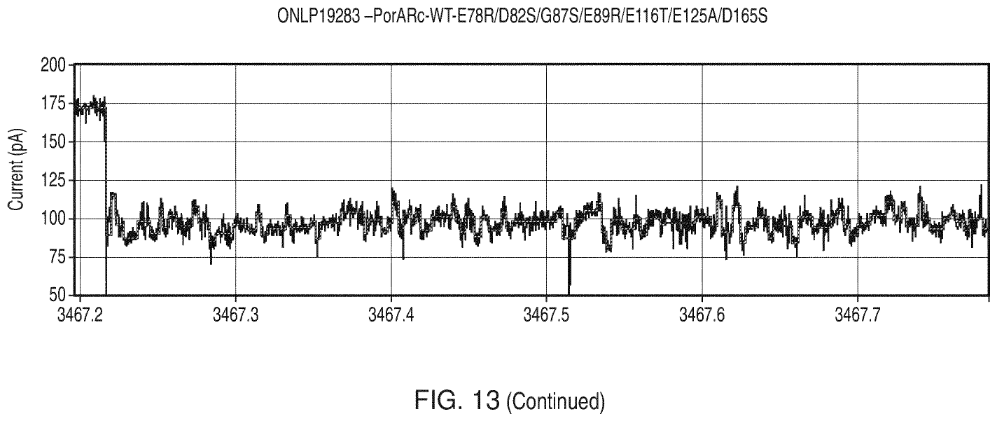

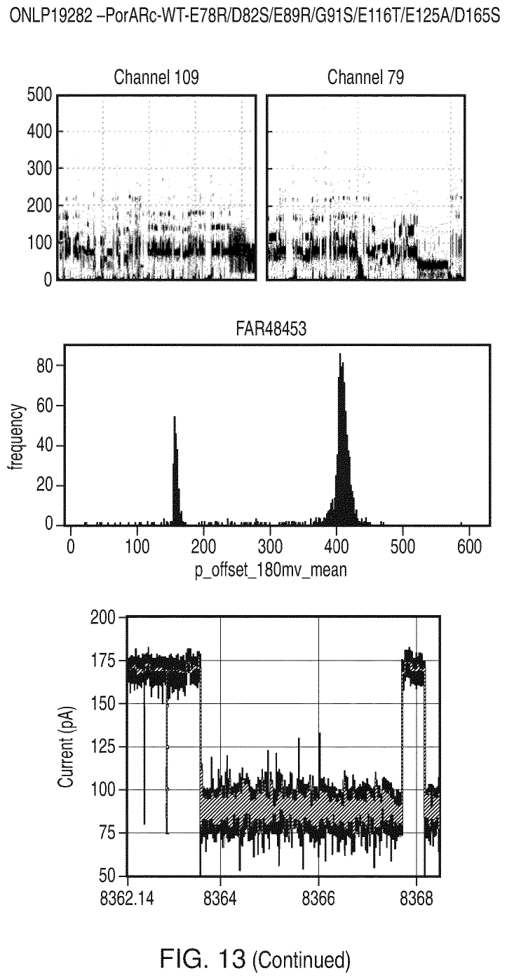

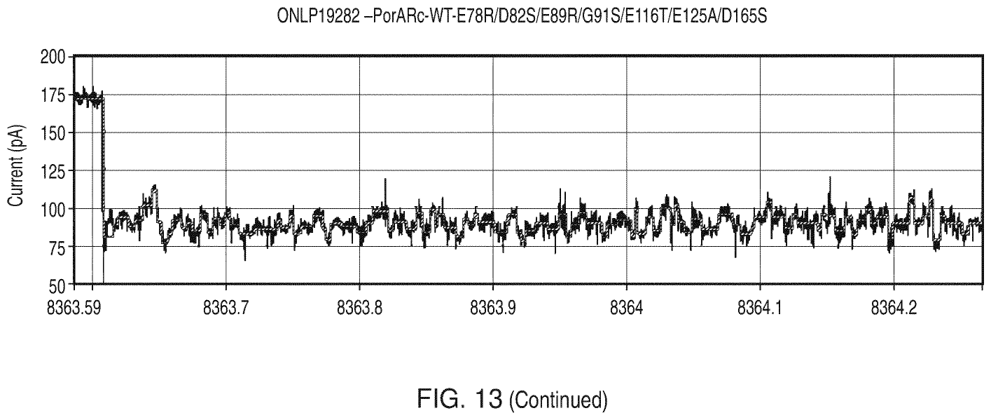

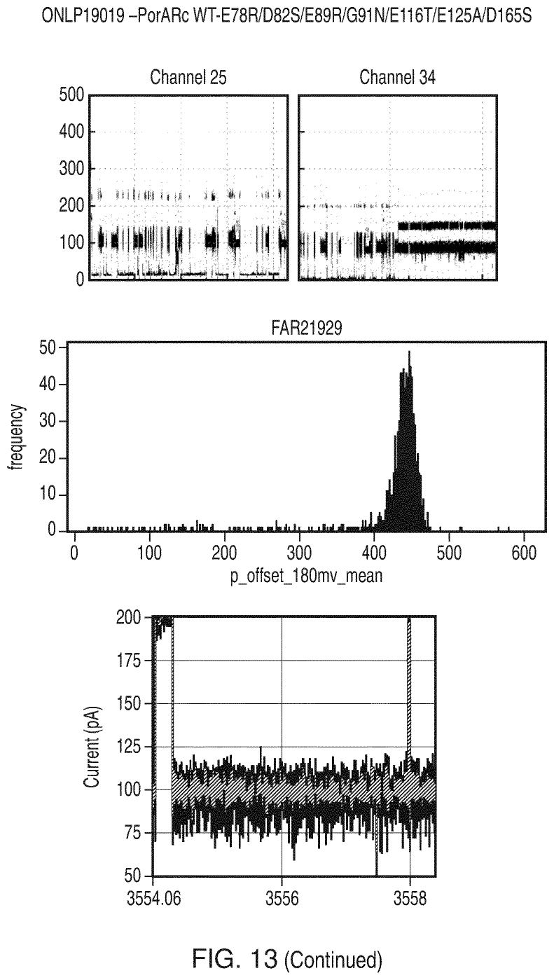

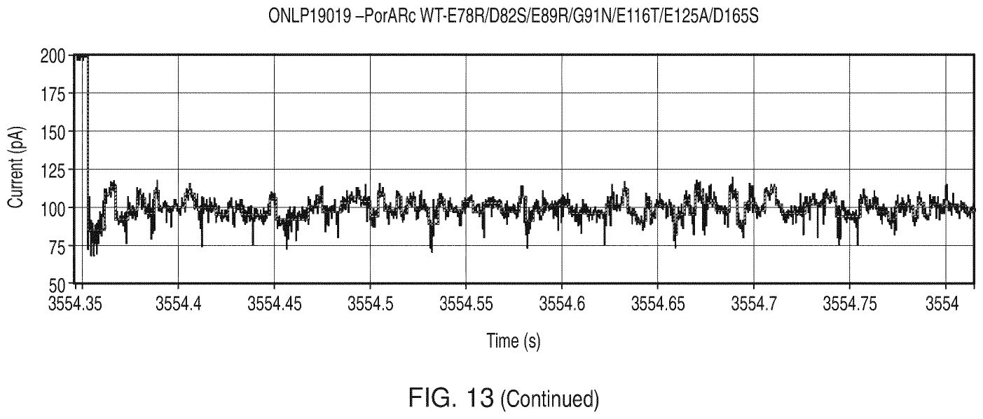

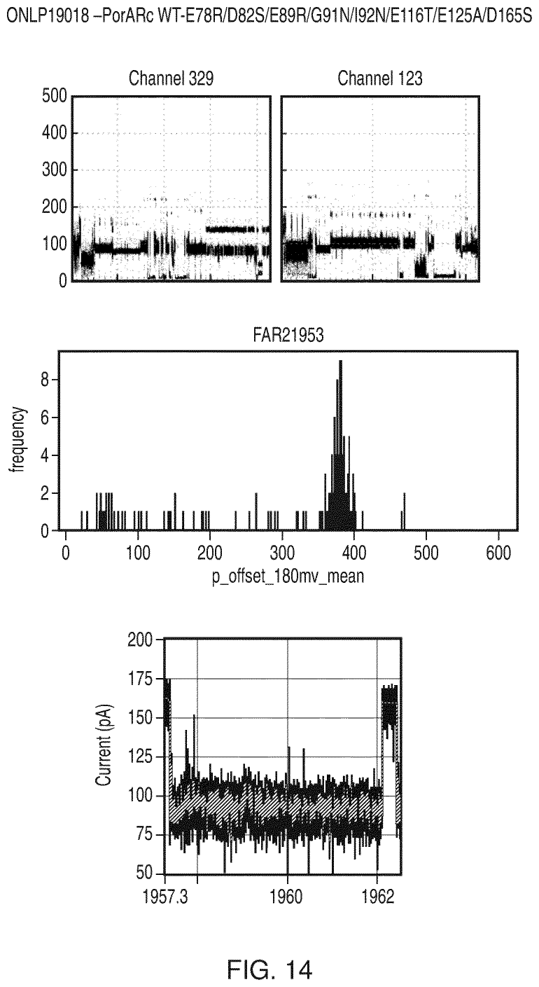

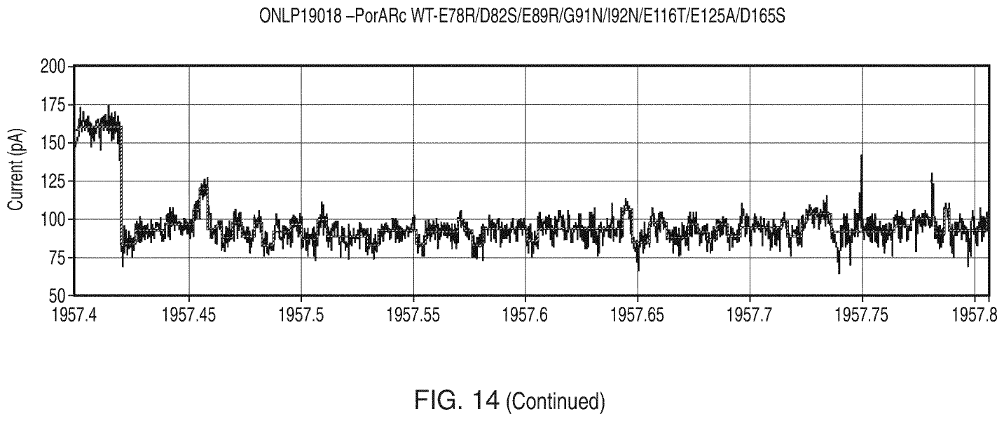

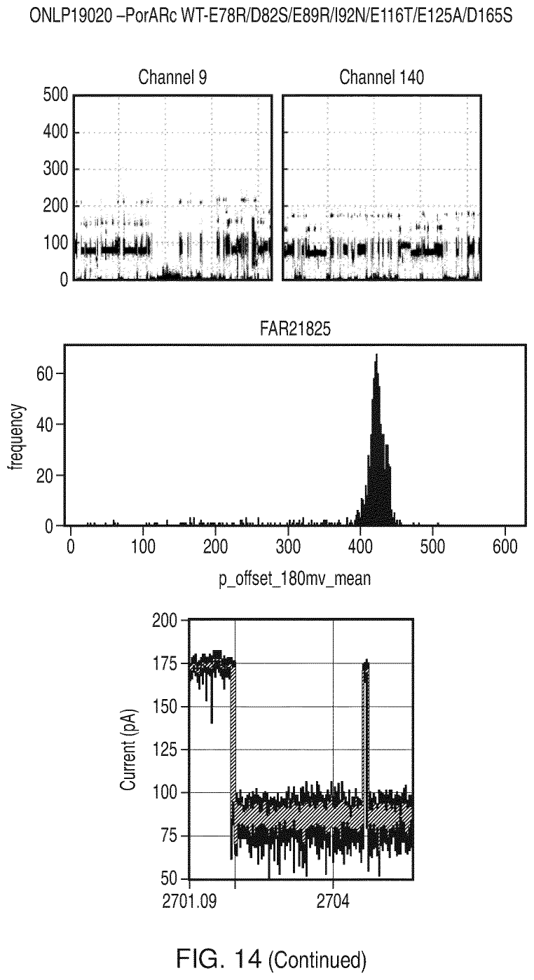

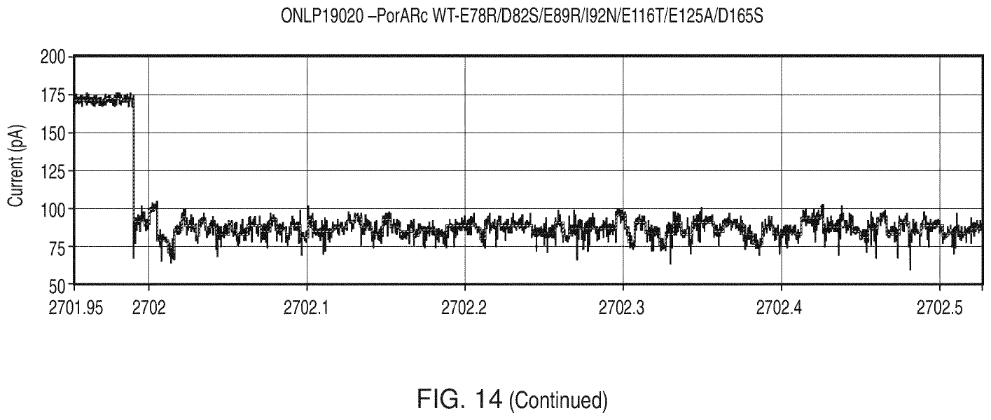

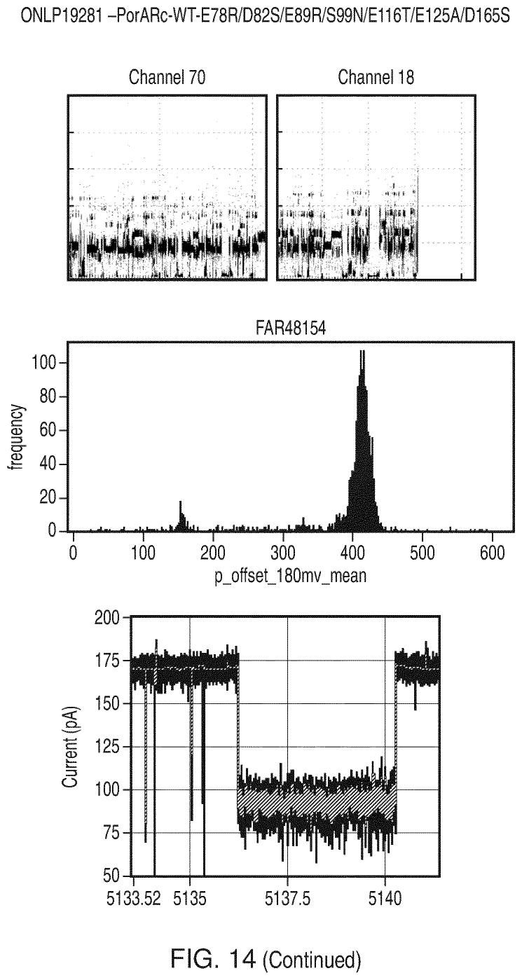

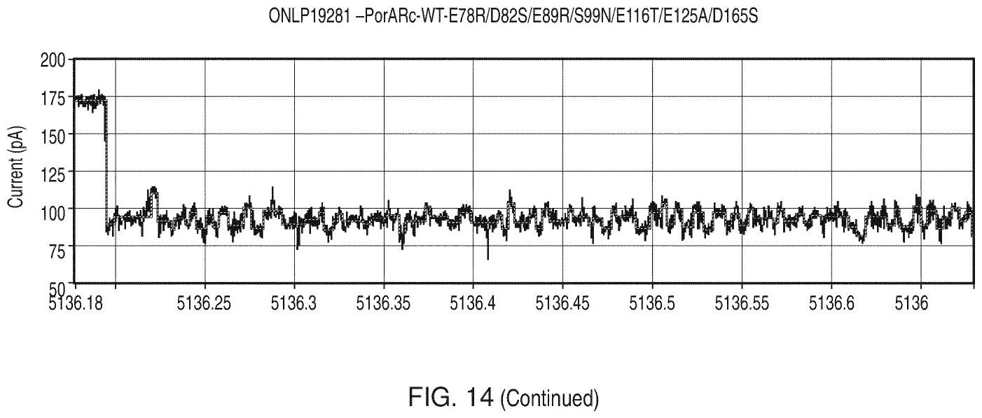

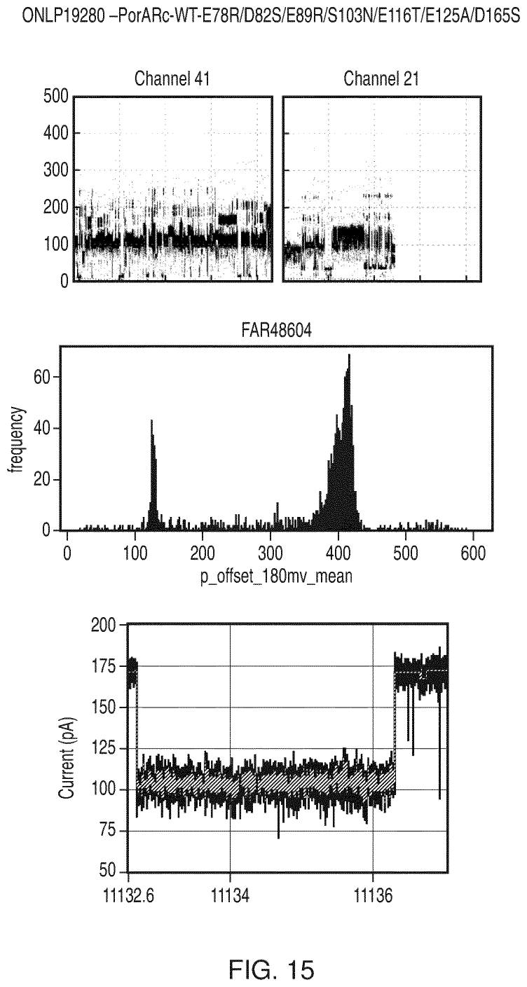

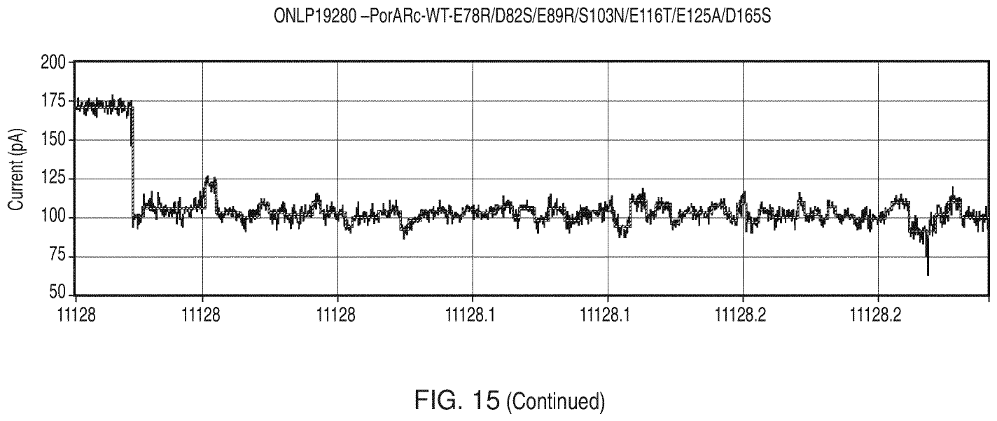

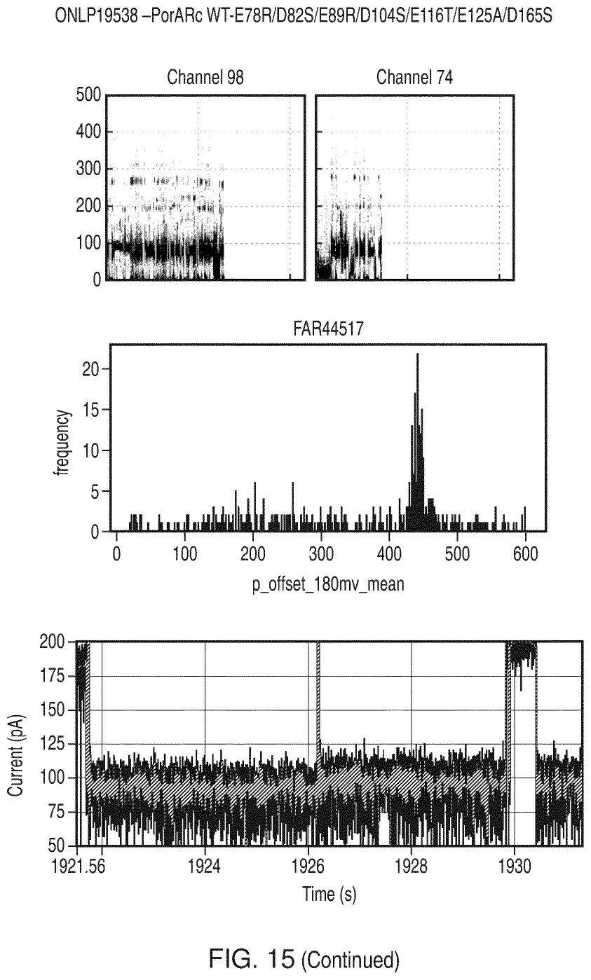

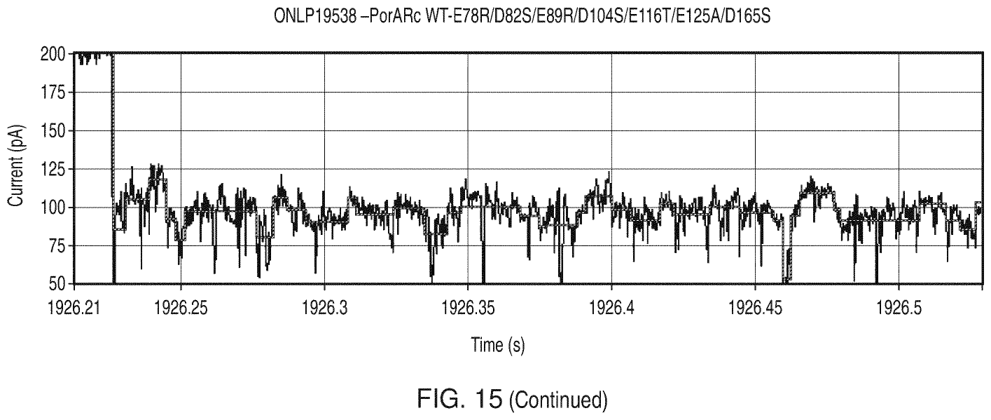

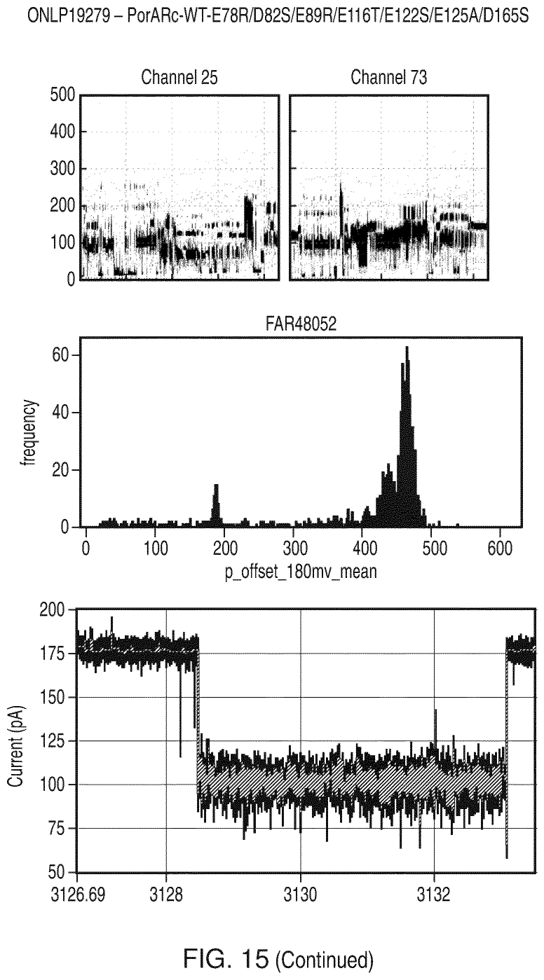

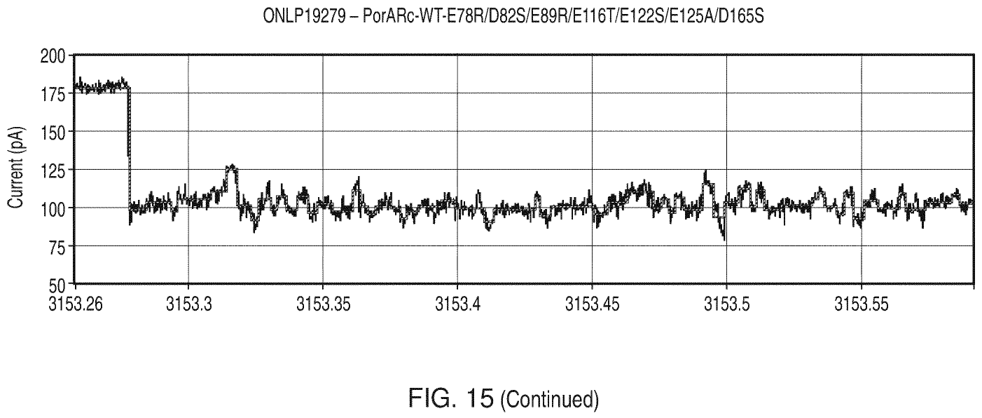

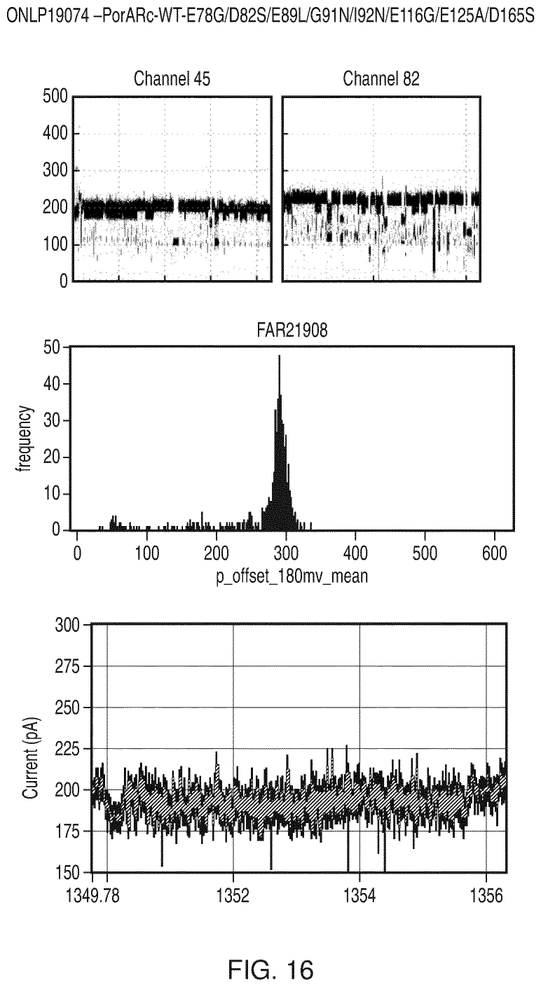

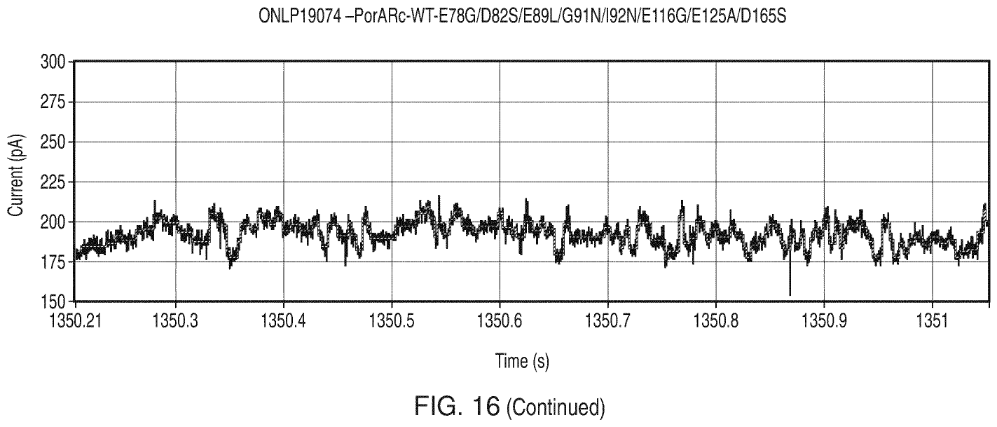

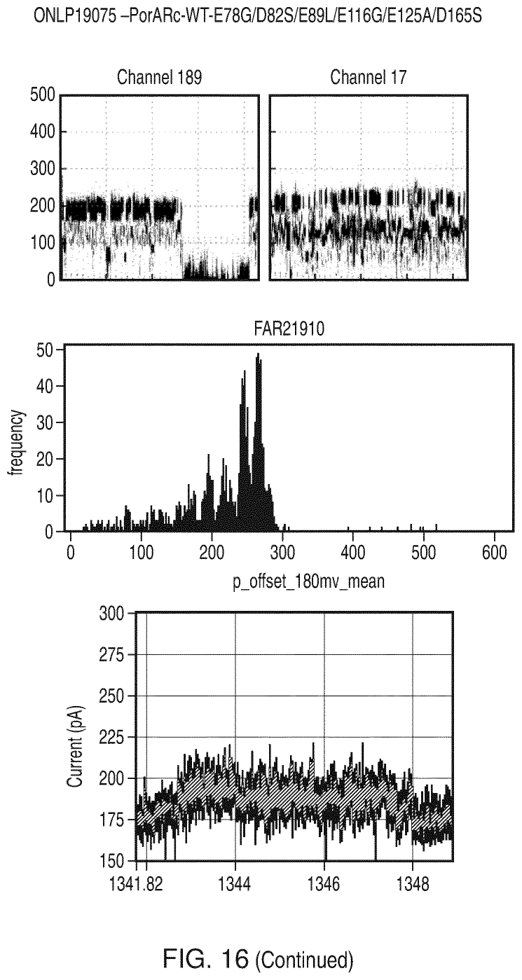

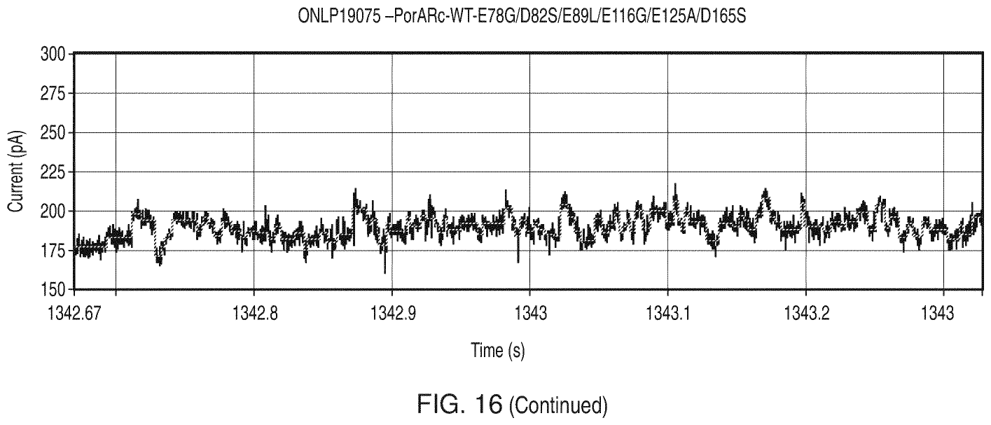

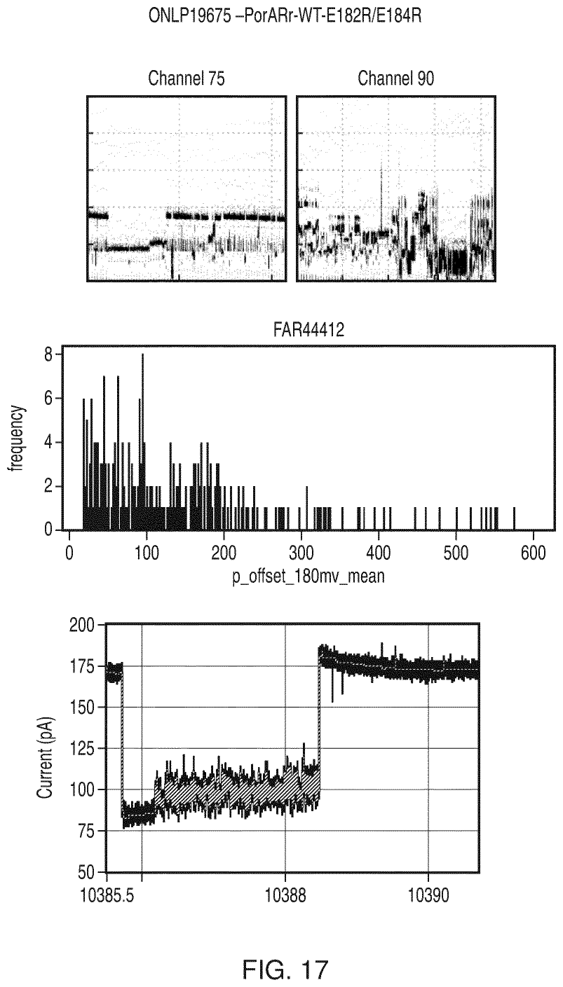

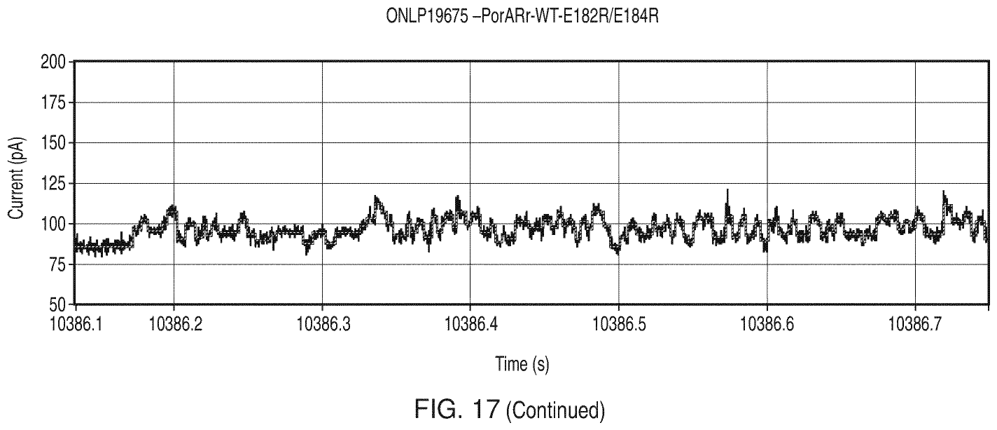

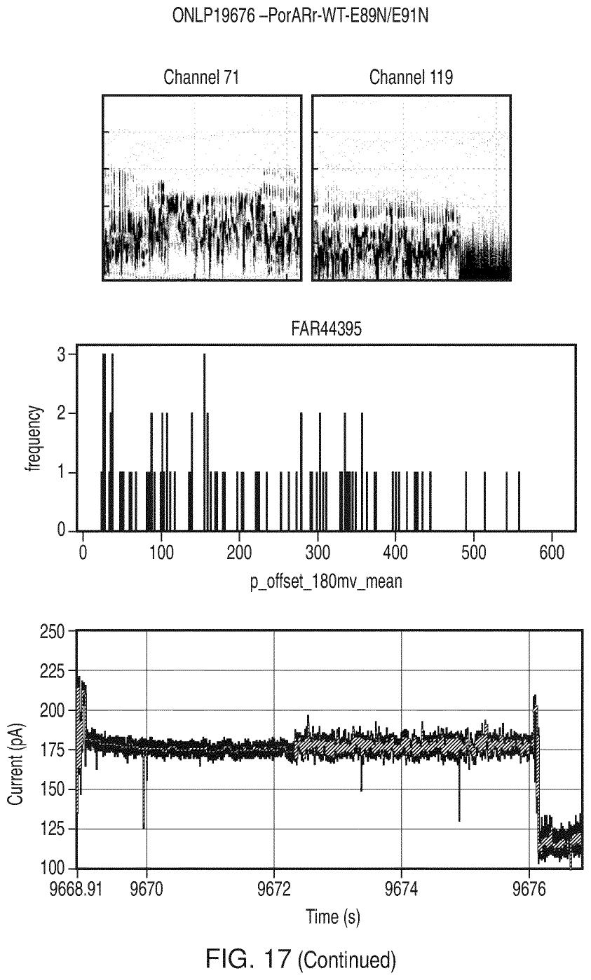

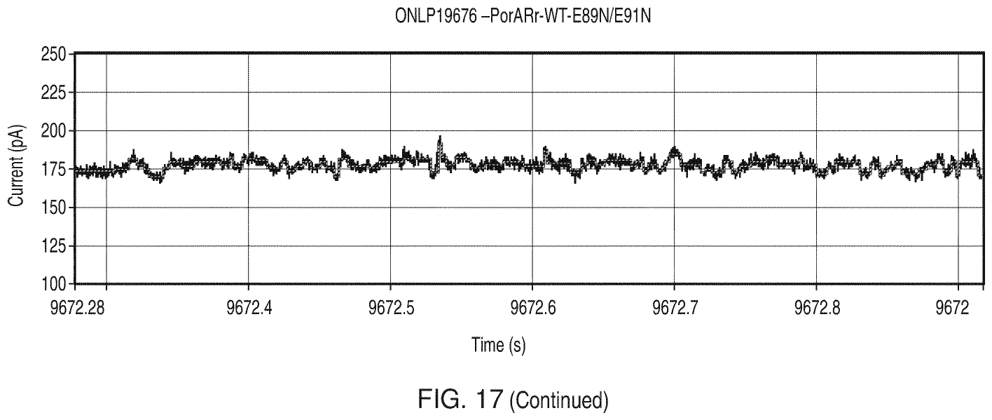

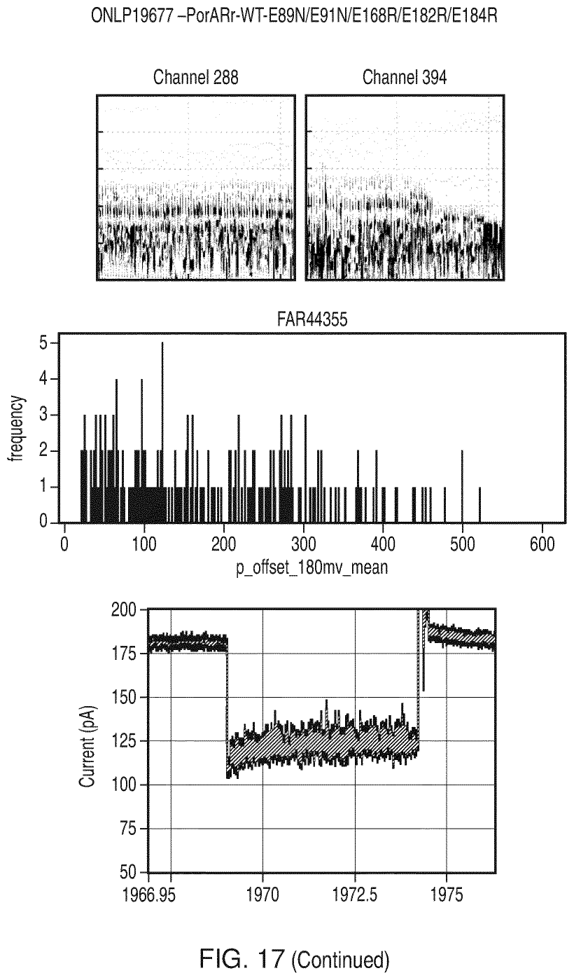

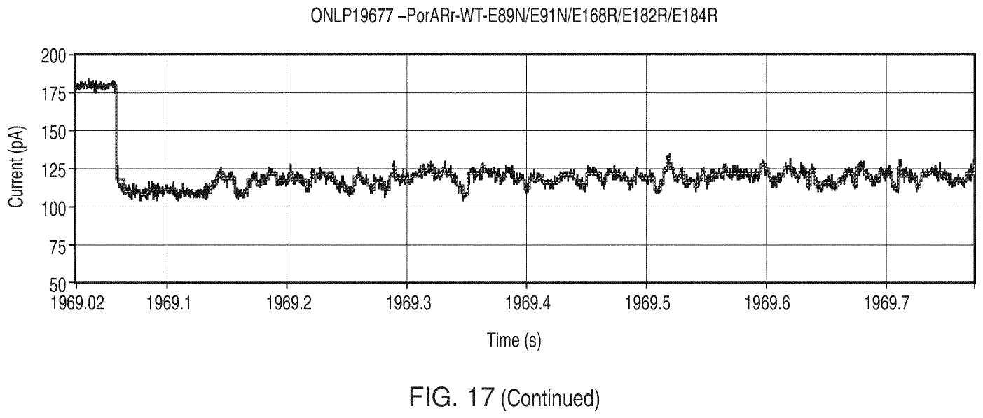

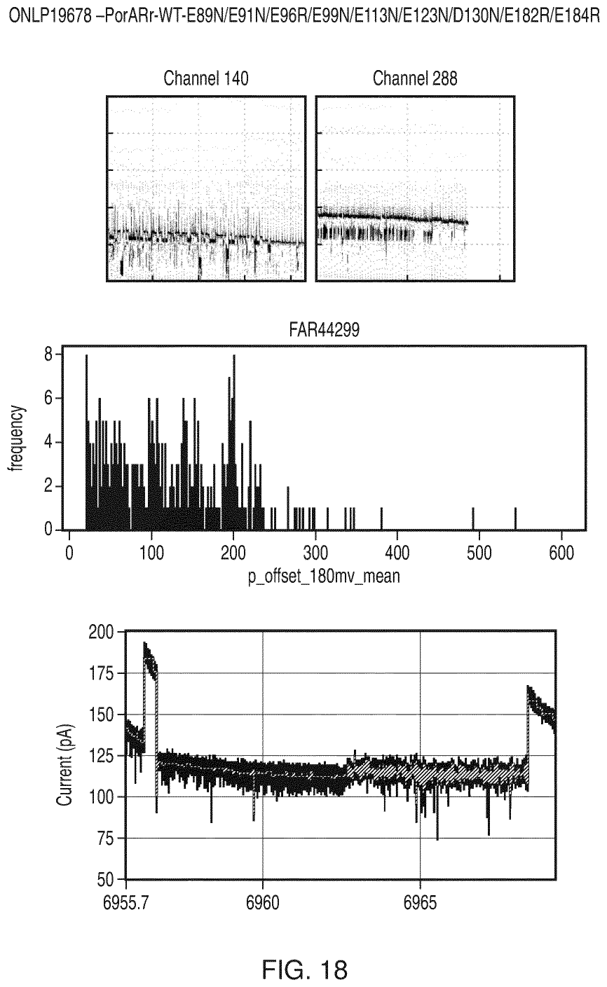

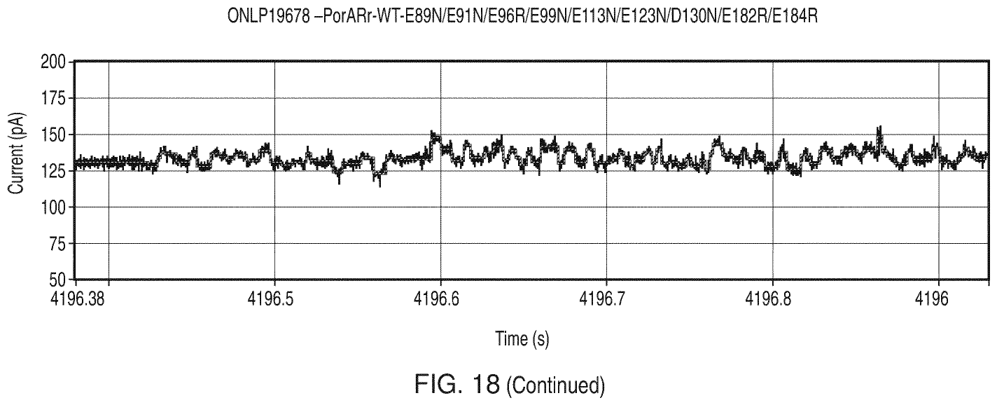

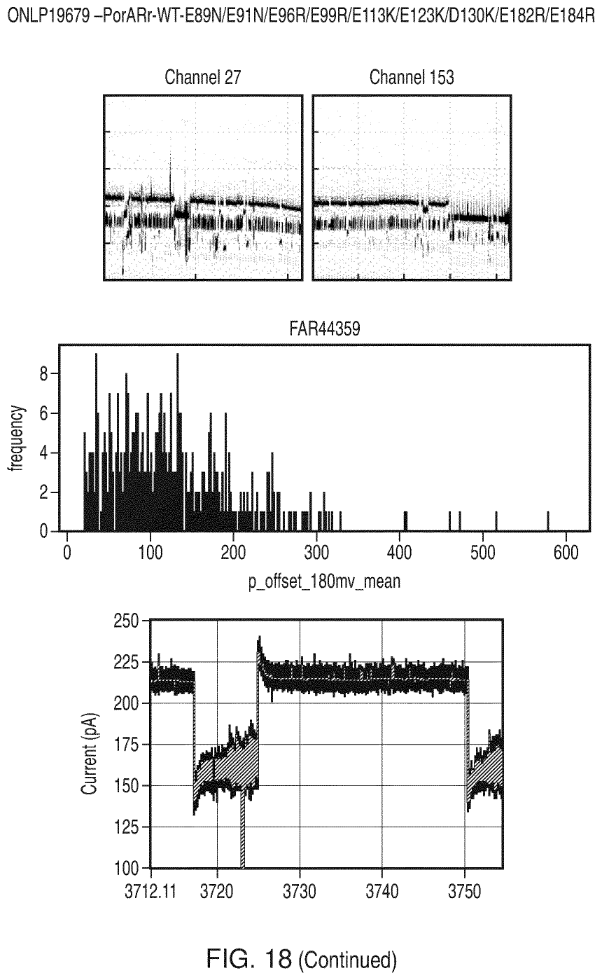

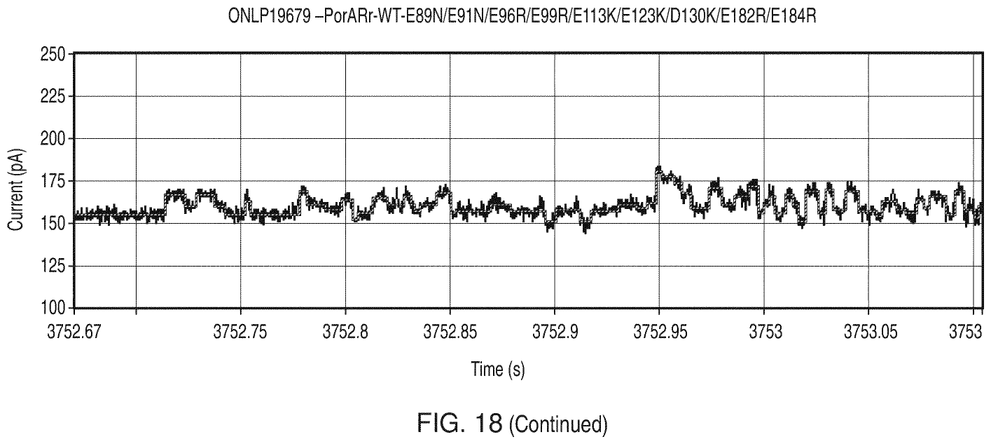

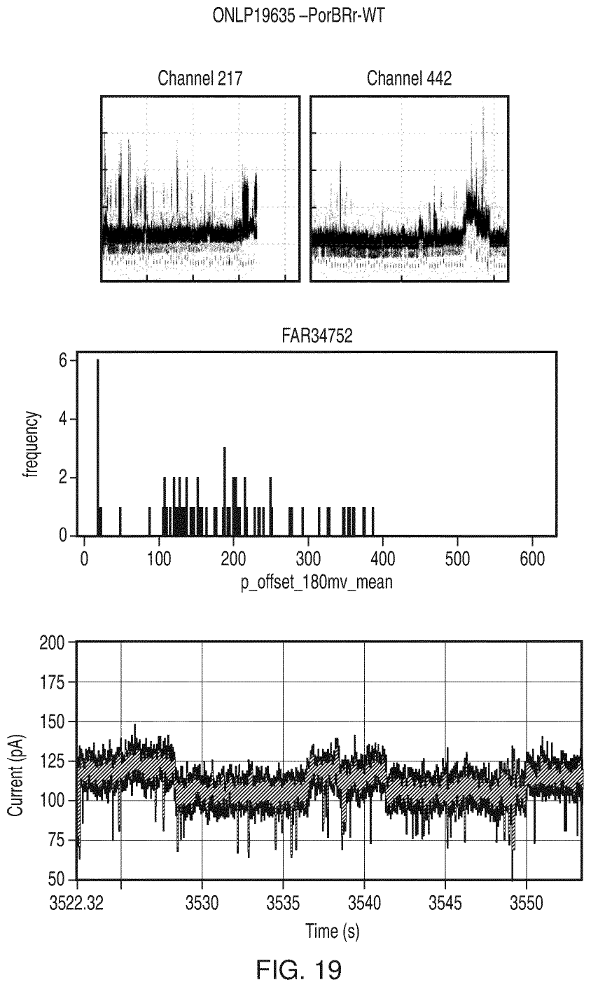

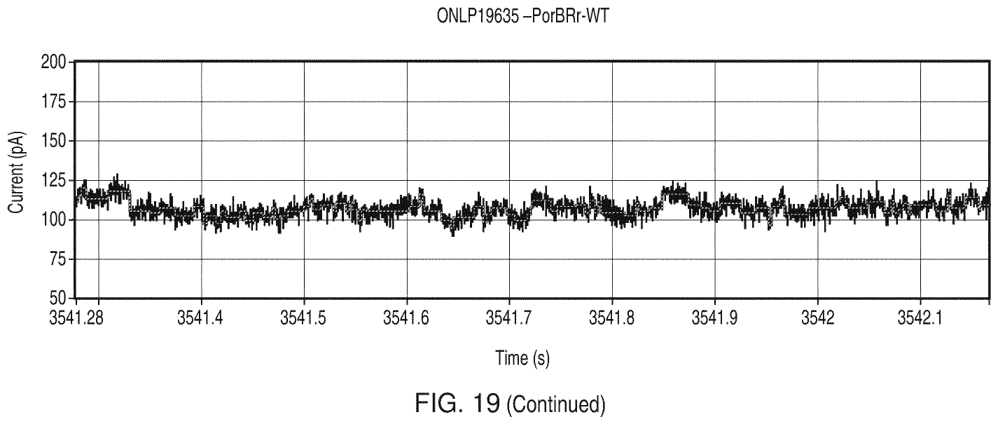

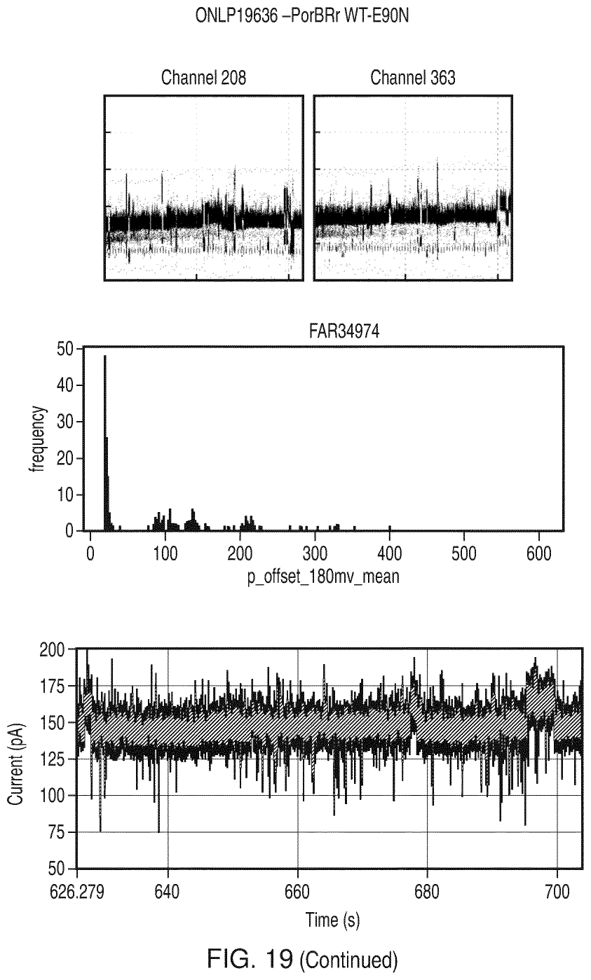

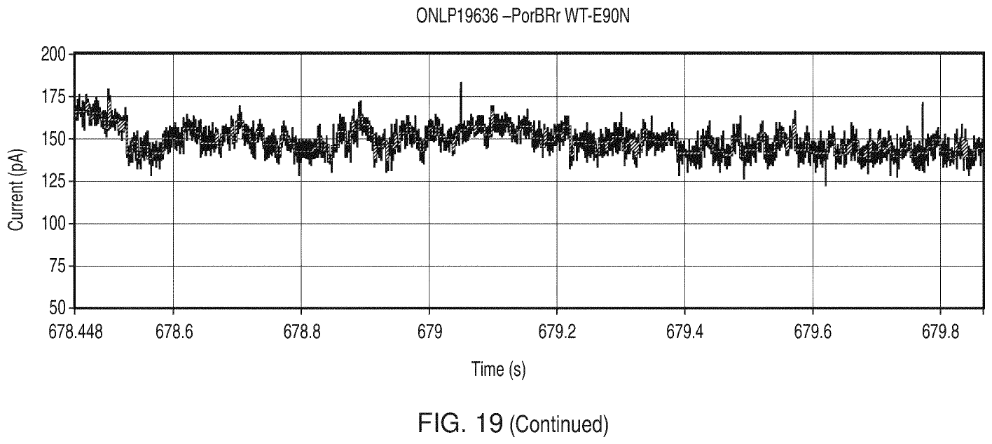

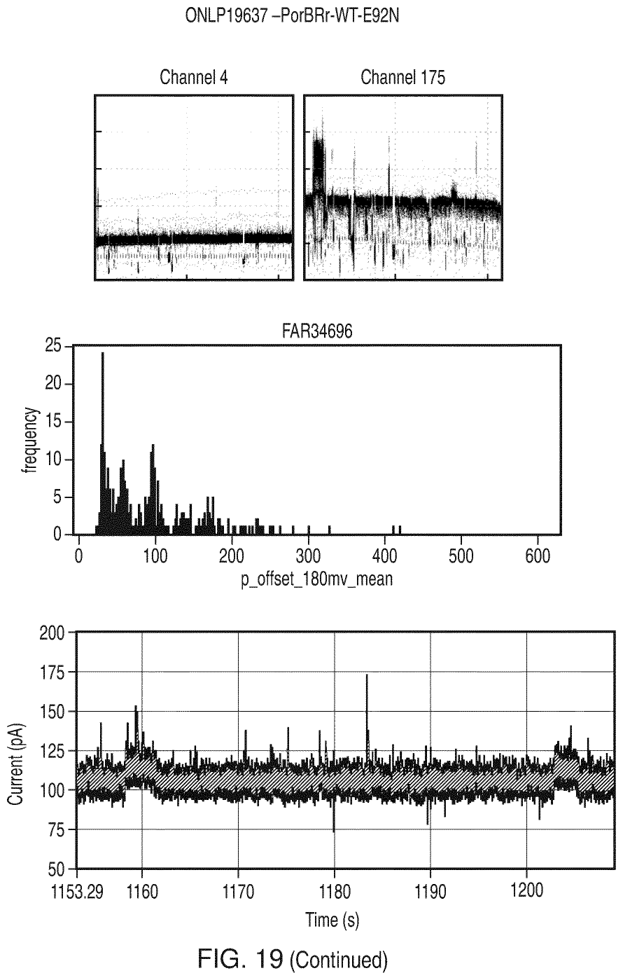

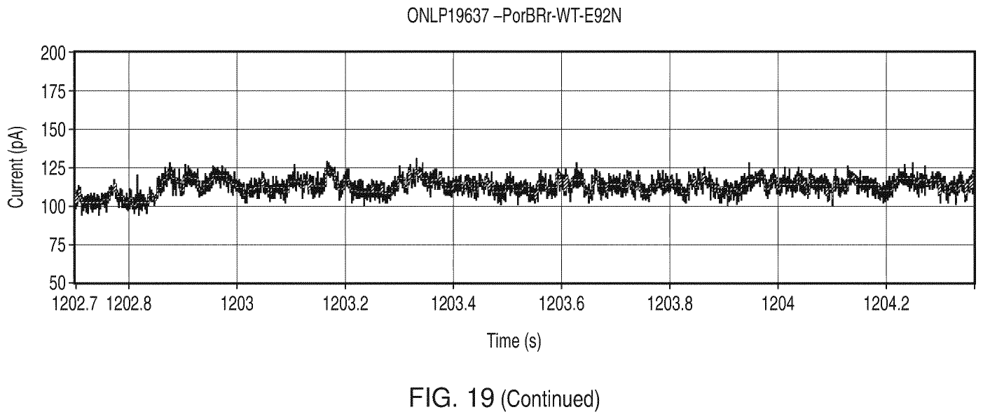

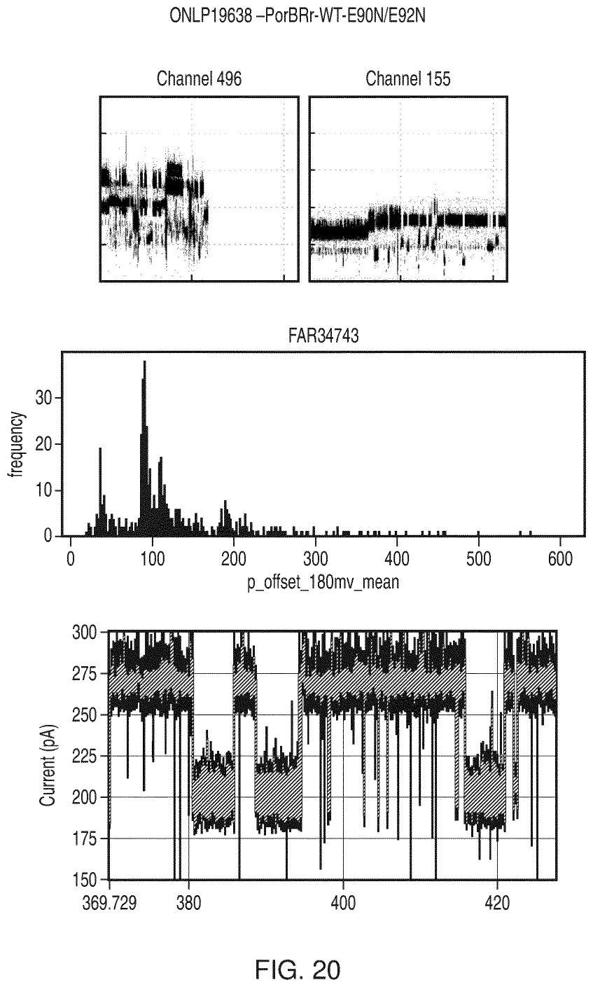

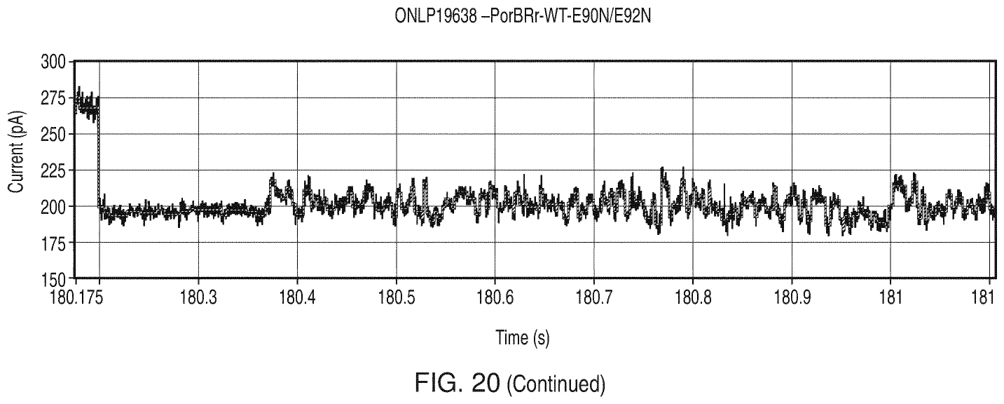

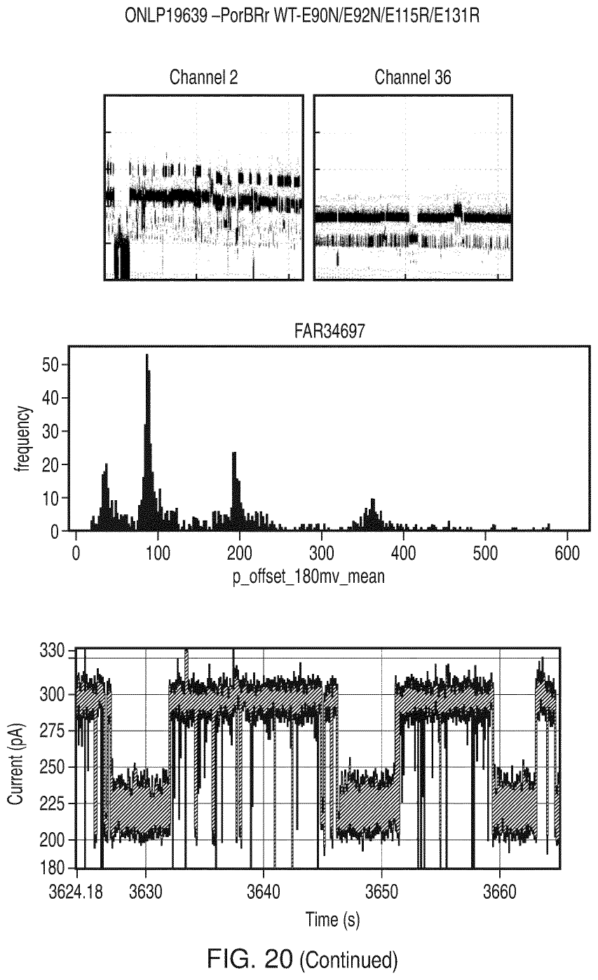

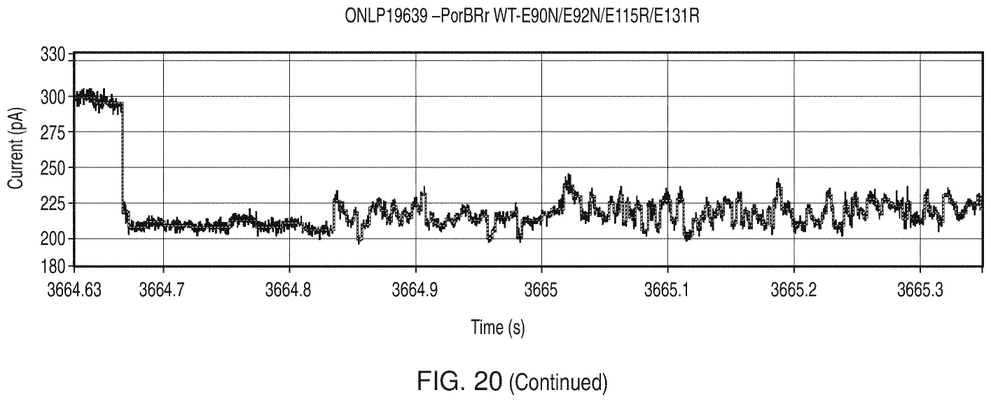

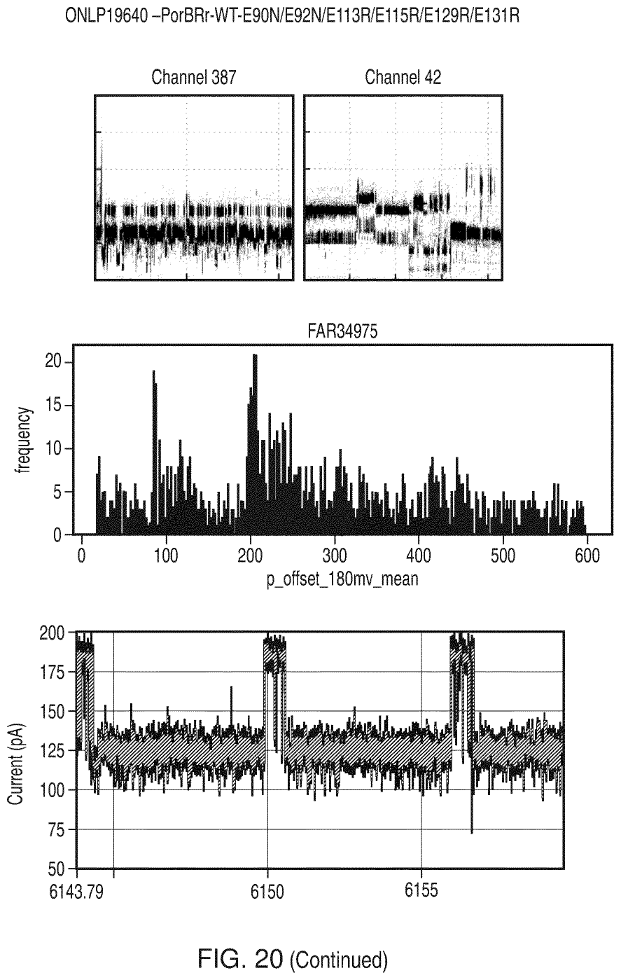

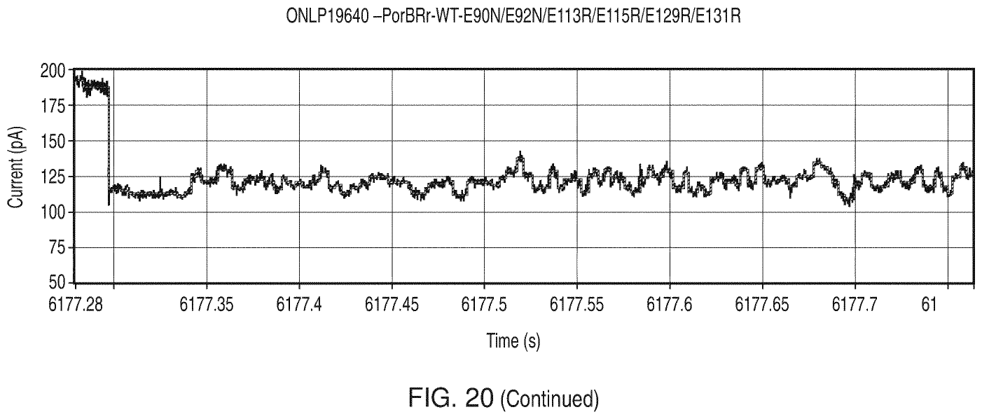

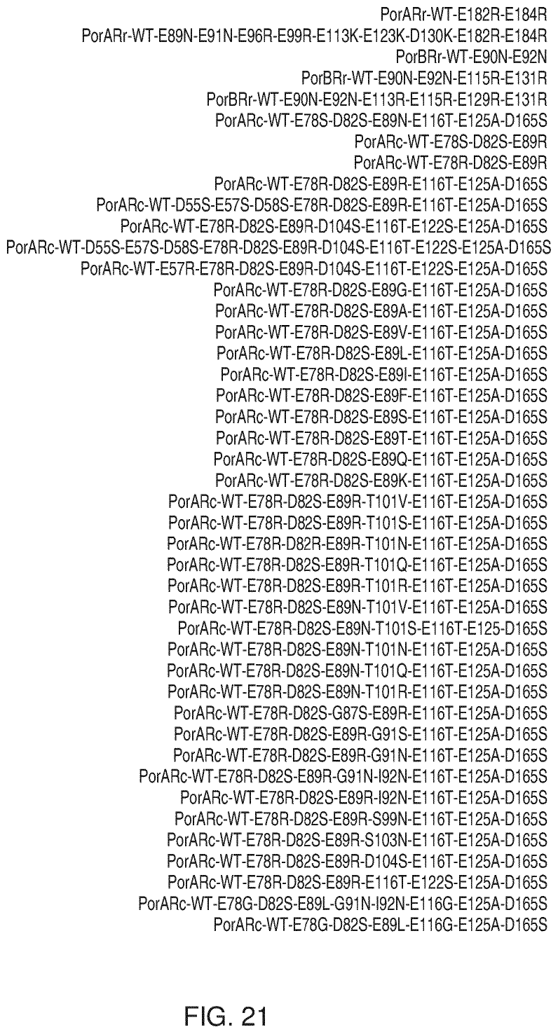

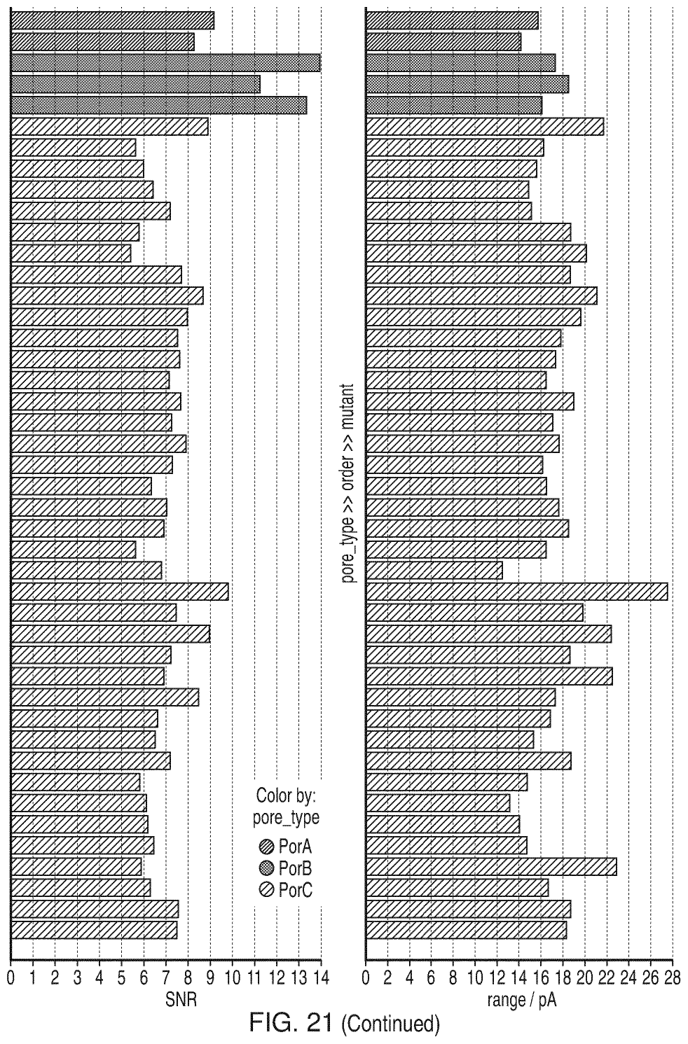

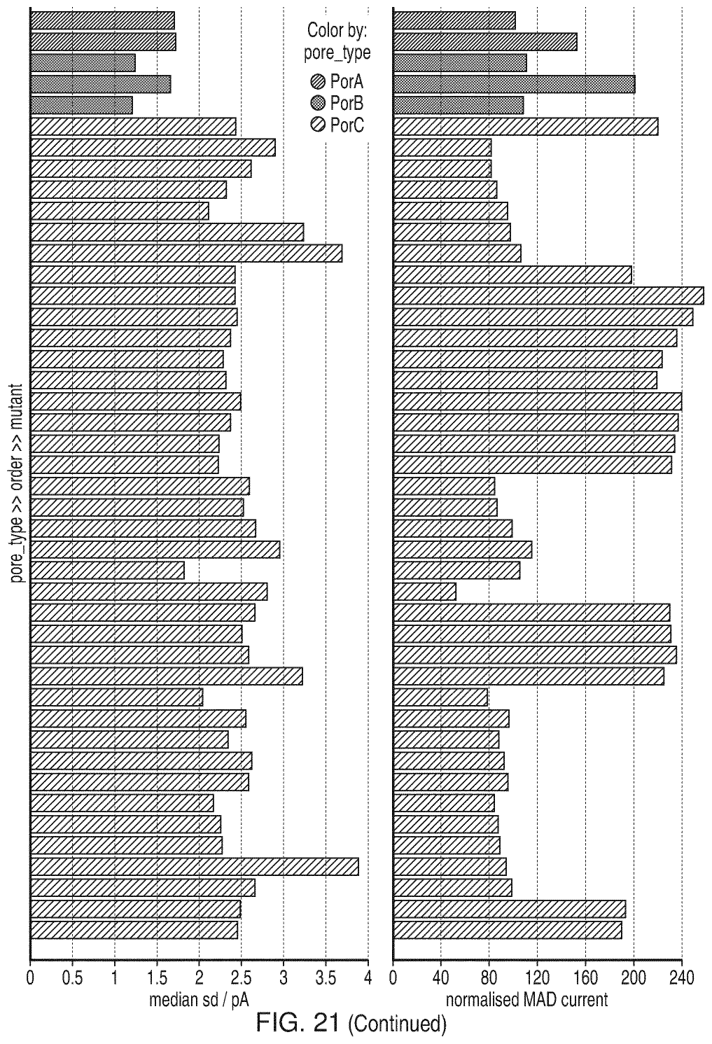

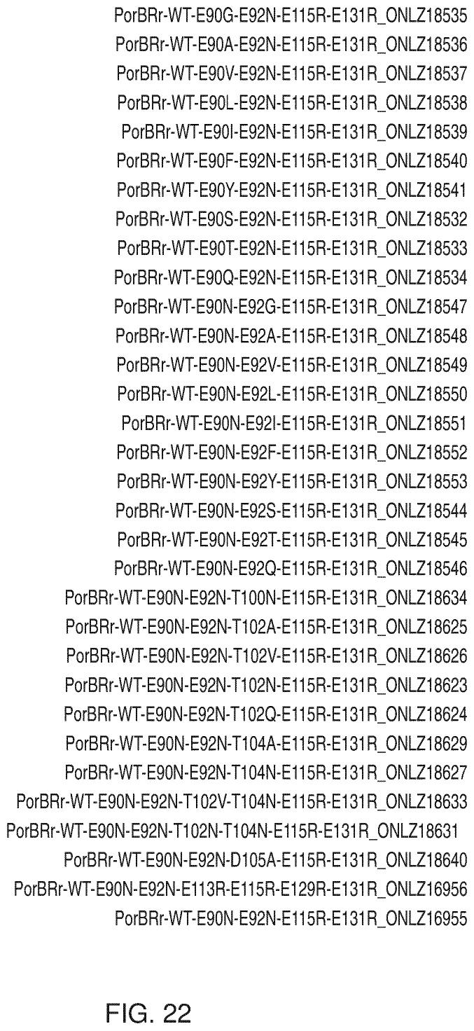

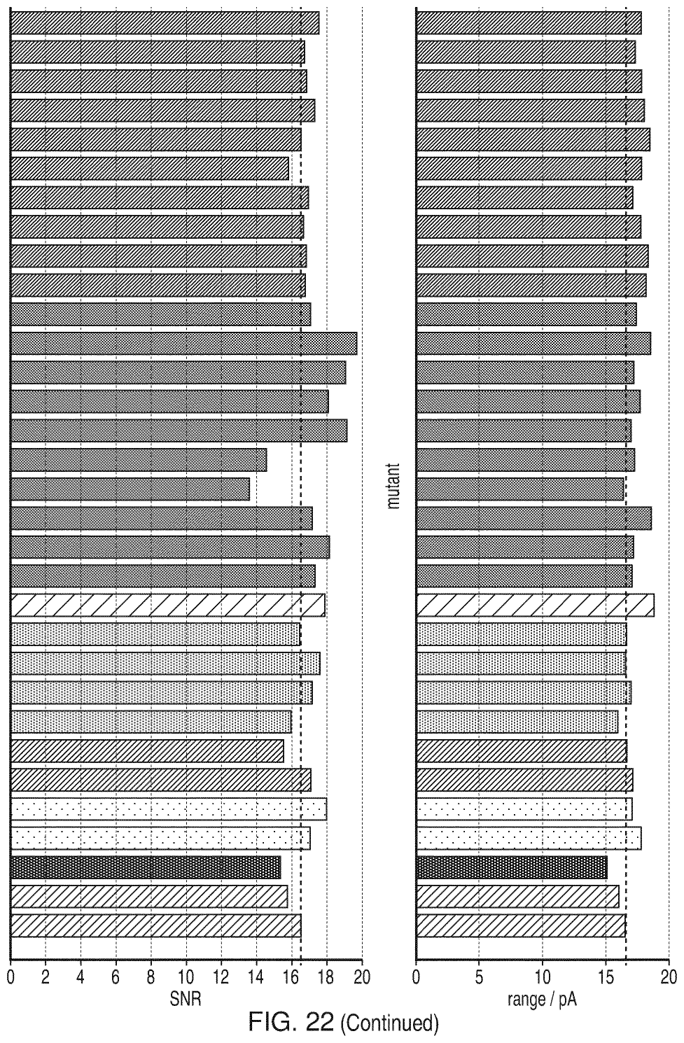

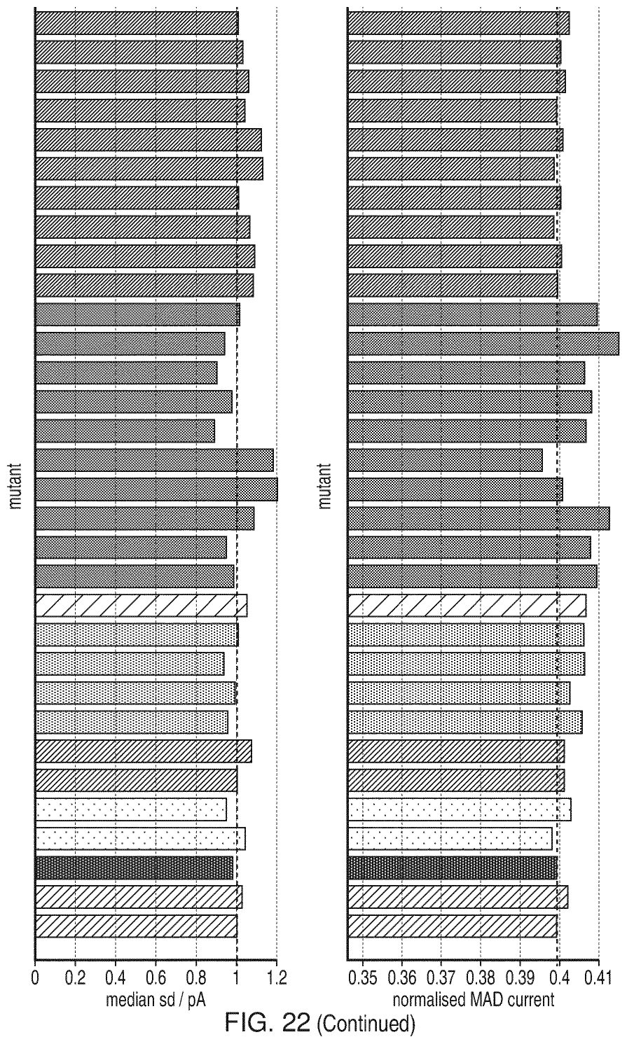

Each of FIGS. 1-20 show:
a) Snapshots of the ionic current versus time as DNA translocates through the pore, where the x-axis spans the entire experiment run time (typically 6 hours).
b) Histograms of the current flow from each channel in the flow cell upon application of an applied potential of −180 mV prior to addition of the analyte.
c) Zoomed-in snapshots of the ionic current versus time as DNA translocates through the pore. Note that the y-axis range spans 150 pA for ease of comparison between the pore mutants and the x-axis is typically more than 2 seconds.
d) Zoomed-in snapshots of the ionic current versus time as DNA translocates through the pore. Note that the y-axis range spans 150 pA for ease of comparison between the pore mutants and the x-axis is typically less than 2 seconds.

FIG. 21 shows strand metrics from PorARr, PorBRr and PorARc wild-type pores and their mutants as DNA translocates through the pore. SNR=current range divided by the noise on the current events, range=current range, median sd=noise on the current events and median current=median current as DNA translocates through the pore.

FIG. 22 shows strand metrics from PorBRr mutants as DNA translocates through the pore. SNR=current range divided by the noise on the current events, range=current range, median sd=noise on the current events and normalised MAD current=median absolute deviation of the current levels within the signal as DNA translocates through the pore and is normalised by dividing by the current range of the strand as DNA translocates through the pore.

### SEQUENCES IN THE SEQUENCE LISTING

SEQ ID NO: 1 shows the polynucleotide sequence encoding PorARc.

SEQ ID NO: 2 shows the amino acid sequence of PorARc.

SEQ ID NO: 3 shows the polynucleotide sequence encoding PorARr.

SEQ ID NO: 4 shows the amino acid sequence of PorARr.

SEQ ID NO: 5 shows the polynucleotide sequence encoding PorBRr.

SEQ ID NO: 6 shows the amino acid sequence of PorBRr.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e., E78S means that E at position 78 is replaced with S.

In the paragraphs herein where different amino acids at a specific position are separated by the / symbol, the / symbol means “or”. For instance, E89A/V means E89A or E89V.

In the paragraphs herein where different positions or substitutions are separated by the / symbol, the / symbol means, “and” such that E89A/E116T is E89A and E 116T.

All amino-acid substitutions, deletions and/or additions disclosed herein are with reference to a mutant monomer comprising a variant of the sequence shown in SEQ ID NO: 2, 4 or 6, unless stated to the contrary.

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### Definitions

Where an indefinite or definite article is used when referring to a singular noun e.g., “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Polynucleotide”, “nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

“Gene” as used here includes both the promoter region of the gene as well as the coding sequence. It refers both to the genomic sequence (including possible introns) as well as to the cDNA derived from the spliced messenger, operably linked to a promoter sequence.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference. The terms “polypeptide”, and “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers. Polypeptides can also undergo maturation or post-translational modification processes that may include, but are not limited to glycosylation, proteolytic cleavage, lipidization, signal peptide cleavage, propeptide cleavage, phosphorylation, and such like. By “recombinant polypeptide” is meant a polypeptide made using recombinant techniques, e.g., through the expression of a recombinant or synthetic polynucleotide. When the chimeric polypeptide or biologically active portion thereof is recombinantly produced, it is also preferably substantially free of culture medium, e.g., culture medium represents less than about 20%, more preferably less than about 10%, and most preferably less than about 5% of the volume of the protein preparation. By “isolated” is meant material that is substantially or essentially free from components that normally accompany it in its native state. For example, an “isolated polypeptide”, as used herein, refers to a polypeptide, which has been purified from the molecules which flank it in a naturally occurring state, e.g., a Rhodococcus porin monomer which has been removed from the molecules present in the production host that are adjacent to said polypeptide. An isolated peptide can be generated by amino acid chemical synthesis or can be generated by recombinant production. An isolated pore can be generated by in vitro reconstitution after purification of the components of the pore or can be generated by recombinant co-expression.

The term “protein” is used to describe a folded polypeptide having a secondary or tertiary structure. The protein may be composed of a single polypeptide or may comprise multiple polypeptides that are assembled to form a multimer. The multimer may be a homo-oligomer, or a hetero-oligomer. The protein may be a naturally occurring, or wild type protein, or a modified, or non-naturally, occurring protein. The protein may, for example, differ from a wild type protein by the addition, substitution, or deletion of one or more amino acids.

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation and are also derived from a common ancestral gene.

“Variant”, “Homologue” and “Homologues” of a protein encompass peptides, oligopeptides, polypeptides, proteins, and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “transmembrane protein pore” defines a pore comprising multiple pore monomers. Each monomer may be a wild-type monomer, or a variant of thereof. The variant monomer may also be referred to as a modified monomer or a mutant monomer. The modifications, or mutations, in the variant include but are not limited to any one or more of the modifications disclosed herein, or combinations of said modifications.

The term “Rhodococcus pore” defines a pore comprising multiple PorARr, PorBRr or PorARc monomers. Each PorARr, PorBRr or PorARc monomer may be a wild-type monomer (SEQ ID NO: 2, 4 or 6), wild-type homologues of PorARr, PorBRr or PorARc or a variant of any thereof (e.g., a variant of any one of SEQ ID NOs: 2, 4 or 6). The variant PorARr, PorBRr or PorARc monomer may also be referred to as a modified PorARr, PorBRr or PorARc monomer or a mutant PorARr, PorBRr or PorARc monomer. The modifications, or mutations, in the variant include but are not limited to any one or more of the modifications disclosed herein, or combinations of said modifications.

For all aspects and embodiments of the present invention, a homologue is referred to as a polypeptide that has at least 50%, 60%, 70%, 80%, 90%, 95% or 99% complete sequence identity to PorARr, PorBRr or PorARc, such as the sequence shown in SEQ ID NO: 2, 4 or 6.

Likewise, a homologous polynucleotide can comprise a polynucleotide that has at least 50%, 60%, 70%, 80%, 90%, 95% or 99% complete sequence identity to the nucleic acid sequence encoding a wild-type protein. For example, a PorARr, PorBRr or PorARc homologous polynucleotide can comprise a polynucleotide that has at least 50%, 60%, 70%, 80%, 90%, 95% or 99% complete sequence identity to PorARr, PorBRr or PorARc as shown in SEQ ID NO: 1, 3 or 5.

Sequence identity can also be to a fragment or portion of the full-length polynucleotide or polypeptide. Hence, a sequence may have only 50% overall sequence identity with a full-length reference sequence, but a sequence of a particular region, domain or subunit could share 80%, 90%, or as much as 99% sequence identity with the reference sequence. Homology to the nucleic acid sequence of SEQ ID NO: 1, 3 or 5 for PorARr, PorBRr or PorARc homologues, respectively, is not limited simply to sequence identity. Many nucleic acid sequences can demonstrate biologically significant homology to each other despite having apparently low sequence identity. Homologous nucleic acid sequences are considered to be those that will hybridise to each other under conditions of low stringency (M. R. Green, J. Sambrook, 2012, Molecular Cloning: A Laboratory Manual, Fourth Edition, Books 1-3, Cold Spring Harbor Laboratory Press, Cold Spring Harbor, NY).

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally occurring amino acids are also well known in the art. For instance, non-naturally occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e., non-naturally occurring) analogues of those specific amino acids. They may also be produced by naked ligation if the mutant monomer is produced using partial peptide synthesis. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid.

Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged |
| | | | (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged (+) | Trp | aromatic, hydrophobic, |
| | | | neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

Proteins can also be fusion proteins, referring in particular to genetic fusion, made e.g., by recombinant DNA technology. Proteins can also be conjugated, or “conjugated to”, as used herein, which refers, in particular, to chemical and/or enzymatic conjugation resulting in a stable covalent link. For example, two, more or all of the polypeptide subunits of a multimeric auxiliary protein and/or nanopore may be fused, and/or a polypeptide subunit of an auxiliary protein may be fused to a monomer of the nanopore.

Proteins may form a protein complex when several polypeptides or protein monomers bind to or interact with each other. “Binding” means any interaction, be it direct or indirect. A direct interaction implies a contact between the binding partners, for instance through a covalent link or coupling. An indirect interaction means any interaction whereby the interaction partners interact in a complex of more than two compounds. The interaction can be completely indirect, with the help of one or more bridging molecules, or partly indirect, where there is still a direct contact between the partners, which is stabilized by the additional interaction of one or more compounds. The “complex” as referred to in this disclosure is defined as a group of two or more associated proteins, which might have different functions. The association between the different polypeptides of the protein complex might be via non-covalent interactions, such as hydrophobic or ionic forces, or may as well be a covalent binding or coupling, such as disulphide bridges, or peptidic bonds. Covalent “binding” or “coupling” are used interchangeably herein and may also involve “cysteine coupling” or “reactive or photoreactive amino acid coupling”, referring to a bioconjugation between cysteines or between (photo)reactive amino acids, respectively, which is a chemical covalent link to form a stable complex. Examples of photoreactive amino acids include azidohomoalanine, homopropargylglycine, homoallelglycine, p-acetyl-Phe, p-azido-Phe, p-propargyloxy-Phe, and p-benzoyl-Phe (Wang et al. 2012, in Protein Engineering, DOI: 10.5772/28719; Chin et al. 2002, Proc. Nat. Acad. Sci. USA 99(17); 11020-24).

A “transmembrane protein pore” or “biological pore” is a transmembrane protein structure defining a channel or hole that allows the translocation of molecules and ions from one side of the membrane to the other. The translocation of ionic species through the pore may be driven by an electrical potential difference applied to either side of the pore. A “nanopore” is a pore in which the minimum diameter of the channel through which molecules or ions pass is in the order of nanometres (10⁻⁹ metres). The minimum diameter is the diameter at the narrowest point of the constriction. The transmembrane protein pore may be monomeric or oligomeric in nature.

Typically, the pore comprises a plurality of polypeptide subunits arranged around a central axis thereby forming a protein-lined channel that extends substantially perpendicular to the membrane in which the nanopore resides. The number of polypeptide subunits is not limited.

Typically, the number of subunits is from 5 to up to 30, suitably the number of subunits is from 6 to 10. Alternatively, the number of subunits is not defined as in the case of perfringolysin or related large membrane pores. The portions of the protein subunits within the nanopore that form protein-lined channel typically comprise secondary structural motifs that may include one or more trans-membrane β-barrel, and/or α-helix sections.

The monomer, pore or transmembrane protein pore of the disclosure is suited for analyte characterization, especially the characterization of polypeptides or polynucleotides. In some embodiments, the monomer, pore, or transmembrane protein pore described herein can be used for sequencing polynucleotide sequences e.g., because it can discriminate between different nucleotides with a high degree of sensitivity. In some embodiments, the monomer, pore, or transmembrane protein pore described herein can be used for sequencing polypeptide sequences e.g., because it can discriminate between different amino acids with a high degree of sensitivity. The monomer, pore or transmembrane protein pore of the disclosure may be an isolated, substantially isolated, purified, or substantially purified. A monomer, pore or transmembrane protein pore of the disclosure is “isolated” or purified if it is completely free of any other components, such as lipids and/or other pores, or other proteins with which it is normally associated in its native state, or if it is sufficiently enriched from a membranous compartment. A monomer, pore or transmembrane protein pore is substantially isolated if it is mixed with carriers or diluents which will not interfere with its intended use. For instance, a monomer, pore, or transmembrane protein pore is substantially isolated or substantially purified if it is present in a form that comprises less than 10%, less than 5%, less than 2% or less than 1% of other components, such as triblock copolymers, lipids, or other pores. Alternatively, a monomer, pore or transmembrane protein pore of the disclosure may be a transmembrane pore, when present in a membrane.

The “constriction”, “orifice”, “constriction region”, “channel constriction”, “constriction site”, or “reader head” as used interchangeably herein, refers to an aperture defined by a luminal surface of a pore, which acts to allow the passage of ions and target molecules (e.g., but not limited to amino acids, polypeptides, polynucleotides or individual nucleotides) but no other non-target molecules through the pore channel or continuous channel formed by the pore and auxiliary protein or peptide. In some embodiments, the constriction(s) are the narrowest aperture(s) within a pore. In this embodiment, the constriction(s) may serve to limit the passage of molecules through the pore. The size of the constriction is typically a key factor in determining suitability of a nanopore for nucleic acid sequencing applications. If the constriction is too small, the molecule to be sequenced will not be able to pass through. However, to achieve a maximal effect on ion flow through the channel, the constriction should not be too large. For example, the constriction should preferably not be wider than the solvent-accessible transverse diameter of a target analyte. Ideally, any constriction should be as close as possible in diameter to the transverse diameter of the analyte passing through. For sequencing of nucleic acids and nucleic acid bases, suitable constriction diameters are in the nanometre range (10⁻⁹-meter range).

Suitably, the diameter should be in the region of 0.5 to 2.0 nm, or 0.5 to 4.0 nm, typically, the diameter is in the region of 0.7 to 1.2 nm, such as 0.9 nm (9 Å). Such diameters may be particularly suited for sequencing of single-stranded nucleic acids. Larger diameters, such as from about 1.2 nm to about 4 nm, such as about 2 to about 4 nm or about 3 nm to about 4 nm may be particularly suited for sequencing of double-stranded nucleic acids.

When two or more constrictions are present and spaced apart each constriction may interact with or “read” separate nucleotides within the nucleic acid strand at the same time or separate amino acids within the polypeptide at the same time. In this situation, the reduction in ion flow through the channel will be the result of the combined restriction in flow of all the constrictions containing nucleotides or amino acids. Hence, in some instances a double constriction may lead to a composite current signal. In certain circumstances, the current read-out for one constriction, or “reading head”, may not be able to be determined individually when two such reading heads are present. Suitable distances between constriction regions are discussed below. The pore or transmembrane pore of the invention includes pores or transmembrane pores with two or more reader heads, meaning, two or more channel constrictions positioned in such a way to provide a suitable separate reader head without interfering the accuracy of other constriction channel reader heads.

A constriction region or constriction site may be formed by one or more specific amino acid residues within the protein sequence of a transmembrane protein nanopore and/or an auxiliary protein or peptide.

In wild type PorARr, PorBRr and PorARc, the constriction is located towards the bottom of the pore. This is described as part (c) below.

### PorARc Pores and Monomers of the Invention

The present invention provides a mutant Rhodococcus pore comprising a monomer which is a variant of the sequence shown in SEQ ID NO: 2, wherein the variant comprises one or more mutations between positions 9 and 200 of SEQ ID NO: 2 and wherein the one or more mutations alter the ability of the pore to interact with a target analyte. Any of the specific embodiments discussed below apply to this embodiment of the invention. The variant preferably comprises one or more mutations between positions 10 and 190 or 11 and 180 of SEQ ID NO: 2. The variant preferably comprises one or more mutations between positions 87 and 104.

The present invention also provides a mutant Rhodococcus pore comprising a monomer which is a variant of the sequence shown in SEQ ID NO: 2, wherein the variant comprises one or more mutations at one or more of the positions in (a), (b) and/or (c) below and wherein the one or more mutations alter the ability of the pore to interact with a target analyte.

The target analyte may be any of those discussed below. The target analyte is preferably an amino acid, a polypeptide, a nucleotide, or a polynucleotide. The ability of the pore to interact with the target analyte may be measured using known methods, including those described in the Example. The pore may comprise any number of monomers, such as 2, 3, 4, 5, 6, 7, 8, 9 or 10 monomers. Any number of the monomers in the pore may be variants as defined above. The pore may be homo-oligomeric or hetero-oligomeric as defined above.

The present invention also provides a mutant Rhodococcus porin monomer comprising a variant of the sequence shown in SEQ ID NO: 2, wherein the variant comprises one or more mutations between positions 9 and 200 of SEQ ID NO: 2, wherein the one or more mutations alter the ability of the monomer to interact with a target analyte and wherein the monomer can form a pore. Any of the specific embodiments discussed below apply to this embodiment of the invention. The variant preferably comprises one or more mutations between positions 10 and 190 or 11 and 180 of SEQ ID NO: 2. The variant preferably comprises one or more mutations between positions 87 and 104.

The invention also provides a mutant Rhodococcus porin monomer comprising a variant of the sequence shown in SEQ ID NO: 2. The variant comprises one or more mutations at one or more positions in (a), (b) and/or (c) below, the one or more mutations alter the ability of the monomer to interact with a target analyte and the monomer can form a pore.

The target analyte is preferably an amino acid, a polypeptide, a nucleotide, or a polynucleotide.

The ability of the monomer to interact with the target analyte may be measured using known methods, including those described in the Example. The ability of the monomer to form a pore may be determined using known methods, including those in the Example.

The mutant monomers have improved target analyte reading properties i.e., display improved analyte capture and discrimination. In particular, pores constructed from the mutant monomers capture amino acids, polypeptides, nucleotides, and polynucleotides more easily than the wild type. In addition, pores constructed from the mutant monomers display an increased current range, which makes it easier to discriminate between different amino acids or nucleotides, and a reduced variance of states, which increases the signal-to-noise ratio. In addition, the number of amino acids or nucleotides contributing to the current as the nucleic acid moves through pores constructed from the mutants is decreased. This makes it easier to identify a direct relationship between the observed current as the polypeptide or polynucleotide moves through the pore and the polypeptide or polynucleotide sequence. The improved amino acid or nucleotide reading properties of the mutants are achieved via five main mechanisms, namely by changes in the: sterics (increasing or decreasing the size of amino acid residues); charge (e.g., introducing +ve charge to interact with the nucleic acid sequence); hydrogen bonding (e.g., introducing amino acids that can hydrogen bond to the base pairs); pi stacking (e.g., introducing amino acids that interact through delocalised electron pi systems); and/or alteration of the structure of the pore (e.g., introducing amino acids that increase the size of the vestibule and/or constriction).

Any one or more of these five mechanisms may be responsible for the improved properties of the pores of the invention. For instance, a pore of the invention may display improved amino acid or nucleotide reading properties as a result of altered sterics, altered hydrogen bonding and an altered structure.

The introduction of bulky residues, such as phenylalanine (F), tryptophan (W), tyrosine (Y) or histidine (H), increases the sterics of the pore. The introduction of aromatic residues, such as phenylalanine (F), tryptophan (W), tyrosine (Y) or histidine (H), also increases the pi staking in the pore. The introduction of bulky or aromatic residues also alters the structure of the pore, for instance by opening up the pore and increasing the size of the vestibule and/or constriction. This is described in more detail below.

The one or more positions occur in three regions of the monomer: the cap, the middle, or the bottom.

In the cap (a), the positions are D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y 159, Q161, V163, S164, D165, N166, G167, D168, V169, and T171.

In the middle (b), the positions are S74, G76, E78, T80, D82, T84, Q108, G110, G112, T114, E116, and P117.

In the bottom (c), the positions are G87, E89, G91, S93, S94, A95, G96, A97, S99, T101, S103, and D104.

The variant may comprise one or more mutations at one or more of the positions in any combination of the cap, middle and bottom, namely in (a), (b), (c), (a) and (b), (b) and (c), (a) and (c), or (a), (b) and (c).

The variant may comprise any number of one or more mutations, such as 1, 2, 3, 4, 5, 6, 7, 8, 9, 10, 11, 12, 13, 14, 15, 16, 17, 18, 19, 20 or more mutations.

The variant preferably comprises one or more mutations at least at the following positions of SEQ ID NO: 2:
E78/D82/E89,
E78/D82/E89/E116/E125/D165,
E57/E78/D82/E89/E116/E125/D165,
D55/E57/D58/E78/D82/E89/E116/E125/D165,
E78/D82/E89/D104/E116/E122/E125/D165,
D55/E57/D58/E78/D82/E89/D104/E116/E122/E125/D165,
E57/E78/D82/E89/D104/E116/E122/E125/D165,
E78/D82/E89/T101/E116/E125/D165,
E78/D82/G87/E89/E116/E125/D165,
E78/D82/E89/G91/I92/E116/E125/D165,
E78/D82/E89/I92/E116/E125/D165,
E78/D82/E89/S99/E116/E125/D165,
E78/D82/E89/S103/E116/E125/D165,
E78/D82/E89/D104/E116/E125/D165,
E78/D82/E89/E116/E122/E125/D165,
E78/D82/E89/G91/I92/E116/E125/D165, or
E78/D82/E89/E116/E125/D165.

The variant preferably comprises or consists of mutations at the immediately above positions of SEQ ID NO: 2.

The one or more mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position. The one or more mutations are one or more substitutions. The skilled person is capable of making suitable substitutions based on Tables 1 and 2 above.

In the cap (a), mutations are preferably made to include positive, polar, or hydrophobic amino acids in order to either reduce negativity (so that the capture of negatively charged analytes is promoted) or improve/stabilise the interaction with the enzyme. Amino acids at positions in (a) may be substituted with R, K, S, T, N, Q, G, A, V, L, I, P, C, F, Y or W.

In the middle (b), mutations are preferably made to include positive, polar, or hydrophobic amino acids in order to reduce negativity (so that the capture of negatively charged analytes is promoted). Amino acids at positions in (b) may be substituted with R, K, S, T, N, Q, G, A, V, L, I, P, C, F, Y or W.

In the bottom (c), mutations are preferably made to include polar, hydrophobic, positive, or negative in order to change the discrimination (residues in the middle/cap may also influence discrimination, but likely to a lesser extent). Amino acids at positions in (c) may be substituted with S, T, N, Q, G, A, V, L, I, P, C, R, K, D, E, F, Y, or W.

Mutate to polar, hydrophobic, positive, or negative in order to change the discrimination (residues in the middle/cap may also influence discrimination, but likely to a lesser extent)

The one or more mutations are preferably selected from the following specific substitutions:
D to Q, S or Y, more preferably D to S,
G to S, N, Q, T or A, more preferably G to S or N,
N to S, Q, L, Y, M, I, V or G,
T to V, S, N, Q, R, F or I, more preferably T to V, S, N, Q or R,
P to S,
A to R, V, D, N, S or T,
E to S, N, T, A, R, G, V, L, I, F, Q or K,
F to R, K, S, T, N, Q, G, A, V, L, I, P, C, Y or W,
S to N or C, more preferably S to N,
Y to R, K, S, T, N, Q, G, A, V, L, I, P, C, D, E, F, or W,
Q to R,
V to I,
I to G, L, S, F, V, A, P, W, Q, N, C, S, Y, K or T, more preferably I to N
E to R,
H to F, N, Q or A,
L to N, S, Y, V, Q, T, R, A, G, C, F or D, and
K to R, S, T, N, Q, G, A, V, L, I, P, C, D, E, F, Y or W.

The variant preferably comprises at least the following mutations in SEQ ID NO: 2:
E78S/D82S/E89N,
E78S/D82S/E89N/E116T/E125A/D165S,
E78S/D82S/E89R,
E78R/D82S/E89R,
E78R/D82S/E89R/E116T/E125A/D165S,
E57R/E78R/D82S/E89R/E116T/E125A/D165S,
D55S/E57S/D58S/E78R/D82S/E89R/E116T/E125A/D165S,
E78R/D82S/E89R/D104S/E116T/E122S/E125A/D165S,
D55S/E57S/D58S/E78R/D82S/E89R/D104S/E116T/E122S/E125A/D165S,
E57R/E78R/D82S/E89R/D104S/E116T/E122S/E125A/D165S,
E78R/D82S/E89G/E116T/E125A/D165S,
E78R/D82S/E89A/E116T/E125A/D165S,
E78R/D82S/E89V/E116T/E125A/D165S,
E78R/D82S/E89L/116T/E125A/D165S,
E78R/D82S/E891/E116T/E125A/D165S,
E78R/D82S/E89F/E116T/E125A/D165S,
E78R/D82S/E89S/E116T/E125A/D165S,
E78R/D82S/E89T/E116T/E125A/D165S,
E78R/D82S/E89Q/E116T/E125A/D165S,
E78R/D82S/E89K/E116T/E125A/D165S,
E78R/D82S/E89R/T101V/E116T/E125A/D165S,
E78R/D82S/E89R/T101S/E116T/E125A/D165S,
E78R/D82S/E89R/T101N/E116T/E125A/D165S,
E78R/D82S/E89R/T101Q/E116T/E125A/D165S,
E78R/D82S/E89R/T101R/E116T/E125A/D165S,
E78R/D82S/E89N/101V/E116T/E125A/D165S,
E78R/D82S/E89N/101S/E 16T/E125A/D165S,
E78R/D82S/E89N/101N/E 16T/E125A/D165S,
E78R/D82S/E89N/101Q/E 16T/E125A/D165S,
E78R/D82S/E89N/101R/E 16T/E125A/D165S,
E78R/D82S/G87S/E89R/E116T/E125A/D165S,
E78R/D82S/E89R/G91S/E116T/E125A/D165S,
E78R/D82S/E89R/G91N/E116T/E125A/D165S,
E78R/D82S/E89R/G91N/I92N/E116T/E125A/D165S,
E78R/D82S/E89R/I92N/E116T/E125A/D165S,
E78R/D82S/E89R/S99N/E116T/E125A/D165S,
E78R/D82S/E89R/S103N/E116T/E125A/D165S,
E78R/D82S/E89R/D104S/E116T/E125A/D165S,
E78R/D82S/E89R/E116T/E122S/E125A/D165S,
E78G/D82S/E89L/G91N/I92N/E116G/E125A/D165S, or
E78G/D82S/E89L/E116G/E125A/D165S.

The variant preferably comprises or consists of the mutations shown immediately above for SEQ ID NO: 2. The variant may be any of the mutants shown in Table 3.

The variant of SEQ ID NO: 2 is preferably not identical to SEQ ID NO: 4 or 6 (i.e., does not share 100% amino acid identity to SEQ ID NO: 4 or 6).

### PorARr Pores and Monomers of the Invention

The present invention also provides a mutant Rhodococcus pore comprising a monomer which is a variant of the sequence shown in SEQ ID NO: 4, wherein the variant comprises one or more mutations between positions 9 and 250 of SEQ ID NO: 4 and wherein the one or more mutations alter the ability of the pore to interact with a target analyte. Any of the specific embodiments discussed below apply to this embodiment of the invention. The variant preferably comprises one or more mutations between positions 10 and 200 or 11 and 180 of SEQ ID NO: 4. The variant preferably comprises one or more mutations between positions 87 and 158.

The present invention also provides a mutant Rhodococcus pore comprising a monomer which is a variant of the sequence shown in SEQ ID NO: 4, wherein the variant comprises one or more mutations at one or more of the positions in (a), (b) and/or (c) below and wherein the one or more mutations alter the ability of the pore to interact with a target analyte.

The target analyte may be any of those discussed below. The target analyte is preferably an amino acid, a polypeptide, a nucleotide, or a polynucleotide. The ability of the pore to interact with the target analyte may be measured using known methods, including those described in the Example. The pore may comprise any number of monomers, such as 2, 3, 4, 5, 6, 7, 8, 9 or 10 monomers. Any number of the monomers in the pore may be variants as defined above. The pore may be homo-oligomeric or hetero-oligomeric as defined above.

The present invention provides a mutant Rhodococcus porin monomer comprising a variant of the sequence shown in SEQ ID NO: 4, wherein the variant comprises one or more mutations between positions 9 and 250 of SEQ ID NO: 4, wherein the one or more mutations alter the ability of the monomer to interact with a target analyte and wherein the monomer can form a pore. Any of the specific embodiments discussed below apply to this embodiment of the invention. The variant preferably comprises one or more mutations between positions 10 and 200 or 11 and 180 of SEQ ID NO: 4. The variant preferably comprises one or more mutations between positions 87 and 158.

The invention also provides a mutant Rhodococcus porin monomer comprising a variant of the sequence shown in SEQ ID NO: 4. The variant comprises one or more mutations at one or more of the positions in (a), (b) and/or (c) below, the one or more mutations alter the ability of the monomer to interact with a nucleotide and the monomer can form a pore.

The target analyte is preferably an amino acid, a polypeptide, a nucleotide, or a polynucleotide. The ability of the monomer to interact with the target analyte may be measured using known methods, including those described in the Example. The ability of the monomer to form a pore may be determined using known methods, including those in the Example.

The mutant monomers based on SEQ ID NO: 4 have improved target analyte reading properties for the same reasons as discussed above for SEQ ID NO: 2.

The one or more positions occur in three regions of the monomer: the cap, the middle, or the bottom.

In the cap (a), the positions are S11, D12, G13, H14, V51, G52, P53, D54, A55, A56, D57, F58, E59, G60, S61, Q64, Y67, Q68, F69, W71, P173, G174, I175, E176, E177, L178, V179, V180, E182, G183, E184, F185, D186, G187, D188, F189, V197, H198, A200, T202, G203, F213, R215, I217, T218, A219, N220, G221, D222, N223, and T225.

In the middle (b), the positions are S74, D76, S78, G80, A82, S84, Q162, T164, G166, E168, T170, and P171.

In the bottom (c), the positions are Q87, E89, E91, V92, A93, P94, G95, E96, V97, Y98, E99, P100, A101, T102, E103, T104, K105, T106, V107, D108, G109, K110, E111, T112, E113, V114, P115, V116, T117, A118, P119, V120, L121, N122, E123, D124, G125, S126, P127, I128, L129, D130, A131, D132, G133, K134, P135, V136, T137, E138, Q139, L140, Y141, R142, I143, K144, P145, D146, A147, T148, A149, T150, E151, N152, T153, T155, V156, G157, and G158.

The variant may comprise one or more mutations at one or more of the positions in any combination of the cap, middle and bottom, namely in (a), (b), (c), (a) and (b), (b) and (c), (a) and (c), or (a), (b) and (c).

The variant may comprise any number of one or more mutations, such as 1, 2, 3, 4, 5, 6, 7, 8, 9, 10, 11, 12, 13, 14, 15, 16, 17, 18, 19, 20 or more mutations.

The variant preferably comprises one or more mutations at least at the following positions of SEQ ID NO: 4:
E182/E184,
E89/E91,
E89/E91/E168/E182/E184, or
E89/E91/E96/E99/E 113/E123/D130/E182/E184.

The variant preferably comprises or consists of mutations at the positions shown immediately above for SEQ ID NO: 4.

The one or more mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position. The one or more mutations are one or more substitutions. The skilled person is capable of making suitable substitutions based on Tables 1 and 2 above.

The one or more mutations are preferably selected from the preferred or specific substitutions set out above for SEQ ID NO: 2.

The variant preferably comprises at least the following mutations in SEQ ID NO: 4:
E182R/E184R,
E89N/E91N,
E89N/E91N/E168R/E182R/E184R,
E89N/E91N/E96R/E99N/E113N/E123N/D130N/E182R/E184R, or
E89N/E91N/E96R/E99R/E113K/E123K/D130K/E182R/E184R.

The variant preferably comprises or consists of the mutations shown immediately above for SEQ ID NO: 4. The variant may be any of the mutants shown in Table 3.

The variant of SEQ ID NO: 4 is preferably not identical to SEQ ID NO: 2 or 6 (i.e., does not share 100% amino acid identity to SEQ ID NO: 2 or 6).

### PorBRr Pores and Monomers of the Invention

The present invention also provides a mutant Rhodococcus pore comprising a monomer which is a variant of the sequence shown in SEQ ID NO: 6, wherein the variant comprises one or more mutations between positions 9 and 200 of SEQ ID NO: 6 and wherein the one or more mutations alter the ability of the pore to interact with a target analyte. Any of the specific embodiments discussed below apply to this embodiment of the invention. The variant preferably comprises one or more mutations between positions 10 and 190 or 11 and 180 of SEQ ID NO: 6. The variant preferably comprises one or more mutations between positions 88 and 105.

The present invention also provides a mutant Rhodococcus pore comprising a monomer which is a variant of the sequence shown in SEQ ID NO: 6, wherein the variant comprises one or more mutations at one or more of the positions in (a), (b) and/or (c) below and wherein the one or more mutations alter the ability of the pore to interact with a target analyte.

The target analyte may be any of those discussed below. The target analyte is preferably an amino acid, a polypeptide, a nucleotide, or a polynucleotide. The ability of the pore to interact with the target analyte may be measured using known methods, including those described in the Example. The pore may comprise any number of monomers, such as 2, 3, 4, 5, 6, 7, 8, 9 or 10 monomers. Any number of the monomers in the pore may be variants as defined above. The pore may be homo-oligomeric or hetero-oligomeric as defined above.

The present invention provides a mutant Rhodococcus porin monomer comprising a variant of the sequence shown in SEQ ID NO: 6, wherein the variant comprises one or more mutations between positions 9 and 200 of SEQ ID NO: 6, wherein the one or more mutations alter the ability of the monomer to interact with a nucleotide and wherein the monomer can form a pore.

Any of the specific embodiments discussed below apply to this embodiment of the invention.

The variant preferably comprises one or more mutations between positions 10 and 190 or 11 and 180 of SEQ ID NO: 6. The variant preferably comprises one or more mutations between positions 88 and 105.

The invention also provides a mutant Rhodococcus porin monomer comprising a variant of the sequence shown in SEQ ID NO: 6. The variant comprises one or more mutations at one or more of the positions in (a), (b) and/or (c) below, the one or more mutations alter the ability of the monomer to interact with a nucleotide and the monomer can form a pore.

The target analyte is preferably an amino acid, a polypeptide, a nucleotide, or a polynucleotide.

The ability of the monomer to interact with the target analyte may be measured using known methods, including those described in the Example. The ability of the monomer to form a pore may be determined using known methods, including those in the Example.

The mutant monomers based on SEQ ID NO: 6 have improved target analyte reading properties for the same reasons as discussed above for SEQ ID NO: 2.

The one more or positions occur in three regions of the monomer: the cap, the middle, or the bottom.

In the cap (a), the positions are S11, T12, D13, G14, Y15, V52, G53, A54, D55, A56, A57, D58, A59, E60, G61, T62, Q65, Y68, Q69, F70, W72, P120, G121, I122, E123, E124, L125, V126, V127, E129, G130, E131, F132, D133, G134, D135, F136, V144, H145, T147, S149, G150, F160, R162, I164, T165, A166, N167, G168, D169, N170, and T172.

In the middle (b), the positions are S75, D77, A79, G81, T83, D85, Q109, Y111, E113, E115, T117, and P118.

In the bottom (c), the positions are G88, E90, E92, L93, S94, A95, K96, D97, G98, P99, T100, T102, V103, T104, and D105.

The variant may comprise one or more mutations at one or more of the positions in any combination of the cap, middle and bottom, namely in (a), (b), (c), (a) and (b), (b) and (c), (a) and (c), or (a), (b) and (c).

The variant may comprise any number of one or more mutations, such as 1, 2, 3, 4, 5, 6, 7, 8, 9, 10, 11, 12, 13, 14, 15, 16, 17, 18, 19, 20 or more mutations.

The variant preferably comprises one or more mutations at least at the following positions of SEQ ID NO: 6:
E90,
E92,
E90/E92,
E90/E92/E115/E131, or
E90/E92/E113/E115/E129/E131.

The variant preferably comprises one or more mutations at least at the following positions of SEQ ID NO: 6:
E90/E92/E115/E131,
E90/E92/T100/E115/E131,
E90/E92/T102/E115/E131,
E90/E92/T104/E115/E131,
E90/E92/D105/E115/E131,
E90/E92/T102/T104/E115/E131, or
E90/E92/E113/E115/E129/E131.

The variant preferably comprises one or more mutations at least at the following positions of SEQ ID NO: 6:
E90,
E92,
E90/E92,
E90/E92/E115/E131,
E90/E92/T100/E115/E131,
E90/E92/T102/E115/E131,
E90/E92/T104/E115/E131,
E90/E92/D105/E115/E131,
E90/E92/T102/T104/E115/E131, or
E90/E92/E113/E115/E129/E131.

The variant preferably comprises or consists of mutations at the positions shown immediately above for SEQ ID NO: 6.

The one or more mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position. The one or more mutations are one or more substitutions. The skilled person is capable of making suitable substitutions based on Tables 1 and 2 above.

The one or more mutations are preferably selected from the preferred or specific substitutions set out above for SEQ ID NO: 2. The E at position 90 is preferably substituted with G, A, V, L, I, F, Y, S, T, Q, N, C or P. The E at position 90 is preferably substituted with N, G, V, L or I. The E at position 90 is preferably substituted with N (E90N). The E at position 92 is preferably substituted with N, G, A, V, L, I, F, Y, S, T, Q, C or P. The E at position 92 is preferably substituted with N, A, V, L or I. The E at position 92 is preferably substituted with N (E92N).

The T at position 100 is preferably substituted with A, V, N or Q. The T at position at 100 is preferably substituted with N (T100N). The T at position 102 is preferably substituted with A, V, N or Q. The T at position 102 is substituted with V or N. The T at position 104 is preferably substituted with A, V, N or Q. The T at position 104 is substituted with A or N. The D at position 105 is preferably substituted with A. The E at position 113 is preferably substituted with R (E113R) or K (E113K). The E at position 113 is preferably substituted with R (E113R). The E at position 115 is preferably substituted with R (E115R) or K (E115K). The E at position 115 is preferably substituted with R (E115R). The E at position 131 is preferably substituted with R (E131R) or K (E131K). The E at position 131 is preferably substituted with R (E131R).

The variant preferably comprises at least the following mutations in SEQ ID NO: 6:
E90N,
E92N,
E90N/E92N,
E90N/E92N/E115R/E131R, or
E90N/E92N/E113R/E115R/E129R/E131R.

The variant preferably comprises at least the following mutations in SEQ ID NO: 6:
E90C,
E90P,
E92C,
E92P,
E90C/E92C,
E90P/E92P,
E90C/E92P,
E90P/E92C,
E90N/E92C,
E90N/E92P,
E90C/E92N,
E90P/E92N,
E90C/E92C/E115R/E131R,
E90C/E92P/E115R/E131R,
E90P/E92C/E115R/E131R,
E90C/E92C/E115R/E131R,
E90N/E92C/E115R/E131R,
E90N/E92P/E115R/E131R,
E90P/E92N/E115R/E131R,
E90C/E92N/E115R/E131R,
E90C/E92C/E113R/E115R/E129R/E131R,
E90C/E92P/E 113R/E115R/E129R/E131R,
E90P/E92C/E113R/E115R/E129R/E131R,
E90P/E92P/E113R/E115R/E129R/E131R,
E90C/E92N/E 113R/E115R/E129R/E131R,
E90P/E92N/E113R/E115R/E129R/E131R,
E90N/E92C/E113R/E115R/E129R/E131R,
E90N/E92P/E113R/E115R/E129R/E131R,
E90C,
E90P,
E92C,
E92P,
E90C/E92C,
E90P/E92P,
E90C/E92P,
E90P/E92C,
E90N/E92C,
E90N/E92P,
E90C/E92N,
E90P/E92N,
E90C/E92C/E115K/E131K,
E90C/E92P/E115K/E131K,
E90P/E92C/E115K/E131K,
E90C/E92C/E115K/E131K,
E90N/E92C/E115K/E131K,
E90N/E92P/E115K/E131K,
E90P/E92N/E115K/E131K,
E90C/E92N/E115K/E131K,
E90C/E92C/E113K/E115K/E129K/E131K,
E90C/E92P/E113K/E115K/E129K/E131K,
E90P/E92C/E113K/E115K/E129K/E131K,
E90P/E92P/E113K/E115K/E129K/E131K,
E90C/E92N/E113K/E115K/E129K/E131K,
E90P/E92N/E113K/E115K/E129K/E131K,
E90N/E92C/E113K/E115K/E129K/E131K,
E90N/E92P/E113K/E115K/E129K/E131K,
E90G/E92N/E115R/E131R,
E90A/E92N/E115R/E131R,
E90V/E92N/E115R/E131R,
E90L/E92N/E115R/E131R,
E90I/E92N/E115R/E131R,
E90F/E92N/E115R/E131R,
E90Y/E92N/E115R/E131R,
E90S/E92N/E115R/E131R,
E90T/E92N/E115R/E131R,
E90Q/E92N/E115R/E131R,
E90C/E92N/E115R/E131R,
E90P/E92N/E115R/E131R,
E90N/E92G/E115R/E131R,
E90N/E92A/E115R/E131R,
E90N/E92V/E115R/E131R,
E90N/E92L/E115R/E131R,
E90N/E92I/E115R/E131R,
E90N/E92F/E115R/E131R,
E90N/E92Y/E115R/E131R,
E90N/E92S/E115R/E131R,
E90N/E92T/E115R/E131R,
E90N/E92Q/E115R/E131R,
E90N/E92N/E115R/E131R,
E90N/E92C/E115R/E131R,
E90N/E92P/E115R/E131R,
E90C/E92C/E115R/E131R,
E90P/E92P/E115R/E131R,
E90C/E92P/E115R/E131R,
E90P/E92C/E115R/E131R,
E90N/E92N/T100N/E115R/E131R,
E90N/E92N/T102A/E115R/E131R,
E90N/E92N/T102V/E115R/E131R,
E90N/E92N/T102N/E115R/E131R,
E90N/E92N/T102Q/E115R/E131R,
E90N/E92N/T104A/E115R/E131R,
E90N/E92N/T104N/E115R/E131R,
E90N/E92N/D105A/E115R/E131R,
E90N/E92N/T102V/T104N/E115R/E131R,
E90N/E92N/T102N/T104N/E115R/E131R,
E90N/E92N/E113R/E115R/E129R/E131R,
E90C/E92N/T100N/E115R/E131R,
E90C/E92N/T102A/E115R/E131R,
E90C/E92N/T102V/E115R/E131R,
E90C/E92N/T102N/E115R/E131R,
E90C/E92N/T102Q/E115R/E131R,
E90C/E92N/T104A/E115R/E131R,
E90C/E92N/T104N/E115R/E131R,
E90C/E92N/D105A/E115R/E131R,
E90C/E92N/T102V/T104N/E115R/E131R,
E90C/E92N/T102N/T104N/E115R/E131R,
E90C/E92N/E113R/E115R/E129R/E131R,
E90N/E92C/T100N/E115R/E131R,
E90N/E92C/T102A/E115R/E131R,
E90N/E92C/T102V/E115R/E131R,
E90N/E92C/T102N/E115R/E131R,
E90N/E92C/T102Q/E115R/E131R,
E90N/E92C/T104A/E115R/E131R,
E90N/E92C/T104N/E115R/E131R,
E90N/E92C/D105A/E115R/E131R,
E90N/E92C/T102V/T104N/E115R/E131R,
E90N/E92C/T102N/T104N/E115R/E131R,
E90N/E92C/E113R/E115R/E129R/E131R,
E90C/E92C/T100N/E115R/E131R,
E90C/E92C/T102A/E115R/E131R,
E90C/E92C/T102V/E115R/E131R,
E90C/E92C/T102N/E115R/E131R,
E90C/E92C/T102Q/E115R/E131R,
E90C/E92C/T104A/E115R/E131R,
E90C/E92C/T104N/E115R/E131R,
E90C/E92C/D105A/E115R/E131R,
E90C/E92C/T102V/T104N/E115R/E131R,
E90C/E92C/T102N/T104N/E115R/E131R,
E90C/E92C/E113R/E115R/E129R/E131R,
E90C/E92P/T100N/E115R/E131R,
E90C/E92P/T102A/E115R/E131R,
E90C/E92P/T102V/E115R/E131R,
E90C/E92P/T102N/E115R/E131R,
E90C/E92P/T102Q/E115R/E131R,
E90C/E92P/T104A/E115R/E131R,
E90C/E92P/T104N/E115R/E131R,
E90C/E92P/D105A/E115R/E131R,
E90C/E92P/T102V/T104N/E115R/E131R,
E90C/E92P/T102N/T104N/E115R/E131R,
E90C/E92P/E113R/E115R/E129R/E131R,
E90P/E92C/T100N/E115R/E131R,
E90P/E92C/T102A/E115R/E131R,
E90P/E92C/T102V/E115R/E131R,
E90P/E92C/T102N/E115R/E131R,
E90P/E92C/T102Q/E115R/E131R,
E90P/E92C/T104A/E115R/E131R,
E90P/E92C/T104N/E115R/E131R,
E90P/E92C/D105A/E115R/E131R,
E90P/E92C/T102V/T104N/E115R/E131R,
E90P/E92C/T102N/T104N/E115R/E131R,
E90P/E92C/E113R/E115R/E129R/E131R,
E90G/E92N/E115K/E131K,
E90A/E92N/E115K/E131K,
E90V/E92N/E115K/E131K,
E90I/E92N/E115K/E131K,
E90I/E92N/E115K/E131K,
E90F/E92N/E115K/E131K,
E90Y/E92N/E115K/E131K,
E90S/E92N/E115K/E131K,
E90T/E92N/E115K/E131K,
E90Q/E92N/E115K/E131K,
E90C/E92N/E115K/E131K,
E90P/E92N/E115K/E131K,
E90N/E92G/E115K/E131K,
E90N/E92A/E115K/E131K,
E90N/E92V/E115K/E131K,
E90N/E92L/E115K/E131K,
E90N/E92I/E115K/E131K,
E90N/E92F/E115K/E131K,
E90N/E92Y/E115K/E131K,
E90N/E92S/E115K/E131K,
E90N/E92T/E115K/E131K,
E90N/E92Q/E115K/E131K,
E90N/E92N/E115K/E131K,
E90N/E92C/E115K/E131K,
E90N/E92P/E115K/E131K,
E90C/E92C/E115K/E131K,
E90P/E92P/E115K/E131K,
E90C/E92P/E115K/E131K,
E90P/E92C/E115K/E131K,
E90N/E92N/T100N/E115K/E131K,
E90N/E92N/T102A/E115K/E131K,
E90N/E92N/T102V/E115K/E131K,
E90N/E92N/T102N/E115K/E131K,
E90N/E92N/T102Q/E115K/E131K,
E90N/E92N/T104A/E115K/E131K,
E90N/E92N/T104N/E115K/E131K,
E90N/E92N/D105A/E115K/E131K,
E90N/E92N/T102V/T104N/E115K/E131K,
E90N/E92N/T102N/T104N/E115K/E131K,
E90N/E92N/E113K/E115K/E129K/E131K,
E90C/E92N/T100N/E115K/E131K,
E90C/E92N/T102A/E115K/E131K,
E90C/E92N/T102V/E115K/E131K,
E90C/E92N/T102N/E115K/E131K,
E90C/E92N/T102Q/E115K/E131K,
E90C/E92N/T104A/E115K/E131K,
E90C/E92N/T104N/E115K/E131K,
E90C/E92N/D105A/E115K/E131K,
E90C/E92N/T102V/T104N/E115K/E131K,
E90C/E92N/T102N/T104N/E115K/E131K,
E90C/E92N/E113K/E115K/E129K/E131K,
E90N/E92C/T100N/E115K/E131K,
E90N/E92C/T102A/E115K/E131K,
E90N/E92C/T102V/E115K/E131K,
E90N/E92C/T102N/E115K/E131K,
E90N/E92C/T102Q/E115K/E131K,
E90N/E92C/T104A/E115K/E131K,
E90N/E92C/T104N/E115K/E131K,
E90N/E92C/D105A/E115K/E131K,
E90N/E92C/T102V/T104N/E115K/E131K,
E90N/E92C/T102N/T104N/E115K/E131K,
E90N/E92C/E113K/E115K/E129K/E131K,
E90C/E92C/T100N/E115K/E131K,
E90C/E92C/T102A/E115K/E131K,
E90C/E92C/T102V/E115K/E131K,
E90C/E92C/T102N/E115K/E131K,
E90C/E92C/T102Q/E115K/E131K,
E90C/E92C/T104A/E115K/E131K,
E90C/E92C/T104N/E115K/E131K,
E90C/E92C/D105A/E115K/E131K,
E90C/E92C/T102V/T104N/E115K/E131K,
E90C/E92C/T102N/T104N/E115K/E131K,
E90C/E92C/E113K/E115K/E129K/E131K,
E90C/E92P/T100N/E115K/E131K,
E90C/E92P/T102A/E115K/E131K,
E90C/E92P/T102V/E115K/E131K,
E90C/E92P/T102N/E115K/E131K,
E90C/E92P/T102Q/E115K/E131K,
E90C/E92P/T104A/E115K/E131K,
E90C/E92P/T104N/E115K/E131K,
E90C/E92P/D105A/E115K/E131K,
E90C/E92P/T102V/T104N/E115K/E131K,
E90C/E92P/T102N/T104N/E115K/E131K,
E90C/E92P/E113K/E115K/E129K/E131K,
E90P/E92C/T100N/E115K/E131K,
E90P/E92C/T102A/E115K/E131K,
E90P/E92C/T102V/E115K/E131K,
E90P/E92C/T102N/E115K/E131K,
E90P/E92C/T102Q/E115K/E131K,
E90P/E92C/T104A/E115K/E131K,
E90P/E92C/T104N/E115K/E131K,
E90P/E92C/D105A/E115K/E131K,
E90P/E92C/T102V/T104N/E115K/E131K,
E90P/E92C/T102N/T104N/E115K/E131K, or
E90P/E92C/E113K/E115K/E129K/E131K.

The variant preferably comprises at least the following mutations in SEQ ID NO: 6:
E90N,
E92N,
E90N/E92N,
E90N/E92N/E115R/E131R,
E90N/E92N/E113R/E115R/E129R/E131R,
E90C,
E90P,
E92C,
E92P,
E90C/E92C,
E90P/E92P,
E90C/E92P,
E90P/E92C,
E90N/E92C,
E90N/E92P,
E90C/E92N,
E90P/E92N,
E90C/E92C/E115R/E131R,
E90C/E92P/E115R/E131R,
E90P/E92C/E115R/E131R,
E90C/E92C/E115R/E131R,
E90N/E92C/E115R/E131R,
E90N/E92P/E115R/E131R,
E90P/E92N/E115R/E131R,
E90C/E92N/E115R/E131R,
E90C/E92C/E113R/E115R/E129R/E131R,
E90C/E92P/E113R/E115R/E129R/E131R,
E90P/E92C/E113R/E115R/E129R/E131R,
E90P/E92P/E113R/E115R/E129R/E131R,
E90C/E92N/E113R/E115R/E129R/E131R,
E90P/E92N/E113R/E115R/E129R/E131R,
E90N/E92C/E113R/E115R/E129R/E131R,
E90N/E92P/E113R/E115R/E129R/E131R,
E90C,
E90P,
E92C,
E92P,
E90C/E92C,
E90P/E92P,
E90C/E92P,
E90P/E92C,
E90N/E92C,
E90N/E92P,
E90C/E92N,
E90P/E92N,
E90C/E92C/E115K/E131K,
E90C/E92P/E115K/E131K,
E90P/E92C/E115K/E131K,
E90C/E92C/E115K/E131K,
E90N/E92C/E115K/E131K,
E90N/E92P/E115K/E131K,
E90P/E92N/E115K/E131K,
E90C/E92N/E115K/E131K,
E90C/E92C/E113K/E115K/E129K/E131K,
E90C/E92P/E113K/E115K/E129K/E131K,
E90P/E92C/E113K/E115K/E129K/E131K,
E90P/E92P/E113K/E115K/E129K/E131K,
E90C/E92N/E113K/E115K/E129K/E131K,
E90P/E92N/E113K/E115K/E129K/E131K,
E90N/E92C/E113K/E115K/E129K/E131K,
E90N/E92P/E113K/E115K/E129K/E131K,
E90G/E92N/E115R/E131R,
E90A/E92N/E115R/E131R,
E90V/E92N/E115R/E131R,
E90L/E92N/E115R/E131R,
E90I/E92N/E115R/E131R,
E90F/E92N/E115R/E131R,
E90Y/E92N/E115R/E131R,
E90S/E92N/E115R/E131R,
E90T/E92N/E115R/E131R,
E90Q/E92N/E115R/E131R,
E90C/E92N/E115R/E131R,
E90P/E92N/E115R/E131R,
E90N/E92G/E115R/E131R,
E90N/E92A/E115R/E131R,
E90N/E92V/E115R/E131R,
E90N/E92L/E115R/E131R,
E90N/E92I/E115R/E131R,
E90N/E92F/E115R/E131R,
E90N/E92Y/E115R/E131R,
E90N/E92S/E115R/E131R,
E90N/E92T/E115R/E131R,
E90N/E92Q/E115R/E131R,
E90N/E92C/E115R/E131R,
E90N/E92P/E115R/E131R,
E90C/E92C/E115R/E131R,
E90P/E92P/E115R/E131R,
E90C/E92P/E115R/E131R,
E90P/E92C/E115R/E131R,
E90N/E92N/T100N/E115R/E131R,
E90N/E92N/T102A/E115R/E131R,
E90N/E92N/T102V/E115R/E131R,
E90N/E92N/T102N/E115R/E131R,
E90N/E92N/T102Q/E115R/E131R,
E90N/E92N/T104A/E115R/E131R,
E90N/E92N/T104N/E115R/E131R,
E90N/E92N/D105A/E115R/E131R,
E90N/E92N/T102V/T104N/E115R/E131R,
E90N/E92N/T102N/T104N/E115R/E131R,
E90C/E92N/T100N/E115R/E131R,
E90C/E92N/T102A/E115R/E131R,
E90C/E92N/T102V/E115R/E131R,
E90C/E92N/T102N/E115R/E131R,
E90C/E92N/T102Q/E115R/E131R,
E90C/E92N/T104A/E115R/E131R,
E90C/E92N/T104N/E115R/E131R,
E90C/E92N/D105A/E115R/E131R,
E90C/E92N/T102V/T104N/E115R/E131R,
E90C/E92N/T102N/T104N/E115R/E131R,
E90C/E92N/E113R/E115R/E129R/E131R,
E90N/E92C/T100N/E115R/E131R,
E90N/E92C/T102A/E115R/E131R,
E90N/E92C/T102V/E115R/E131R,
E90N/E92C/T102N/E115R/E131R,
E90N/E92C/T102Q/E115R/E131R,
E90N/E92C/T104A/E115R/E131R,
E90N/E92C/T104N/E115R/E131R,
E90N/E92C/D105A/E115R/E131R,
E90N/E92C/T102V/T104N/E115R/E131R,
E90N/E92C/T102N/T104N/E115R/E131R,
E90N/E92C/E113R/E115R/E129R/E131R,
E90C/E92C/T100N/E115R/E131R,
E90C/E92C/T102A/E115R/E131R,
E90C/E92C/T102V/E115R/E131R,
E90C/E92C/T102N/E115R/E131R,
E90C/E92C/T102Q/E115R/E131R,
E90C/E92C/T104A/E115R/E131R,
E90C/E92C/T104N/E115R/E131R,
E90C/E92C/D105A/E115R/E131R,
E90C/E92C/T102V/T104N/E115R/E131R,
E90C/E92C/T102N/T104N/E115R/E131R,
E90C/E92C/E113R/E115R/E129R/E131R,
E90C/E92P/T100N/E115R/E131R,
E90C/E92P/T102A/E115R/E131R,
E90C/E92P/T102V/E115R/E131R,
E90C/E92P/T102N/E115R/E131R,
E90C/E92P/T102Q/E115R/E131R,
E90C/E92P/T104A/E115R/E131R,
E90C/E92P/T104N/E115R/E131R,
E90C/E92P/D105A/E115R/E131R,
E90C/E92P/T102V/T104N/E115R/E131R,
E90C/E92P/T102N/T104N/E115R/E131R,
E90C/E92P/E113R/E115R/E129R/E131R,
E90P/E92C/T100N/E115R/E131R,
E90P/E92C/T102A/E115R/E131R,
E90P/E92C/T102V/E115R/E131R,
E90P/E92C/T102N/E115R/E131R,
E90P/E92C/T102Q/E115R/E131R,
E90P/E92C/T104A/E115R/E131R,
E90P/E92C/T104N/E115R/E131R,
E90P/E92C/D105A/E115R/E131R,
E90P/E92C/T102V/T104N/E115R/E131R,
E90P/E92C/T102N/T104N/E115R/E131R,
E90P/E92C/E113R/E115R/E129R/E131R,
E90G/E92N/E115K/E131K,
E90A/E92N/E115K/E131K,
E90V/E92N/E115K/E131K,
E90I/E92N/E115K/E131K,
E90I/E92N/E115K/E131K,
E90F/E92N/E115K/E131K,
E90Y/E92N/E115K/E131K,
E90S/E92N/E115K/E131K,
E90T/E92N/E115K/E131K,
E90Q/E92N/E115K/E131K,
E90C/E92N/E115K/E131K,
E90P/E92N/E115K/E131K,
E90N/E92G/E115K/E131K,
E90N/E92A/E115K/E131K,
E90N/E92V/E115K/E131K,
E90N/E92L/E115K/E131K,
E90N/E92I/E115K/E131K,
E90N/E92F/E115K/E131K,
E90N/E92Y/E115K/E131K,
E90N/E92S/E115K/E131K,
E90N/E92T/E115K/E131K,
E90N/E92Q/E115K/E131K,
E90N/E92N/E115K/E131K,
E90N/E92C/E115K/E131K,
E90N/E92P/E115K/E131K,
E90C/E92C/E115K/E131K,
E90P/E92P/E115K/E131K,
E90C/E92P/E115K/E131K,
E90P/E92C/E115K/E131K,
E90N/E92N/T100N/E115K/E131K,
E90N/E92N/T102A/E115K/E131K,
E90N/E92N/T102V/E115K/E131K,
E90N/E92N/T102N/E115K/E131K,
E90N/E92N/T102Q/E115K/E131K,
E90N/E92N/T104A/E115K/E131K,
E90N/E92N/T104N/E115K/E131K,
E90N/E92N/D105A/E115K/E131K,
E90N/E92N/T102V/T104N/E115K/E131K,
E90N/E92N/T102N/T104N/E115K/E131K,
E90N/E92N/E113K/E115K/E129K/E131K,
E90C/E92N/T100N/E115K/E131K,
E90C/E92N/T102A/E115K/E131K,
E90C/E92N/T102V/E115K/E131K,
E90C/E92N/T102N/E115K/E131K,
E90C/E92N/T102Q/E115K/E131K,
E90C/E92N/T104A/E115K/E131K,
E90C/E92N/T104N/E115K/E131K,
E90C/E92N/D105A/E115K/E131K,
E90C/E92N/T102V/T104N/E115K/E131K,
E90C/E92N/T102N/T104N/E115K/E131K,
E90C/E92N/E113K/E115K/E129K/E131K,
E90N/E92C/T100N/E115K/E131K,
E90N/E92C/T102A/E115K/E131K,
E90N/E92C/T102V/E115K/E131K,
E90N/E92C/T102N/E115K/E131K,
E90N/E92C/T102Q/E115K/E131K,
E90N/E92C/T104A/E115K/E131K,
E90N/E92C/T104N/E115K/E131K,
E90N/E92C/D105A/E115K/E131K,
E90N/E92C/T102V/T104N/E115K/E131K,
E90N/E92C/T102N/T104N/E115K/E131K,
E90N/E92C/E113K/E115K/E129K/E131K,
E90C/E92C/T100N/E115K/E131K,
E90C/E92C/T102A/E115K/E131K,
E90C/E92C/T102V/E115K/E131K,
E90C/E92C/T102N/E115K/E131K,
E90C/E92C/T102Q/E115K/E131K,
E90C/E92C/T104A/E115K/E131K,
E90C/E92C/T104N/E115K/E131K,
E90C/E92C/D105A/E115K/E131K,
E90C/E92C/T102V/T104N/E115K/E131K,
E90C/E92C/T102N/T104N/E115K/E131K,
E90C/E92C/E113K/E115K/E129K/E131K,
E90C/E92P/T100N/E115K/E131K,
E90C/E92P/T102A/E115K/E131K,
E90C/E92P/T102V/E115K/E131K,
E90C/E92P/T102N/E115K/E131K,
E90C/E92P/T102Q/E115K/E131K,
E90C/E92P/T104A/E115K/E131K,
E90C/E92P/T104N/E115K/E131K,
E90C/E92P/D105A/E115K/E131K,
E90C/E92P/T102V/T104N/E115K/E131K,
E90C/E92P/T102N/T104N/E115K/E131K,
E90C/E92P/E113K/E115K/E129K/E131K,
E90P/E92C/T100N/E115K/E131K,
E90P/E92C/T102A/E115K/E131K,
E90P/E92C/T102V/E115K/E131K,
E90P/E92C/T102N/E115K/E131K,
E90P/E92C/T102Q/E115K/E131K,
E90P/E92C/T104A/E115K/E131K,
E90P/E92C/T104N/E115K/E131K,
E90P/E92C/D105A/E115K/E131K,
E90P/E92C/T102V/T104N/E115K/E131K,
E90P/E92C/T102N/T104N/E115K/E131K, or
E90P/E92C/E113K/E115K/E129K/E131K.

The variant preferably comprises or consists of the mutations shown immediately above for SEQ ID NO: 6. The variant may be any of the mutants shown in Table 3 or Table 4.

The variant of SEQ ID NO: 6 is preferably not identical to SEQ ID NO: 2 or 4 (i.e., does not share 100% amino acid identity to SEQ ID NO: 2 or 4).

### Variants

In addition to the specific mutations discussed above, the variant may include other mutations.

Over the entire length of the amino acid sequence of SEQ ID NO: 2, 4 or 6, a variant will preferably be at least 70% homologous to that sequence based on amino acid identity. More preferably, the variant may be at least 75%, at least 80%, at least 85%, at least 90% and more preferably at least 95%, 97%, 98% or 99% homologous based on amino acid identity to the amino acid sequence of SEQ ID NO: 2, 4 or 6 over the entire sequence. Over the entire length of the amino acid sequence of SEQ ID NO: 2, 4 or 6, a variant will preferably be at least 75%, at least 80%, at least 85%, at least 90% and more preferably at least 95%, 97%, 98% or 99% identical to the amino acid sequence of SEQ ID NO: 2, 4 or 6 over the entire sequence.

Reference to homology or identity over an entire sequence may be called “global” homology or identity.

There may be at least 80%, for example at least 85%, 90% or 95%, amino acid identity over a stretch of 100 or more, for example 125, 150, 175 or 200 or more, contiguous amino acids (“hard homology”).

Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/). Amino acid substitutions may be made to the amino acid sequence of SEQ ID NO: 2, 4 or 6 in addition to those discussed above, for example up to 1, 2, 3, 4, 5, 10, 20 or 30 substitutions. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 above. Where amino acids have similar polarity, this can also be determined by reference to the hydropathy scale for amino acid side chains in Table 2.

One or more amino acid residues of the amino acid sequence of SEQ ID NO: 2, 4 or 6 may additionally be deleted from the polypeptides described above. Up to 1, 2, 3, 4, 5, 10, 20 or 30 residues may be deleted, or more.

Variants may include fragments of SEQ ID NO: 2, 4 or 6. Such fragments retain pore forming activity. Fragments may be at least 50, at least 100, at least 150, at least 200 or at least 250 amino acids in length. Such fragments may be used to produce the pores of the invention. A fragment preferably comprises the pore forming domain of SEQ ID NO: 2, 4 or 6. Fragments preferably include amino acids 9 to 200 of SEQ ID NO: 2, 4 or 6.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2, 4 or 6 and which retains its ability to form a pore. A variant typically contains the regions of SEQ ID NO: 2, 4 or 6 that are responsible for pore formation. A variant of SEQ ID NO: 2, 4 or 6 typically comprises the regions in SEQ ID NO: 2, 4 or 6 that form β-strands. One or more modifications can be made to the regions of SEQ ID NO: 2, 4 or 6 that form β-strands as long as the resulting variant retains its ability to form a pore. A variant of SEQ ID NO: 2, 4 or 6 preferably includes one or more modifications, such as substitutions, additions, or deletions, within its a-helices and/or loop regions.

### Constructs

The invention also provides a construct comprising two or more covalently attached monomers derived from Rhodococcus. The construct of the invention retains its ability to form a pore. One or more constructs of the invention may be used to form pores for characterizing target analytes, such as sequencing polypeptides or polynucleotides. The construct may comprise 2, 3, 4, 5, 6, 7, 8, 9 or 10 monomers. The two or more monomers may be the same or different.

The monomers do not have to be mutant monomers of the invention. For instance, all of the monomers may comprise the sequence shown in SEQ ID NO: 2, 4 or 6. For instance, at least one monomer may comprise the sequence shown in SEQ ID NO: 2, 4 or 6. Alternatively, at least one monomer may comprise a variant of SEQ ID NO: 2, 4 or 6 which is at least 50% homologous to SEQ ID NO: 2, 4 or 6 over its entire sequence based on amino acid identity, but does not include any of the specific mutations required by the mutant monomers of the invention. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2, 4 or 6 over the entire sequence. In a preferred embodiment, at least one monomer in the construct is a mutant monomer of the invention. All of the monomers in the construct may be a mutant monomer of the invention. The mutant monomers may be the same or different. In a more preferred embodiment, the construct comprises two monomers and at least one of the monomers is a mutant monomer of the invention.

The monomers are preferably genetically fused. Monomers are genetically fused if the whole construct is expressed from a single polynucleotide sequence. The coding sequences of the monomers may be combined in any way to form a single polynucleotide sequence encoding the construct.

The monomers may be genetically fused in any configuration. The monomers may be fused via their terminal amino acids. For instance, the amino terminus of the one monomer may be fused to the carboxy terminus of another monomer. If the construct is formed from the genetic fusion of two or more monomers each comprising the sequence shown in SEQ ID NO: 2, 4 or 6 or a variant thereof, the second and subsequent monomers in the construct (in the amino to carboxy direction) may comprise a methionine at their amino terminal ends (each of which is fused to the carboxy terminus of the previous monomer). For instance, if M is a monomer comprising the sequence shown in SEQ ID NO: 2, 4 or 6 or a variant (without an amino terminal methionine) and mM is a monomer comprising the sequence shown in SEQ ID NO: 2, 4 or 6 or a variant with an amino terminal methionine, the construct may comprise the sequence M-mM, M-mM-mM or M-mM-mM-mM. The presences of these methionines typically results from the expression of the start codons (i.e., ATGs) at the 5′ end of the polynucleotides encoding the second or subsequent monomers within the polynucleotide encoding entire construct. The first monomer in the construct (in the amino to carboxy direction) may also comprise a methionine (e.g., mM-mM, mM-mM-mM, or mM-mM-mM-mM).

The two or more monomers may be genetically fused directly together. The monomers are preferably genetically fused using a linker. The linker may be designed to constrain the mobility of the monomers. Preferred linkers are amino acid sequences (i.e., peptide linkers). Any of the peptide linkers discussed above may be used.

In another preferred embodiment, the monomers are chemically fused. A subunit is chemically fused to an enzyme if the two parts are chemically attached, for instance via a chemical crosslinker. Any of the chemical crosslinkers discussed above may be used. The linker may be attached to one or more cysteine residues introduced into a mutant monomer of the invention. Alternatively, the linker may be attached to a terminus of one of the monomers in the construct.

If a construct contains different monomers, crosslinkage of monomers to themselves may be prevented by keeping the concentration of linker in a vast excess of the monomers. Alternatively, a “lock and key” arrangement may be used in which two linkers are used. Only one end of each linker may react together to form a longer linker and the other ends of the linker each react with a different monomer. Such linkers are described in International Application No. PCT/GB10/000132 (published as WO 2010/086602).

### Polynucleotides

The present invention also provides polynucleotide sequences which encode a mutant pore of the invention or a mutant monomer of the invention. The mutant pore or the mutant monomer may be any of those discussed above. The polynucleotide sequence preferably comprises a sequence at least 50%, 60%, 70%, 80%, 90% or 95% homologous based on nucleotide identity to the sequence of SEQ ID NO: 1, 3 or 5 over the entire sequence. The polynucleotide sequence preferably comprises a sequence at least 50%, 60%, 70%, 80%, 90% or 95% identical to the sequence of SEQ ID NO: 1, 3 or 5 over the entire sequence. Reference to homology or identity over an entire sequence may be called “global” homology or identity.

There may be at least 80%, for example at least 85%, 90% or 95% nucleotide identity over a stretch of 300 or more, for example 375, 450, 525 or 600 or more, contiguous nucleotides (“hard homology”). Homology may be calculated as described above. The polynucleotide sequence may comprise a sequence that differs from SEQ ID NO: 1, 3 or 5 on the basis of the degeneracy of the genetic code.

The present invention also provides polynucleotide sequences which encode any of the genetically fused constructs of the invention. The polynucleotide preferably comprises two or more sequences as shown in SEQ ID NO: 1, 3 or 5 or a variant thereof as described above.

Polynucleotide sequences may be derived and replicated using standard methods in the art.

Chromosomal DNA encoding wild-type Msp may be extracted from a pore producing organism, such as Rhodococcus corynebacteroides. The gene encoding the pore subunit may be amplified using PCR involving specific primers. The amplified sequence may then undergo site-directed mutagenesis. Suitable methods of site-directed mutagenesis are known in the art and include, for example, combine chain reaction. Polynucleotides encoding a construct of the invention can be made using well-known techniques, such as those described in Sambrook, J. and Russell, D. (2001). Molecular Cloning: A Laboratory Manual, 3rd Edition. Cold Spring Harbor Laboratory Press, Cold Spring Harbor, N.Y.

The resulting polynucleotide sequence may then be incorporated into a recombinant replicable vector such as a cloning vector. The vector may be used to replicate the polynucleotide in a compatible host cell. Thus, polynucleotide sequences may be made by introducing a polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into suitable expression vector. In an expression vector, the polynucleotide sequence is typically operably linked to a control sequence which is capable of providing for the expression of the coding sequence by the host cell. Such expression vectors can be used to express a pore subunit.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide sequences may be introduced into the vector.

The expression vector may then be introduced into a suitable host cell. Thus, a mutant monomer or construct of the invention can be produced by inserting a polynucleotide sequence into an expression vector, introducing the vector into a compatible bacterial host cell, and growing the host cell under conditions which bring about expression of the polynucleotide sequence. The recombinantly-expressed monomer or construct may self-assemble into a pore in the host cell membrane. Alternatively, the recombinant pore produced in this manner may be removed from the host cell and inserted into another membrane. When producing pores comprising at least two different subunits, the different subunits may be expressed separately in different host cells as described above, removed from the host cells, and assembled into a pore in a separate membrane, such as a rabbit cell membrane.

The vectors may be for example, plasmid, virus, or phage vectors provided with an origin of replication, optionally a promoter for the expression of the said polynucleotide sequence and optionally a regulator of the promoter. The vectors may contain one or more selectable marker genes, for example a tetracycline resistance gene. Promoters and other expression regulation signals may be selected to be compatible with the host cell for which the expression vector is designed. A T7, trc, lac, ara or λ_{L} promoter is typically used.

The host cell typically expresses the pore subunit at a high level. Host cells transformed with a polynucleotide sequence will be chosen to be compatible with the expression vector used to transform the cell. The host cell is typically bacterial and preferably Escherichia coli. Any cell with a λ DE3 lysogen, for example C41 (DE3), BL21 (DE3), JM109 (DE3), B834 (DE3), TUNER, Origami and Origami B, can express a vector comprising the T7 promoter. In addition to the conditions listed above any of the methods cited in Proc Natl Acad Sci USA. 2008 Dec. 30; 105(52):20647-52 may be used to express the proteins.

### Pores

The invention also provides various pores, including those described above. The pores of the invention are ideal for characterizing analytes, such as sequencing polypeptides or polynucleotides, because they can discriminate between different amino acids or nucleotides with a high degree of sensitivity. The pores can surprisingly distinguish between the four nucleotides in DNA and RNA. The pores of the invention can even distinguish between methylated and unmethylated nucleotides. The base resolution of pores of the invention is surprisingly high. The pores show almost complete separation of all four DNA nucleotides. The pores further discriminate between deoxycytidine monophosphate (dCMP) and methyl-dCMP based on the dwell time in the pore and the current flowing through the pore.

The pores of the invention can also discriminate between different amino acids or nucleotides under a range of conditions. In particular, the pores will discriminate between amino acids or nucleotides under conditions that are favourable to the characterizing analytes, such as sequencing of polypeptides or polynucleotides. The extent to which the pores of the invention can discriminate between different amino acids or nucleotides can be controlled by altering the applied potential, the salt concentration, the buffer, the temperature, and the presence of additives, such as urea, betaine and DTT. This allows the function of the pores to be fine-tuned, particularly when sequencing. This is discussed in more detail below. The pores of the invention may also be used to identify polypeptides or polynucleotides from the interaction with one or more monomers rather than on an amino acid-by-amino acid basis or nucleotide-by-nucleotide basis.

A pore of the invention may be isolated, substantially isolated, purified, or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as lipids or other pores.

Alternatively, a pore of the invention may be present in a lipid bilayer.

A pore of the invention may be present as an individual or single pore. Alternatively, a pore of the invention may be present in a homologous or heterologous population of two or more pores.

### Homo-Oligomeric Pores

The invention also provides a homo-oligomeric pore comprising identical mutant monomers of the invention. The homo-oligomeric pore of the invention is ideal for characterizing analytes, such as sequencing polypeptides or polynucleotides. The homo-oligomeric pore of the invention may have any of the advantages discussed above.

The homo-oligomeric pore may contain any number of mutant monomers. The pore typically comprises 2, 3, 4, 5, 6, 7, 8, 9 or 10 identical mutant monomers. The pore preferably comprises 7 or 8 identical mutant monomers. One or more, such as 2, 3, 4, 5, 6, 7, 8, 9 or 10, of the mutant monomers is preferably chemically modified as discussed above.

Methods for making pores are discussed in more detail below.

### Hetero-Oligomeric Pores

The invention also provides a hetero-oligomeric pore comprising at least one mutant monomer of the invention, wherein at least one of the monomers differs from the others. The hetero-oligomeric pore of the invention is ideal for characterizing analytes, such as sequencing polypeptides or polynucleotides. Hetero-oligomeric pores can be made using methods known in the art (e.g., Protein Sci. 2002 July; 11(7):1813-24).

The hetero-oligomeric pore contains sufficient monomers to form the pore. The monomers may be of any type. The pore typically comprises 2, 3, 4, 5, 6, 7, 8, 9 or 10 monomers. The pore preferably comprises 7 or 8 monomers.

The pore may comprise at least one monomer comprising (a) the sequence shown in SEQ ID NO: 2, 4 or 6 or (b) a variant thereof which does not have a mutation required by the mutant monomers of the invention. Suitable variants are discussed above. In this embodiment, the remaining monomers are preferably mutant monomers of the invention. Hence, the pore may comprise 9, 8, 7, 6, 5, 4, 3, 2 or 1 mutant monomers of the invention.

In a preferred embodiment, the pore comprises (a) one mutant monomer of the invention and (b) seven identical monomers, wherein the mutant monomer in (a) is different from the identical monomers in (b). The identical monomers in (b) preferably comprise (i) the sequence shown in SEQ ID NO: 2, 4 or 6 or (ii) a variant thereof which does not have a mutation present in the mutant monomers of the invention.

In another preferred embodiment, all of the monomers (i.e., 10, 9, 8, 7, 6, 5, 4, 3 or 2 of the monomers) are mutant monomers of the invention and at least one of them differs from the others. In a more preferred embodiment, the pore comprises eight mutant monomers of the invention and at least one of them differs from the others.

In all the embodiments discussed above, one or more, such as 2, 3, 4, 5, 6, 7, 8, 9 or 10, of the mutant monomers is preferably chemically modified as discussed above. Preferred pores (a) to (c) above are preferably chemically modified by attachment of a molecule to one or more of the introduced cysteines.

Methods for making pores are discussed in more detail below.

### Construct-Containing Pores

The invention also provides a pore comprising at least one construct of the invention. A construct of the invention comprises two or more covalently attached Rhodococcus porin monomers. In other words, a construct must contain more than one monomer. The pore contains sufficient constructs and, if necessary, monomers to form the pore. For instance, an octameric pore may comprise (a) two constructs each comprising four monomers or (b) one construct comprising two monomers and six monomers that do not form part of a construct. At least two of the monomers in the pore are in the form of a construct of the invention. The monomers may be of any type. The pore typically comprises 2, 3, 4, 5, 6, 7, 8, 9 or 10 monomers in total (at least two of which must be in a construct). The pore preferably comprises 7 or 8 monomers (at least two of which must be in a construct).

A pore typically contains (a) one construct comprising two monomers and (b) 1, 2, 3, 4, 5, 6, 7 or 8 monomers. The construct may be any of those discussed above. The monomers may be any of those discussed above, including mutant monomers of the invention.

Another typical pore comprises more than one construct of the invention, such as two, three or four constructs of the invention. Such pores further comprise sufficient monomers to form the pore. The monomer may be any of those discussed above. A further pore of the invention comprises only constructs comprising 2 monomers, for example a pore may comprise 1, 2, 3, 4, 5, 6, 7 or 8 constructs comprising 2 monomers. A specific pore according to the inventions comprises four constructs each comprising two monomers. The constructs may oligomerize into a pore with a structure such that only one monomer of a construct contributes to the barrel or vestibule of the pore. Typically, the other monomers of the construct will be on the outside of the barrel or vestibule of the pore. For example, pores of the invention may comprise 1, 2, 3, 4, 5, 6, 7 or 8 constructs comprising 2 monomers where the barrel or vestibule comprises 8 monomers. Mutations can be introduced into the construct as described above. The mutations may be alternating, i.e., the mutations are different for each monomer within a two-monomer construct and the constructs are assembled as a homo-oligomer resulting in alternating modifications. In other words, monomers comprising MutA and MutB are fused and assembled to form an A-B:A-B:A-B:A-B pore. Alternatively, the mutations may be neighbouring, i.e., identical mutations are introduced into two monomers in a construct, and this is then oligomerized with different mutant monomers. In other words, monomers comprising MutA are fused follow by oligomerisation with MutB-containing monomers to form A-A:B:B:B:B:B:B.

One or more of the monomers of the invention in a construct-containing pore may be chemically modified as discussed below.

### Method for Making Modified Proteins

Methods for introducing or substituting non-naturally occurring amino acids are also well known in the art. For instance, non-naturally occurring amino acids may be introduced by including synthetic aminoacyl-tRNAs in the IVTT system used to express the mutant monomer.

Alternatively, they may be introduced by expressing the mutant monomer in E. coli that are auxotrophic for specific amino acids in the presence of synthetic (i.e., non-naturally occurring) analogues of those specific amino acids. They may also be produced by naked ligation if the mutant monomer is produced using partial peptide synthesis.

Any proteins described herein, especially the mutant monomers of the invention, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the monomer, or subunit, does not naturally contain such a sequence. An alternative to introducing a genetic tag is to chemically react a tag onto a native or engineered position on the protein. An example of this would be to react a gel-shift reagent to a cysteine engineered on the outside of the protein.

The protein or monomer may be labelled with a revealing label. The revealing label may be any suitable label which allows the monomer, or subunit, to be detected. Suitable labels include, but are not limited to, fluorescent molecules, radioisotopes, e.g., ¹²⁵I, ³⁵S, enzymes, antibodies, antigens, polynucleotides, and ligands such as biotin.

The protein or monomer may, in one embodiment, be produced using D-amino acids. For instance, the protein or monomer may comprise a mixture of L-amino acids and D-amino acids.

This is conventional in the art for producing such proteins or peptides.

The protein or monomer may comprise one or more specific modifications to facilitate amino acid or nucleotide discrimination. The protein or monomer may also contain other non-specific modifications as long as they do not interfere with pore formation. A number of non-specific side chain modifications are known in the art and may be made to the side chains of amino acids in the transmembrane protein nanopore and/or auxiliary protein. Such modifications include, for example, reductive alkylation of amino acids by reaction with an aldehyde followed by reduction with NaBH₄, amidination with methylacetimidate or acylation with acetic anhydride.

The protein or monomer can be produced using standard methods known in the art. The protein or monomer may be made synthetically or by recombinant means. For example, the proteins may be synthesised by in vitro translation and transcription (IVTT). The amino acid sequence of the protein may be modified to include non-naturally occurring amino acids or to increase the stability of the protein. When a protein is produced by synthetic means, such amino acids may be introduced during production. The protein may also be altered following either synthetic or recombinant production. Suitable methods for producing transmembrane protein nanopores are discussed in International applications WO 2010/004273, WO 2010/004265, or WO 2010/086603. Methods for inserting pores into membranes are known.

Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression.

Typical protein liquid chromatography systems include FPLC, AKTA systems, the Bio-Cad system, the Bio-Rad BioLogic system, and the Gilson HPLC system.

Two or more monomers may be covalently attached to one another. For example, at least 2, at least 3, at least 4, at least 5, at least 6, at least 7, at least 8, at least 9 or at least 10 monomers, or subunits, may be covalently attached. The covalently attached monomers, or subunits, may be the same or different.

The monomers, or subunits, may be genetically fused, optionally via a linker, or chemically fused, for instance via a chemical crosslinker. Methods for covalently attaching monomers, or subunits, are disclosed in WO2017/149316, WO2017/149317 and WO2017/149318.

### Chemical Modification

In some embodiments, the mutant monomer is chemically modified. The mutant monomer can be chemically modified in any way and at any site. The mutant monomer may, for example, be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant monomer may be chemically modified by the attachment of any molecule. For instance, the mutant monomer may be chemically modified by attachment of a dye or a fluorophore.

In some embodiments, the mutant monomer is chemically modified with a molecular adaptor that facilitates the interaction between a pore comprising the monomer and a target analyte, such as a target amino acid, target polypeptide, target nucleotide or target polynucleotide sequence.

The presence of the adaptor improves the host-guest chemistry of the pore and the target analyte and thereby improves the sequencing ability of pores formed from the mutant monomer. The principles of host-guest chemistry are well-known in the art. The adaptor has an effect on the physical or chemical properties of the pore that improves its interaction with the target analyte, such as the target amino acid, polypeptide, nucleotide, or polynucleotide. The adaptor may alter the charge of the barrel or channel of the pore or specifically interact with or bind to the target analyte thereby facilitating its interaction with the pore.

The molecular adaptor is preferably a cyclic molecule, a cyclodextrin, a species that is capable of hybridization, a DNA binder or interchelator, a peptide or peptide analogue, a synthetic polymer, an aromatic planar molecule, a small positively charged molecule or a small molecule capable of hydrogen-bonding.

The adaptor may be cyclic. A cyclic adaptor preferably has the same symmetry as the pore. The adaptor preferably has two-fold, three-fold, four-fold, five-fold, six-fold, seven-fold, eight-fold, nine-fold, or ten-fold symmetry since the pores typically have 2 to 10 subunits around a central axis. This is discussed in more detail above.

The adaptor typically interacts with the target analyte, such as amino acid, polypeptide, nucleotide, or polynucleotide, via host-guest chemistry. The adaptor is typically capable of interacting with the target analyte. The adaptor comprises one or more chemical groups that are capable of interacting with the target analyte. The one or more chemical groups preferably interact with the target analyte by non-covalent interactions, such as hydrophobic interactions, hydrogen bonding, Van der Waal's forces, n-cation interactions and/or electrostatic forces. The one or more chemical groups that are capable of interacting with the target analyte are preferably positively charged. The one or more chemical groups that are capable of interacting with the target analyte more preferably comprise amino groups. The amino groups can be attached to primary, secondary, or tertiary carbon atoms. The adaptor even more preferably comprises a ring of amino groups, such as a ring of 6, 7 or 8 amino groups. The adaptor most preferably comprises a ring of eight amino groups. A ring of protonated amino groups may interact with negatively charged phosphate groups in the target analyte.

The correct positioning of the adaptor within the pore can be facilitated by host-guest chemistry between the adaptor and the pore comprising the mutant monomer. The adaptor preferably comprises one or more chemical groups that are capable of interacting with one or more amino acids in the pore. The adaptor more preferably comprises one or more chemical groups that are capable of interacting with one or more amino acids in the pore via non-covalent interactions, such as hydrophobic interactions, hydrogen bonding, Van der Waal's forces, n-cation interactions and/or electrostatic forces. The chemical groups that are capable of interacting with one or more amino acids in the pore are typically hydroxyls or amines. The hydroxyl groups can be attached to primary, secondary, or tertiary carbon atoms. The hydroxyl groups may form hydrogen bonds with uncharged amino acids in the pore. Any adaptor that facilitates the interaction between the pore and the target analyte, such as amino acid, polypeptide, nucleotide, or polynucleotide, can be used.

Suitable adaptors include, but are not limited to, cyclodextrins, cyclic peptides and cucurbiturils. The adaptor is preferably a cyclodextrin or a derivative thereof. The cyclodextrin or derivative thereof may be any of those disclosed in Eliseev, A. V., and Schneider, H-J. (1994) J. Am. Chem. Soc. 116, 6081-6088. The adaptor is more preferably heptakis-6-amino-β-cyclodextrin (am7-βCD), 6-monodeoxy-6-monoamino-β-cyclodextrin (am1-βCD) or heptakis-(6-deoxy-6-guanidino)-cyclodextrin (gu7-βCD). The guanidino group in gu7-βCD has a much higher pKa than the primary amines in am7-βCD and so it is more positively charged. This gu7-βCD adaptor may be used to increase the dwell time of the amino acid or nucleotide in the pore, to increase the accuracy of the residual current measured, as well as to increase the base detection rate at high temperatures or low data acquisition rates.

If a succinimidyl 3-(2-pyridyldithio)propionate (SPDP) crosslinker is used as discussed in more detail below, the adaptor is preferably heptakis(6-deoxy-6-amino)-6-N-mono(2-pyridyl)dithiopropanoyl-β-cyclodextrin (am6amPDP1-βCD).

More suitable adaptors include γ-cyclodextrins, which comprise 2, 3, 4, 5, 6, 7, 8, 9 or 10 sugar units. The γ-cyclodextrin may contain a linker molecule or may be modified to comprise all or more of the modified sugar units used in the β-cyclodextrin examples discussed above.

The molecular adaptor may be covalently attached to the mutant monomer. The adaptor can be covalently attached to the pore using any method known in the art. The adaptor is typically attached via chemical linkage. If the molecular adaptor is attached via cysteine linkage, the one or more cysteines have preferably been introduced to the mutant, for instance in the barrel, by substitution. The mutant monomer may be chemically modified by attachment of a molecular adaptor to one or more cysteines introduced into the mutant monomer, for instance by substitution.

The reactivity of cysteine residues may be enhanced by modification of the adjacent residues.

For instance, the basic groups of flanking arginine, histidine or lysine residues will change the pKa of the cysteines thiol group to that of the more reactive S— group. The reactivity of cysteine residues may be protected by thiol protective groups such as dTNB. These may be reacted with one or more cysteine residues of the mutant monomer before a linker is attached.

The molecule may be attached directly to the mutant monomer. The molecule is preferably attached to the mutant monomer using a linker, such as a chemical crosslinker or a peptide linker.

Suitable chemical crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octanoate.

The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule. Suitable examples of peptide linkers are defined above.

The linker is preferably resistant to dithiothreitol (DTT). Suitable linkers include, but are not limited to, iodoacetamide-based and Maleimide-based linkers.

In other embodiment, the mutant monomer may be attached to a polynucleotide binding protein.

This forms a modular sequencing system that may be used in the methods of sequencing of the invention. The polynucleotide binding protein may be a nucleic acid handling enzyme, for example a translocase, a polymerase, or a helicase.

The polynucleotide binding protein is preferably covalently attached to the mutant monomer.

The protein can be covalently attached to the monomer using any method known in the art. The monomer and protein may be chemically fused or genetically fused. The monomer and protein are genetically fused if the whole construct is expressed from a single polynucleotide sequence.

Genetic fusion of a monomer to a polynucleotide binding protein is discussed in WO 2010/004265.

If the polynucleotide binding protein is attached via cysteine linkage, the one or more cysteines have preferably been introduced to the mutant by substitution. The one or more cysteines are preferably introduced into loop regions which have low conservation amongst homologues indicating that mutations or insertions may be tolerated. They are therefore suitable for attaching a polynucleotide binding protein. The reactivity of cysteine residues may be enhanced by modification as described above.

The polynucleotide binding protein may be attached directly to the mutant monomer or via one or more linkers. The molecule may be attached to the mutant monomer using the hybridization linkers described in as WO 2010/086602. Alternatively, peptide linkers may be used. Peptide linkers are amino acid sequences. The length, flexibility and hydrophilicity of the peptide linker are typically designed such that it does not to disturb the functions of the monomer and molecule. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

The mutant monomer may be chemically modified with a molecular adaptor and a polynucleotide binding protein.

### Method of Producing Pores

The invention also provides methods to produce the pores of the invention in vivo and in vitro.

One embodiment provides a method for producing a pore of the invention via co-expression. The method comprises expressing the necessary monomers in a suitable host cell and allowing in vivo pore formation.

Another method for producing a pore of the invention relates to in vitro reconstitution of said monomers to obtain a functional pore. Said method comprises the steps of contacting the necessary monomers in a suitable system to allow complex formation. Said system may be an “in vitro system”, which refers to a system comprising at least the necessary components and environment to execute said method, and makes use of biological molecules, organisms, a cell (or part of a cell) outside of their normal naturally occurring environment, permitting a more detailed, more convenient, or more efficient analysis than can be done with whole organisms. An in vitro system may also comprise a suitable buffer composition provided in a test tube, wherein said protein components to form the complex have been added. A person skilled in the art is aware of the options to provide said system.

One some or all of the monomers may be tagged to facilitate purification. Purification can also be performed when the monomers are untagged. Methods known in the art (e.g., ion exchange, gel filtration, hydrophobic interaction column chromatography etc.) can be used alone or in different combinations to purify the components of the pore.

### Kits

The invention also provides a kit for characterising a target polynucleotide or a target polypeptide. The kit comprises a Rhodococcus pore, preferably an isolated Rhodococcus pore, and a polynucleotide binding protein or a polypeptide handling enzyme.

The Rhodococcus pore may be a wild-type pore. The pore may be formed from a monomer comprising the sequence shown in SEQ ID NO; 2, 4 or 6. The pore may comprise 2, 3, 4, 5, 6, 7, 8, 9 or 10 monomers comprising the sequence shown in SEQ ID NO; 2, 4 or 6, preferably 2, 3, 4, 5, 6, 7, 8, 9 or 10 identical monomers. The pore is preferably a pore of the invention and may be any of the pores described above, including mutant pores, hetero-oligomeric pores, homo-oligomeric pores, and construct-containing pores.

The polynucleotide binding protein may be any of those discussed above. The polynucleotide binding protein or polypeptide handling enzyme may be attached to the pore as discussed above.

The kit preferably further comprises the components of a membrane. The kit may comprise components of any type of membranes, such as an amphiphilic layer or a triblock copolymer membrane. The kit may further comprise one or more anchors, such as cholesterol, for coupling the target polynucleotide to the membrane. The kit may further comprise one or more polynucleotide adaptors that can be attached to a target polynucleotide to facilitate characterisation of the polynucleotide. In one embodiment, the anchor, such as cholesterol, is attached to the polynucleotide adaptor.

The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in analyte characterization.

### Apparatus

The invention also provides an apparatus for characterising a target polynucleotide or a target polypeptide in a sample. The apparatus comprises a plurality of Rhodococcus pores, preferably a plurality of isolated Rhodococcus pores, and a plurality of polynucleotide binding proteins or a plurality of polypeptide handling enzymes. The Rhodococcus pores may be any of those discussed above with reference to the kits of the invention, including wild-type pores. The Rhodococcus pores are preferably pores of the invention and may be any of the pores described above, including mutant pores, hetero-oligomeric pores, homo-oligomeric pores, and construct-containing pores. The polynucleotide binding proteins may be any of those discussed above. The polynucleotide binding proteins or polypeptide handling enzymes may be attached to the pores as discussed above.

The invention also provides an apparatus comprising a transmembrane protein pore inserted into an in vitro membrane, wherein the transmembrane protein pore comprises at least one monomer which is a variant of the Rhodococcus porin monomer and comprising mutations at one or more of the positions in (a), (b) and/or (c), corresponding to the sequence shown in SEQ ID NO: 2.

The positions in (a), (b) and/or (c) with regards to SEQ ID NO: 2 are discussed above with reference to the pores and monomers of the invention.

The invention also provides an apparatus produced by a method comprising: (i) obtaining a transmembrane protein pore comprising at least one Rhodococcus porin monomer, wherein the monomer comprises amino acid mutations at one or more of positions in (a), (b) and/or (c) corresponding to the sequence shown in SEQ ID NO: 2; and (ii) contacting the pore with an in vitro membrane such that the pore is inserted in the in vitro membrane. The positions in (a), (b) and/or (c) with regards to SEQ ID NO: 2 are discussed above with reference to the pores and monomers of the invention.

The invention also provides an apparatus comprising a transmembrane protein pore inserted into an in vitro membrane, wherein the transmembrane protein pore comprises at least one monomer which is a variant of the Rhodococcus porin monomer and comprising mutations at one or more of the positions in (a), (b) and/or (c), corresponding to the sequence shown in SEQ ID NO: 4.

The positions in (a), (b) and/or (c) with regards to SEQ ID NO: 4 are discussed above with reference to the pores and monomers of the invention.

The invention also provides an apparatus produced by a method comprising: (i) obtaining a transmembrane protein pore comprising at least one Rhodococcus porin monomer, wherein the monomer comprises amino acid mutations at one or more of positions in (a), (b) and/or (c) corresponding to the sequence shown in SEQ ID NO: 4; and (ii) contacting the pore with an in vitro membrane such that the pore is inserted in the in vitro membrane. The positions in (a), (b) and/or (c) with regards to SEQ ID NO: 4 are discussed above with reference to the pores and monomers of the invention.

The invention also provides an apparatus comprising a transmembrane protein pore inserted into an in vitro membrane, wherein the transmembrane protein pore comprises at least one monomer which is a variant of the Rhodococcus porin monomer and comprising mutations at one or more of the positions in (a), (b) and/or (c), corresponding to the sequence shown in SEQ ID NO: 6.

The positions in (a), (b) and/or (c) with regards to SEQ ID NO: 6 are discussed above with reference to the pores and monomers of the invention.

The invention also provides an apparatus produced by a method comprising: (i) obtaining a transmembrane protein pore comprising at least one Rhodococcus porin monomer, wherein the monomer comprises amino acid mutations at one or more of positions in (a), (b) and/or (c) corresponding to the sequence shown in SEQ ID NO: 6; and (ii) contacting the pore with an in vitro membrane such that the pore is inserted in the in vitro membrane. The positions in (a), (b) and/or (c) with regards to SEQ ID NO: 6 are discussed above with reference to the pores and monomers of the invention.

Any of the specific embodiments discussed above, especially in relation to the pores and monomers of the invention, are equally applicable to the apparatuses of the invention.

### Membrane Comprising Pores

The invention also provides a membrane comprising a Rhodococcus pore, preferably an isolated Rhodococcus pore. The pore is preferably present in the membrane, together forming a transmembrane pore. The membrane may comprise components of any type of membranes, such as an amphiphilic layer or a triblock copolymer membrane. The membrane may further comprise a polynucleotide binding protein or a polypeptide handling enzyme attached to the pore. The membrane may further comprise one or more anchors for coupling the polynucleotide or polypeptide to the membrane. The Rhodococcus pore may be any of those discussed above with reference to the kits of the invention, including wild-type pores. The Rhodococcus pore is preferably a pore of the invention and may be any of the pores described above, including mutant pores, hetero-oligomeric pores, homo-oligomeric pores, and construct-containing pores.

### Arrays

The invention also provides an array comprising a plurality of membranes of the invention. Any of the embodiments discussed above with respect to the membrane of the invention equally apply the array of the invention. The array may be set up to perform any of the methods described below.

In a preferred embodiment, each membrane in the array comprises one pore. Due to the manner in which the array is formed, for example, the array may comprise one or more membranes that do not comprise a pore, and/or one or more membranes that comprise two or more pores. The array may comprise from about 2 to about 1000, such as from about 10 to about 800, from about 20 to about 600 or from about 30 to about 500 membranes.

### System

The invention provides a system comprising (a) a membrane of the invention or an array of the invention, (b) means for applying a potential across the membrane(s) and (c) means for detecting electrical or optical signals across the membrane(s).

The invention also provides a system comprising (a) a membrane or a plurality of membranes comprising a Rhodococcus pore, (b) means for applying a potential across the membrane(s) and (c) means for detecting electrical or optical signals across the membrane(s). The Rhodococcus pore may be any of those discussed above with reference to the kits of the invention, including wild-type pores. The Rhodococcus pore is preferably a pore of the invention and may be any of the pores described above, including mutant pores, hetero-oligomeric pores, homo-oligomeric pores, and construct-containing pores.

The pores and membranes may be any as described herein above.

In one embodiment, the system further comprises a first chamber and a second chamber, wherein the first and second chambers are separated by the membrane(s). When used to characterise a target analyte, the system may further comprise a target analyte, wherein the target analyte is transiently located within the continuous channel and wherein one end of the target analyte is located in the first chamber and one end of the target analyte is located in the second chamber.

The target analyte is preferably a target polypeptide or a target polynucleotide.

In one embodiment, the system further comprises an electrically conductive solution in contact with the pore(s), electrodes providing a voltage potential across the membrane(s), and a measurement system for measuring the current through the pore(s). In one embodiment, the voltage applied across the membranes and pore is from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different amino acids or nucleotides by a pore by using an increased applied potential. Any suitable electrically conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber.

Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g., in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of an amino acid or nucleotide to be identified against the background of normal current fluctuations.

A buffer may be present in the electrically conductive solution. Typically, the buffer is phosphate buffer. Other suitable buffers are HEPES and Tris-HCl buffer. The pH of the electrically conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane(s) containing the pore(s) are formed. Alternatively, the barrier forms the membrane in which the pore is present.

The apparatus may also comprise an electrical circuit capable of applying a potential and measuring an electrical signal across the membrane and pore.

The apparatus may be any of those described in WO 2008/102120, WO 2009/077734, WO 2010/122293, WO 2011/067559, or WO 00/28312.

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example, a hydrophobic polymer may be made from siloxane or other non-hydrocarbon-based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer-based membranes for a wide range of applications.

The membrane is most preferably one of the membranes disclosed in International Application No. WO2014/064443 or WO2014/064444.

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary.

The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically modified. The head group or the tail group of the lipids may be chemically modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647. If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method is typically carried out using (i) an artificial amphiphilic layer comprising a pore, (ii) an isolated, naturally occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Methods of Characterising an Analyte

The invention also provides a method of determining the presence, absence or one or more characteristics of a target analyte. The method comprises contacting the target analyte with a Rhodococcus pore, such that the target analyte moves with respect to, such as into or through, the pore, respectively, and taking one or more measurements as the analyte moves with respect to the pore and thereby determining the presence, absence or one or more characteristics of the analyte. The Rhodococcus pore may be any of those discussed above with reference to the kits of the invention, including wild-type pores. The Rhodococcus pore is preferably a pore of the invention and may be any of the pores described above, including mutant pores, hetero-oligomeric pores, homo-oligomeric pores, and construct-containing pores. The analyte may pass through the pore constriction. The pore may be present in a membrane.

In one embodiment, the method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore, or in the vicinity of either opening of the channel will have an effect on the open-channel ion flow through the pore, which is the essence of “molecular sensing” of pore channels. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

The target analyte may be a metal ion, an inorganic salt, a polymer, an amino acid, a peptide, a polypeptide, a protein, a nucleotide, an oligonucleotide, a polynucleotide, a polysaccharide, a dye, a bleach, a pharmaceutical, a diagnostic agent, a recreational drug, an explosive, a toxic compound, or an environmental pollutant. The target analyte preferably is or comprises a polypeptide, a polynucleotide, or a polysaccharide. The target analyte may be a metabolite.

The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

In one embodiment, the analyte is an amino acid, a peptide, a polypeptide, or protein. The amino acid, peptide, polypeptide, or protein can be naturally occurring or non-naturally occurring. The polypeptide or protein can include within them synthetic or modified amino acids. Several different types of modification to amino acids are known in the art. Suitable amino acids and modifications thereof are above. It is to be understood that the target analyte can be modified by any method available in the art. The method preferably comprises (i) contacting the polypeptide with a polypeptide handling enzyme capable of controlling the movement of the polypeptide with respect to the pore; and (ii) taking one or more measurements characteristic of the polypeptide as the polypeptide moves with respect to the pore.

In a preferred embodiment, the analyte is a polynucleotide, such as a nucleic acid. The method preferably comprises (i) contacting the polynucleotide with a polynucleotide binding protein capable of controlling the movement of the polynucleotide with respect to the pore; and (ii) taking one or more measurements characteristic of the polynucleotide as the polynucleotide moves with respect to the pore. A polynucleotide is defined as a macromolecule comprising two or more nucleotides. The naturally occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are described in, for example, WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide.

It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction (or “reading head”). It will be appreciated that sequencing may be performed upon an intact nucleic acid polymer that is ‘threaded’ through the pore via the action of an associated polymerase or helicase, for example. Alternatively, sequences may be determined by passage of nucleotide triphosphate bases that have been sequentially removed from a target nucleic acid in proximity to the pore (see for example WO 2014/187924).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate, or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e., lack a nucleobase). A nucleotide may also lack a nucleobase and a sugar (i.e., is a C3 spacer).

The sequence of the nucleotides (iii) is determined by the consecutive identity of following nucleotides attached to each other throughout the polynucleotide strain, in the 5′ to 3′ direction of the strand.

The pore described herein are particularly useful in analysing homopolymers. For example, the pores may be used to determine the sequence of a polynucleotide comprising two or more, such as at least 3, 4, 5, 6, 7, 8, 9 or 10, consecutive nucleotides that are identical. For example, the pores may be used to sequence a polynucleotide comprising a polyA, polyT, polyG and/or polyC region.

In a preferred embodiment, the target analyte comprises a polynucleotide-polypeptide conjugate and said method comprises (i) contacting the conjugate with a polynucleotide binding protein capable of controlling the movement of the polynucleotide of the conjugate with respect to the pore; and (ii) taking one or more measurements characteristic of the polypeptide as the conjugate moves with respect to the pore.

It is to be understood that although particular embodiments, specific configurations as well as materials and/or molecules, have been discussed herein for engineered cells and methods according to the present invention, various changes or modifications in form and detail may be made without departing from the scope and spirit of this invention. The following examples are provided to better illustrate particular embodiments, and they should not be considered limiting the application. The application is limited only by the claims.

The following Examples illustrates the invention.

### EXAMPLES

### Example 1

### Methods

E. coli Pore Production

DNA encoding the mature forms of the PorARr, PorBRr and PorARc proteins was synthesized by GENEWIZ Germany GmbH and Twist Bioscience plc. USA and cloned into a pT7 vector containing ampicillin resistance gene. DNA concentration was adjusted to 400 ng/μL.

The plasmid DNA was thawed at room temperature and mixed by slowly pipetting up and down. Chemically competent Lemo21(DE3) E. coli cells were thawed on ice. 1 μl of DNA at 400 ng/μl was added to the cells and mixed by flicking and agitating the tube. This was then left on ice for 30 minutes before heat shocking the cells at 42° C. for 10 seconds. The cells were then left on ice for 5 minutes. 250 μl of SOC (Sigma, S1797) media that was pre-warmed to 37° C. was added to the cells and left for one hour at 37° C. with shaking at 250 rpm. 75 μl of outgrown cell culture was then plated out on a big LB agar plate containing 100 μg/ml ampicillin and 41 μg/ml chloramphenicol and then left to incubate overnight at 37° C.

A single colony of the transformed Lemo21 (DE3) cells was picked and inoculated in 100 ml LB medium with 100 μg/ml ampicillin and 41 μg/ml chloramphenicol. This starter culture was incubated overnight at 37° C. and 250 rpm in a 500 ml flask. Main culture of 500 ml LB medium containing 100 μg/ml ampicillin and 41 μg/ml chloramphenicol were set up in 2.5 l flask. This was then added spiked with 4 ml of starter culture (dilution 1:125) and the cells were left to divide at 37° C. and 250 rpm until O.D 0.6 was reached. Upon reaching O.D. 0.6, the temperature of the incubator was reduced to 18° C., and the cells were induced with 0.2 mM IPTG (final concentration in the medium). The cells were incubated overnight at 18° C. and 250 rpm to allow overexpression of the protein of interest. Finally, the cells were harvested by spinning them at 6000 g for 20 min at 4° C.

The cell paste was weighed to calculate the right volume of functional lysis buffer to prepare (cells are to be resuspended in 100 ml lysis buffer per 10 g of paste). The required amount of functional lysis buffer was prepared by adding benzonase (10 μl/100 ml, Sigma), tablets of EDTA-free protease inhibitor cocktail (1 tablet/100 ml, Sigma) and 10× BugBuster Protein Extraction Reagent (10 ml/100 ml, Sigma) to buffer containing 50 mM Tris/HCl, 150 mM NaCl, 2 mM EDTA, 0.1% DDM. pH of resulting functional lysis buffer was then adjusted to pH 8 at room temperature. The cells were resuspended in functional lysis buffer and mixed for 1 hour at room temperature with a magnetic stirrer to result in homogenous mixture. The cells were then lysed by sonication on ice (Soniprep150, 10 cycles 10s on/20s off, 10 μm amplitude) and left to solubilize for 3.5h stirring at RT. The cell extract was then transferred to 100 ml Beckman tubes and centrifuged at 40,000 g for 35 minutes at room temperature. Resulting supernatant was then filtered using sterile 0.2 μm vacuum filter (Nalgene Rapid-Flow, Sigma)

### Affinity Chromatography

Filtered supernatant was loaded onto a 5 mL StrepTrap HP column (Fisher, 10571680). The column was washed with 20 column volumes (CV) of Mobile phase A (25 mM Tris, 150 mM NaCl, 2 mM EDTA 0.1% DDM, pH 8.0). The column was then washed with 7 CV of 25 mM Tris, 2 M NaCl, 2 mM EDTA, 0.1% DDM, pH 8.0 buffer before being returned back to Mobile phase A. Elution was carried out by flowing 7 CV of Mobile phase B buffer (25 mM Tris, 150 mM NaCl, 2 mM EDTA 0.1% DDM, 10 mM Desthiobiotin pH 8.0) through the column.

### Anion Exchange Chromatography

Resulting fractions containing Affinity chromatography eluate were pooled together and diluted 5× with AEX Mobile Phase A (25 mM Tris, 0.1% DDM, pH 9.0) to reduce the salt concentration in the sample. Resulting Strep purified sample was then subsequently loaded onto POROS HQ10 2 ml column (ThermoFisher). The column was then washed with 20 CV of AEX Mobile phase A before elution with 2M NaCl over a gradient of 0-50% over 35 CV, where AEX Mobile phase B comprised of 25 mM Tris, 2M NaCl, 0.1% DDM, pH 9.0.

Fractions of interest were screened using non-denaturing Native-PAGE. The fractions that contained the protein of interest in the right oligomeric state were pooled together and heated to 90° C. for 15 min to denature misfolded proteins. Heated sample was then centrifuged at 21,130 g for 10 minutes at RT to pellet denatured proteins. The supernatant was retained and used in electrophysiological recordings.

### Electrophysiology Data

Electrical measurements were acquired from PorARr, PorBRr and PorARc wild-type and mutant nanopores that were inserted into MinION flow cells. After a single pore inserted into the block co-polymer membrane, 1 mL of a buffer comprising 25 mM Potassium Phosphate, 150 mM Potassium Ferrocyanide (II), 150 mM Potassium Ferricyanide (III), pH 8.0 was flowed through the system to remove any excess protein nanopores.

The analyte being used to assess the DNA squiggle was a 3.6-kilobase ssDNA section from the 3′ end of the lambda genome. Preparation of the analyte, ligating the analyte to the Y-adapter, SPRI-bead clean-up of the ligated analyte and addition to a MinION flow cell was carried out using the Oxford Nanopore Technologies Q-SQK-LSK109 protocol.

Electrical measurements were acquired using MinION Mk1b from Oxford Nanopore Technologies. A standard sequencing script at −180 mV was run for 1-6 hours, with static flicks (voltage reversal to +100 mV) every 5 minutes to remove extended nanopore blocks. Raw data was collected in a bulk FAST5 file using MinKNOW software (Oxford Nanopore Technologies).

### Results

Wild-type PorARr, PorBRr and PorARc pores were tested. The results were as follows.

### PorARr

Few pores observed
The few pores that were observed did appear stable
No DNA capture

### PorBRr

A few more pores observed than PorARr but there still were not many
Pores gate a lot
No DNA capture

### PorARc

Lots of pores observed
Pores have some gating
Two pore populations observed
No DNA capture

The results obtained from pores containing mutated PorARr, PorBRr and PorARc monomers are shown in Table 3.

| | | WT | | | |
|---|---|---|---|---|---|
| | | SEQ | | | |
| | | ID | | | |
| Name | Source | NO# | Mutations | Effect | FIG. |
| ONLP1 | PorARc | 2 | — (WT) | | 1 |
| 8153 | | | | | |
| ONLP1 | PorARc | 2 | E78S/D82S/E89N | Increased open pore current | 1 |
| 8300 | | | | (compared to ONLP18153). DNA | |
| | | | | squiggle around open pore level. | |
| | | | | High noise. | |
| ONLP1 | PorARc | 2 | E78S/D82S/E89N/ | Increased open pore current. DNA | 1 |
| 8342 | | | E116T/E125A/ | squiggle around open pore level. | |
| | | | D165S | Increased DNA capture rate. | |
| | | | | Compared to ONLP18300. | |
| ONLP1 | PorARc | 2 | E78S/D82S/E89R | Increased open pore current. | 2 |
| 9696 | | | | Reduced open pore noise. DNA | |
| | | | | squiggle well below open pore | |
| | | | | level, facilitating splitting the | |
| | | | | signal into individual squiggles. | |
| | | | | Increased DNA translocation | |
| | | | | speed. | |
| | | | | Compared to ONLP18300. | |
| ONLP1 | PorARc | 2 | E78R/D82S/E89R | Increased open pore current. DNA | 2 |
| 9697 | | | | squiggle well below open pore | |
| | | | | level. Increased SNR, with a | |
| | | | | reduction in range and median sd. | |
| | | | | Reduced DNA translocation | |
| | | | | speed. | |
| | | | | Compared to ONLP19696. | |
| ONLP1 | PorARc | 2 | E78R/D82S/E89R/ | Increased open pore current. DNA | 2 |
| 8393 | | | E116T/E125A/ | squiggle well below open pore | |
| | | | D165S | level. Increased SNR with a | |
| | | | | reduction in range and median sd. | |
| | | | | Compared to ONLP19697. | |
| ONLP1 | PorARc | 2 | E57R/E78R/D82S/ | DNA squiggle well below open | 3 |
| 9077 | | | E89R/E116T/ | pore level. Reduction in median | |
| | | | E125A/D165S | current of the squiggle and open | |
| | | | | pore level. | |
| | | | | Compared to ONLP18393. | |
| ONLP1 | PorARc | 2 | D55S/E57S/D58S/ | Small increase in open pore level | 3 |
| 9076 | | | E78R/D82S/E89R/ | and median current of the DNA | |
| | | | E116T/E125A/ | squiggle. Increased SNR due to a | |
| | | | D165S | slight increase in range and a | |
| | | | | reduction in median sd. | |
| | | | | Compared to ONLP18393. | |
| ONLP1 | PorARc | 2 | E78R/D82S/E89R/ | Slightly higher open pore level. | 3 |
| 9078 | | | D104S/E116T/E122S/ | Reduction in SNR with a large | |
| | | | E125A/D165S | increase in range and median sd. | |
| | | | | Compared to ONLP18393. | |
| ONLP1 | PorARc | 2 | D55S/E57S/D58S/ | Increase in open pore level and | 4 |
| 9079 | | | E78R/D82S/E89R/ | median current of the DNA | |
| | | | D104S/E116T/E122S/ | squiggle. Reduction in SNR with | |
| | | | E125A/D165S | an increased range and median sd. | |
| | | | | Compared to ONLP19078. | |
| 90NLP | PorARc | 2 | E57R/E78R/D82S/ | Reduced open pore level. | 4 |
| 19080 | | | E89R/D104S/E116T/ | Increased median current. | |
| | | | E122S/E125A/ | Increased SNR due to a reduction | |
| | | | D165S | in median sd. | |
| | | | | Compared to ONLP19078. | |
| ONLP1 | PorARc | 2 | E78R/D82S/E89G/ | DNA squiggle around open pore | 5 |
| 9320 | | | E116T/E125A/ | level. Increased median current. | |
| | | | D165S | Increased SNR due to increase in | |
| | | | | range. | |
| | | | | Compared to ONLP18393. | |
| ONLP1 | PorARc | 2 | E78R/D82S/E89A/ | DNA squiggle around open pore | 5 |
| 9322 | | | E116T/E125A/ | level. Increased median current. | |
| | | | D165S | Increased SNR due to increase in | |
| | | | | range. | |
| | | | | Compared to ONLP18393. | |
| ONLP1 | PorARc | 2 | E78R/D82S/E89V/ | DNA squiggle around open pore | 5 |
| 9325 | | | E116T/E125A/ | level. Increased median current. | |
| | | | D165S | Increased SNR due to increase in | |
| | | | | range. | |
| | | | | Compared to ONLP18393. | |
| ONLP1 | PorARc | 2 | E78R/D82S/E89L/ | DNA squiggle around open pore | 6 |
| 9323 | | | E116T/E125A/ | level. Increased median current. | |
| | | | D165S | Increased SNR due to increase in | |
| | | | | range. | |
| | | | | Compared to ONLP18393. | |
| ONLP1 | PorARc | 2 | E78R/D82S/E89I/ | DNA squiggle around open pore | 6 |
| 9321 | | | E116T/E125A/ | level. Increased median current. | |
| | | | D165S | Small increase in SNR due to | |
| | | | | increase in range. | |
| | | | | Compared to ONLP18393. | |
| ONLP1 | PorARc | 2 | E78R/D82S/E89F/ | DNA squiggle around open pore | 6 |
| 9324 | | | E116T/E125A | level. Increased median current. | |
| | | | D165S | Increase in SNR due to increase in | |
| | | | | range. | |
| | | | | Compared to ONLP18393. | |
| ONLP1 | PorARc | 2 | E78R/D82S/E89S/ | DNA squiggle around open pore | 7 |
| 9328 | | | E116T/E125A/ | level. Increased median current. | |
| | | | D165S | Increase in SNR due to increase in | |
| | | | | range. | |
| | | | | Compared to ONLP18393. | |
| ONLP1 | PorARc | 2 | E78R/D82S/E89T/ | DNA squiggle around open pore | 7 |
| 9327 | | | E116T/E125A/ | level. Increased median current. | |
| | | | D165S | Increase in SNR due to increase in | |
| | | | | range. | |
| | | | | Compared to ONLP18393. | |
| ONLP1 | PorARc | 2 | E78R/D82S/E89Q/ | DNA squiggle around open pore | 7 |
| 9326 | | | E116T/E125A/ | level. Increased median current. | |
| | | | D165S | Increase in SNR due to increase in | |
| | | | | range. | |
| | | | | Compared to ONLP18393. | |
| ONLP1 | PorARc | 2 | E78R/D82S/E89K/ | DNA squiggle well below open | 8 |
| 9537 | | | E116T/E125A/ | pore level. Similar SNR with an | |
| | | | D165S | increase in range and median sd. | |
| | | | | Compared to ONLP18393. | |
| ONLP1 | PorARc | 2 | E78R/D82S/E89R/ | Reduced open pore level. Increase | 9 |
| 9405 | | | T101V/E116T/ | in SNR due to increase in range. | |
| | | | E125A/D165S | Compared to ONLP18393. | |
| ONLP1 | PorARc | 2 | E78R/D82S/E89R/ | Increase in SNR due to increase in | 9 |
| 9404 | | | T101S/E116T/ | range. | |
| | | | E125A/D165S | Compared to ONLP18393. | |
| ONLP1 | PorARc | 2 | E78R/D82S/E89R/ | Increased open pore level and | 9 |
| 9482 | | | T101N/E116T/ | median current. Reduction in SNR | |
| | | | E125A/D165S | with an increase in range and | |
| | | | | median sd. | |
| | | | | Compared to ONLP18393. | |
| ONLP1 | PorARc | 2 | E78R/D82S/E89R/ | Increased median current. Increase | 10 |
| 9483 | | | T101Q/E116T/ | in SNR due to reduction in noise. | |
| | | | E125A/D165S | Compared to ONLP18393. | |
| ONLP1 | PorARc | 2 | E78R/D82S/E89R/ | Reduced median current. Large | 10 |
| 9481 | | | T101R/E116T/ | increase in SNR due to large | |
| | | | E125A/D165S | increase in range. | |
| | | | | Compared to ONLP18393. | |
| ONLP1 | PorARc | 2 | E78R/D82S/E89N/ | DNA squiggle around open pore | 11 |
| 9536 | | | T101V/E116T/ | level. Reduction in SNR due to a | |
| | | | E125A/D165S | reduction in range and increase in | |
| | | | | median sd. | |
| | | | | Compared to ONLP18342 | |
| ONLP1 | PorARc | 2 | E78R/D82S/E89N/ | DNA squiggle around open pore | 11 |
| 9535 | | | T101S/E116T/ | level. Increase in SNR due to | |
| | | | E125A/D165S | increase in range and reduction in | |
| | | | | median sd. | |
| | | | | Compared to ONLP19536. | |
| ONLP1 | PorARc | 2 | E78R/D82S/E89N/ | DNA squiggle around open pore | 11 |
| 9533 | | | T101N/E116T/ | level. Slight reduction in SNR | |
| | | | E125A/D165S | with a reduction in range and | |
| | | | | median sd. | |
| | | | | Compared to ONLP19536. | |
| ONLP1 | PorARc | 2 | E78R/D82S/E89N/ | DNA squiggle around open pore | 12 |
| 9534 | | | T101Q/E116T/ | level. Slight reduction in SNR | |
| | | | E125A/D165S | with an increase in range and | |
| | | | | median sd. | |
| | | | | Compared to ONLP19536. | |
| ONLP1 | PorARc | 2 | E78R/D82S/E89N/ | DNA squiggle well below open | 12 |
| 9539 | | | T101R/E116T/ | pore level with a reduction in | |
| | | | E125A/D165S | median current. Effect is similar to | |
| | | | | introducing a positive charge at | |
| | | | | the E89 position. Increase in SNR | |
| | | | | with a reduction in range and | |
| | | | | median sd. | |
| | | | | Compared to ONLP19536. | |
| ONLP1 | PorARc | 2 | E78R/D82S/G87S/ | DNA squiggle well below open | 13 |
| 9283 | | | E89R/E116T/E125A/ | pore level. Similar SNR with an | |
| | | | D165S | increase in range and median sd. | |
| | | | | Compared to ONLP18393. | |
| ONLP1 | PorARc | 2 | E78R/D82S/E89R/ | Comparable to ONLP18393. | 13 |
| 9282 | | | G91S/E116T/E125A/ | | |
| | | | D165S | | |
| ONLP1 | PorARc | 2 | E78R/D82S/E89R/ | Increase in SNR with an increase | 13 |
| 9019 | | | G91N/E116T/E125A/ | in range and median sd. | |
| | | | D165S | Compared to ONLP18393. | |
| ONLP1 | PorARc | 2 | E78R/D82S/E89R/ | Reduction in SNR due to an | 14 |
| 9018 | | | G91N/192N/E116T/ | increase in median sd. | |
| | | | E125A/D165S | Compared to ONLP18393. | |
| ONLP1 | PorARc | 2 | E78R/D82S/E89R/ | Similar SNR with a reduction in | 14 |
| 9020 | | | I92N/E116T/E125A/ | range and median sd. | |
| | | | D165S | Compared to ONLP18393. | |
| ONLP1 | PorARc | 2 | E78R/D82S/E89R/ | Similar SNR with a reduction in | 14 |
| 9281 | | | S99N/E116T/E125A/ | range and median sd. | |
| | | | D165S | Compared to ONLP18393. | |
| ONLP1 | PorARc | 2 | E78R/D82S/E89R/ | Similar SNR with similar range | 15 |
| 9280 | | | S103N/E116T/ | and median sd. | |
| | | | E125A/D165S | Compared to ONLP18393. | |
| ONLP1 | PorARc | 2 | E78R/D82S/E89R/ | Down spikes in squiggle. Reduced | 15 |
| 9538 | | | D104S/E116T/ | SNR with a large increase in range | |
| | | | E125A/D165S | and median sd. | |
| | | | | Compared to ONLP18393. | |
| ONLP1 | PorARc | 2 | E78R/D82S/E89R/ | Similar SNR with an increase in | 15 |
| 9279 | | | E116T/E122S/ | range and median sd. | |
| | | | E125A/D165S | Compared to ONLP18393. | |
| ONLP1 | PorARc | 2 | E78G/D82S/E89L/ | Reduction in median current. | 16 |
| 9074 | | | G91N/192N/E116 | DNA squiggle around open pore | |
| | | | G/E125A/D165S | level. Similar SNR with an | |
| | | | | increase in range and median sd. | |
| | | | | Compared to ONLP19323. | |
| ONLP1 | PorARc | 2 | E78G/D82S/E89L/ | Reduction in median current. | 16 |
| 9075 | | | E116G/E125A/ | DNA squiggle around open pore | |
| | | | D165S | level. Similar SNR with an | |
| | | | | increase in range and median sd. | |
| | | | | Compared to ONLP19323. | |
| ONLP1 | PorARr | 4 | E182R/E184R | Clear squiggle observed. Increase | 17 |
| 9675 | | | | in SNR with reduction in range | |
| | | | | and median sd. | |
| | | | | Compared to majority of PorARc | |
| | | | | mutants. | |
| ONLP1 | PorARr | 4 | E89N/E91N | Clear squiggle observed. Median | 17 |
| 9676 | | | | current of the squiggle is higher | |
| | | | | than ONLP19675. | |
| ONLP1 | PorARr | 4 | E89N/E91N/E168 | Clear squiggle observed. Median | 17 |
| 9677 | | | R/E182R/E184R | current of the squiggle is much | |
| | | | | lower than the open pore level. | |
| ONLP1 | PorARr | 4 | E89N/E91N/E96R/ | Clear squiggle observed. | 18 |
| 9678 | | | E99N/E113N/E12 | | |
| | | | 3N/D130N/E182R/ | | |
| | | | E184R | | |
| ONLP1 | PorARr | 4 | E89N/E91N/E96R/ | Higher median current of the | 18 |
| 9679 | | | E99R/E113K/E123K/ | DNA squiggle. Reduction in SNR | |
| | | | D130K/E182R/ | due to reduction in range. | |
| | | | E184R | Compared to ONLP19675. | |
| ONLP1 | PorBRr | 6 | — | Unclear if pore can capture DNA. | 19 |
| 9635 | | | | | |
| ONLP1 | PorBRr | 6 | E90N | Possible DNA squiggle observed. | 19 |
| 9636 | | | | | |
| ONLP1 | PorBRr | 6 | E92N | Possible DNA squiggle observed. | 19 |
| 9637 | | | | | |
| ONLP1 | PorBRr | 6 | E90N/E92N | Clear DNA squiggle observed. | 20 |
| 9638 | | | | Visually nice squiggle. Increase in | |
| | | | | SNR due to a reduction in median | |
| | | | | sd. | |
| | | | | Compared to all PorARc mutants. | |
| ONLP1 | PorBRr | 6 | E90N/E92N/E115R/ | Clear DNA squiggle observed. | 20 |
| 9639 | | | E131R | Visually nice squiggle. Increased | |
| | | | | DNA capture. Increase in open | |
| | | | | pore current and median current of | |
| | | | | the DNA squiggle. Reduction in | |
| | | | | SNR with an increase in range and | |
| | | | | median sd. | |
| | | | | Compared to ONLP19638. | |
| ONLP1 | PorBRr | 6 | E90N/E92N/E113 | Clear DNA squiggle observed. | 20 |
| 9640 | | | R/E115R/E129R/ | Visually nice squiggle. Increased | |
| | | | E131R | SNR, with reduced range and | |
| | | | | median sd. | |
| | | | | Compared to ONLP19639. | |

### Example 2—Additional PorBr Data

### Methods

E coli Pore Production

DNA encoding the mature forms of the PorBRr proteins (SEQ ID NO: 6 with the substitutions shown in Table 4 below) were synthesized by GENEWIZ Germany GmbH and Twist Bioscience plc. USA and cloned into a pT7 vector containing ampicillin resistance gene. DNA concentration was adjusted to 400 ng/μL.

The plasmid DNA was thawed at room temperature and mixed by slowly pipetting up and down. Chemically competent Lemo21(DE3) E. coli cells were thawed on ice. 1 μl of DNA at 400 ng/l was added to the cells and mixed by flicking and agitating the tube. This was then left on ice for 30 minutes before heat shocking the cells at 42° C. for 10 seconds. The cells were then left on ice for 5 minutes. 150 μl of SOC (Sigma, S1797) media that was pre-warmed to 37° C. was added to the cells and left for one hour at 37° C. with shaking at 250 rpm. 75 ul of outgrown cell culture was then plated out on a big LB agar plate containing 100 μg/ml ampicillin, 41 μg/ml chloramphenicol and 0.2% glucose and then left to incubate overnight at 37° C.

A single colony of the transformed Lemo21 (DE3) cells was picked and inoculated in 100 ml LB medium with 100 μg/ml ampicillin, 41 μg/ml chloramphenicol and 0.2% glucose. This starter culture was incubated overnight at 37° C. and 250 rpm in a 500 ml flask. Main culture of 500 ml LB medium containing 100 μg/ml ampicillin, 41 μg/ml chloramphenicol and 0.2% glucose were set up in 2.5 l flask. This was then added spiked with 4 ml of starter culture (dilution 1:125) and the cells were left to divide at 37° C. and 250 rpm until O.D 0.6 was reached. Upon reaching O.D. 0.6, the temperature of the incubator was reduced to 18° C., and the cells were induced with 0.2 mM IPTG (final concentration in the medium). The cells were incubated overnight at 18° C. and 250 rpm to allow overexpression of the protein of interest. Finally, the cells were harvested by spinning them at 6000 g for 20 min at 4° C.

The cell paste was weighed to calculate the right volume of functional lysis buffer to prepare (cells are to be resuspended in 100 ml lysis buffer per 10 g of paste). The required amount of functional lysis buffer was prepared by adding benzonase (10 μl/100 ml, Sigma), tablets of EDTA-free protease inhibitor cocktail (1 tablet/100 ml, Sigma) and 10× BugBuster Protein Extraction Reagent (10 ml/100 ml, Sigma) to buffer containing 50 mM Tris/HCl, 150 mM NaCl, 2 mM EDTA, 0.1% DDM. pH of resulting functional lysis buffer was then adjusted to pH 8 at room temperature. The cells were resuspended in functional lysis buffer and mixed for 1 hour at room temperature with a magnetic stirrer to result in homogenous mixture. The cells were then lysed by sonication on ice (Soniprep150, 10 cycles 10 s on/20s off, 10 μm amplitude) and left to solubilize for 3.5h stirring at RT. The cell extract was then transferred to 100 ml Beckman tubes and centrifuged at 40,000 g for 35 minutes at room temperature. Resulting supernatant was then filtered using sterile 0.2 μm vacuum filter (Nalgene Rapid-Flow, Sigma)

### Affinity Chromatography

Filtered supernatant was loaded onto a 5 mL StrepTrap HP column (Fisher, 10571680). The column was washed with 20 column volumes (CV) of Mobile phase A (25 mM Tris, 150 mM NaCl, 2 mM EDTA 0.1% DDM, pH 8.0). The column was then washed with 7 CV of 25 mM Tris, 2 M NaCl, 2 mM EDTA, 0.1% DDM, pH 8.0 buffer before being returned back to Mobile phase A. Elution was carried out by flowing 7 CV of Mobile phase B buffer (25 mM Tris, 150 mM NaCl, 2 mM EDTA 0.1% DDM, 10 mM Desthiobiotin pH 8.0) through the column.

### Anion Exchange Chromatography

Resulting fractions containing Affinity chromatography eluate were pooled together and diluted 5× with AEX Mobile Phase A (25 mM Tris, 0.1% DDM, pH 9.0) to reduce the salt concentration in the sample. The resulting Strep purified sample was then subsequently loaded onto POROS HQ10 2 ml column (ThermoFisher). The column was then washed with 20 CV of AEX Mobile phase A before elution with 2M NaCl over a gradient of 0-50% over 35 CV, where AEX Mobile phase B comprised of 25 mM Tris, 2M NaCl, 0.1% DDM, pH 9.0.

Fractions of interest were screened using non-denaturing Native-PAGE. The fractions that contained the protein of interest in the right oligomeric state were pooled together and used in electrophysiological recordings.

### Electrophysiology Data

Electrical measurements were acquired from PorBRr mutant nanopores that were inserted into MinION flow cells. After a single pore inserted into the block co-polymer membrane, 1 mL of a buffer comprising 25 mM Potassium Phosphate, 150 mM Potassium Ferrocyanide (II), 150 mM Potassium Ferricyanide (III), pH 8.0 was flowed through the system to remove any excess protein nanopores.

The analyte being used to assess the DNA squiggle was a 3.6-kilobase ssDNA section from the 3′ end of the lambda genome. Preparation of the analyte, ligating the analyte to the Y-adapter, SPRI-bead clean-up of the ligated analyte and addition to a MinION flow cell was carried out using the Oxford Nanopore Technologies Q-SQK-LSK109 protocol.

Electrical measurements were acquired using MinION Mk1b from Oxford Nanopore Technologies. A standard sequencing script at −180 mV was run for 1-6 hours, with static flicks (voltage reversal to +100 mV) every 5 minutes to remove extended nanopore blocks. Raw data was collected in a bulk FAST5 file using MinKNOW software (Oxford Nanopore Technologies). Collected data was filtered to the small population of protein channels given the signal metrics were better than from other pore populations present. The median number of strands was 6000 across all pore mutants. The results are shown in FIG. 22 and Table 4 below. In both FIG. 22 and Table 4, PorBRr-WT-E90N-E92N-E115R-E131R_ONLZ16955 (bold in Table 4) can be considered to be a baseline. All of the mutants in Table 4 are improved in relation to one or more of SNR, range, median SD and normalised MAD current compared with PorBRr-WT-E90N-E92N-E115R-E131R_ONLZ16955.

TABLE 4
Summary of data in FIG. 22

| | | | | Normalised |
|---|---|---|---|---|
| | | | Median | MAD |
| Mutant | SNR | Range/pA | SD/pA | current |
| PorBRr-WT-E90G-E92N- | 17.589 | 17.807 | 1.012 | 0.402 |
| E115R-E131R_ONLZ18535 | | | | |
| PorBRr-WT-E90A-E92N- | 16.711 | 17.340 | 1.038 | 0.400 |
| E115R-E131R_ONLZ18536 | | | | |
| PorBRr-WT-E90V-E92N- | 16.837 | 17.909 | 1.064 | 0.401 |
| E115R-E131R_ONLZ18537 | | | | |
| PorBRr-WT-E90L-E92N- | 17.311 | 18.131 | 1.047 | 0.399 |
| E115R-E131R_ONLZ18538 | | | | |
| PorBRr-WT-E90I-E92N- | 16.520 | 18.559 | 1.123 | 0.401 |
| E115R-E131R_ONLZ18539 | | | | |
| PorBRr-WT-E90F-E92N- | 15.808 | 17.902 | 1.132 | 0.399 |
| E115R-E131R_ONLZ18540 | | | | |
| PorBRr-WT-E90Y-E92N- | 16.946 | 17.122 | 1.010 | 0.400 |
| E115R-E131R_ONLZ18541 | | | | |
| PorBRr-WT-E90S-E92N- | 16.649 | 17.790 | 1.069 | 0.398 |
| E115R-E131R_ONLZ18532 | | | | |
| PorBRr-WT-E90T-E92N- | 16.837 | 18.364 | 1.091 | 0.400 |
| E115R-E131R_ONLZ18533 | | | | |
| PorBRr-WT-E90Q-E92N- | 16.770 | 18.203 | 1.085 | 0.400 |
| E115R-E131R_ONLZ18534 | | | | |
| PorBRr-WT-E90N-E92G- | 17.110 | 17.412 | 1.018 | 0.409 |
| E115R-E131R_ONLZ18547 | | | | |
| PorBRr-WT-E90N-E92A- | 19.711 | 18.541 | 0.941 | 0.415 |
| E115R-E131R_ONLZ18548 | | | | |
| PorBRr-WT-E90N-E92V- | 19.045 | 17.212 | 0.904 | 0.406 |
| E115R-E131R_ONLZ18549 | | | | |
| PorBRr-WT-E90N-E92L- | 18.107 | 17.740 | 0.980 | 0.408 |
| E115R-E131R_ONLZ18550 | | | | |
| PorBRr-WT-E90N-E92I- | 19.157 | 17.094 | 0.892 | 0.407 |
| E115R-E131R_ONLZ18551 | | | | |
| PorBRr-WT-E90N-E92F- | 14.578 | 17.290 | 1.186 | 0.395 |
| E115R-E131R_ONLZ18552 | | | | |
| PorBRr-WT-E90N-E92Y- | 13.640 | 16.430 | 1.205 | 0.401 |
| E115R-E131R_ONLZ18553 | | | | |
| PorBRr-WT-E90N-E92S- | 17.149 | 18.626 | 1.086 | 0.412 |
| E115R-E131R_ONLZ18544 | | | | |
| PorBRr-WT-E90N-E92T- | 18.104 | 17.244 | 0.952 | 0.408 |
| E115R-E131R_ONLZ18545 | | | | |
| PorBRr-WT-E90N-E92Q- | 17.344 | 17.123 | 0.987 | 0.409 |
| E115R-E131R_ONLZ18546 | | | | |
| PorBRr-WT-E90N-E92N- | 17.883 | 18.840 | 1.054 | 0.407 |
| T100N-E115R- | | | | |
| E131R_ONLZ18634 | | | | |
| PorBRr-WT-E90N-E92N- | 16.472 | 16.679 | 1.013 | 0.406 |
| T102A-E115R- | | | | |
| E131R_ONLZ18625 | | | | |
| PorBRr-WT-E90N-E92N- | 17.615 | 16.530 | 0.938 | 0.406 |
| T102V-E115R- | | | | |
| E131R_ONLZ18626 | | | | |
| PorBRr-WT-E90N-E92N- | 17.173 | 17.054 | 0.993 | 0.403 |
| T102N-E115R- | | | | |
| E131R_ONLZ18623 | | | | |
| PorBRr-WT-E90N-E92N- | 15.953 | 15.294 | 0.959 | 0.406 |
| T102Q-E115R- | | | | |
| E131R_ONLZ18624 | | | | |
| PorBRr-WT-E90N-E92N- | 15.559 | 16.664 | 1.071 | 0.401 |
| T104A-E115R- | | | | |
| E131R_ONLZ18629 | | | | |
| PorBRr-WT-E90N-E92N- | 17.108 | 17.224 | 1.007 | 0.400 |
| T104N-E115R- | | | | |
| E131R_ONLZ18627 | | | | |
| PorBRr-WT-E90N-E92N- | 17.989 | 17.103 | 0.951 | 0.403 |
| T102V-T104N-E115R- | | | | |
| E131R_ONLZ18633 | | | | |
| PorBRr-WT-E90N-E92N- | 17.045 | 17.793 | 1.044 | 0.398 |
| T102N-T104N-E115R- | | | | |
| E131R_ONLZ18631 | | | | |
| PorBRr-WT-E90N-E92N- | 15.378 | 15.159 | 0.986 | 0.399 |
| D105A-E115R- | | | | |
| E131R_ONLZ18640 | | | | |
| PorBRr-WT-E90N-E92N- | 15.739 | 16.124 | 1.024 | 0.402 |
| E113R-E115R-E129R- | | | | |
| E131R_ONLZ16956 | | | | |
| PorBRr-WT-E90N-E92N- | 16.502 | 16.559 | 1.003 | 0.399 |
| E115R-E131R_ONLZ16955 | | | | |

### SEQUENCE LISTING

SEQ ID NO: 1
catatggcggttgatgatagcaacagcgttgtggatggtggcggtaacacgatcaccgtgagtcagagcgacaccttcat
caacagcgtgtttccgctggatggtagcccactgacccgcgaatggtttcataacggtcgcgccatcgttgatgtgaccg
gtccggatgccgaagatttcagcggcaccgtgaccatcggctaccaagttggctatccagccagtctgggtggcgaactg
acctttgactacaccaccccgggtctggaactgggtatcagcagcgcgggtgccgttagtggcacgctgagcgatgttct
gccacaagccggcgttggcgttacgctggaaccgggcccgggtatcgaaaccgttgaagttgcgagtggtgcggccagtg
gcgcgcataccgaaatccagatcgccaatctgcacggcaccgcgacgaaaatcgccggcaatgttagcgtgcgcccatac
gtgcaagttgttagcgataatggcgatgtggcgaccacctttggtcagccttggcggttcaatggtagtggtggtgagaa
tctgtattttcagggttctggtagtggttctgcgtggtcacatcctcaattcgagaaataatga
SEQ ID NO: 2
MAVDDSNSVVDGGGNTITVSQSDTFINSVFPLDGSPLTREWFHNGRAIVDVTGPDAEDF
SGTVTIGYQVGYPASLGGELTFDYTTPGLELGISSAGAVSGTLSDVLPQAGVGVTLEPGP
GIETVEVASGAASGAHTEIQIANLHGTATKIAGNVSVRPYVQVVSDNGDVATTFGQPWR
FNGSGGENLYFQGSGSGSAWSHPQFEK
SEQ ID NO: 3
catatggtggtagatgatcaaaatcgtattgtctcagacggccatgaagtaatcgtaactcaagaagatacttttatcca
gggagttccgccaattggtggatcacctctttcgcgcgagtggttccataacggacgtgggatcgctaatattgtagggc
cggatgccgcagatttcgagggatcgacgttccaatttgggtatcaatttgcctggagcggcagtttagatgggagcatt
ggggtcgcatggtcttctccgcaggccgaacttgaagttgctccgggggaagtctatgaacccgccaccgagactaagac
agtagacgggaaagagacggaggtccccgtcacagctccggttttgaatgaggatggctcgcctattttggacgccgacg
ggaaaccagttactgagcagctttatcgtatcaagccggacgcaacggcgacggagaatacgttgacggttggaggttta
ttaccacaacttacagctggcattgagcttactcccgctcccggcattgaggaacttgtggtggctgagggcgaatttga
cggcgatttcaaatctgttcaaatcgccaatgtacacggagcggccacaggtgtcattggagctgtgagtgtccgcccct
ttgtgcgtgtcattactgccaacggggataatgtaactacttatggtaaggtttggacgctgggctctgggggagagaat
ctttacttccaaggctccggcagcggttccgcttggtcacatcctcagtttgagaaa
SEQ ID NO: 4
MVVDDQNRIVSDGHEVIVTQEDTFIQGVPPIGGSPLSREWFHNGRGIANIVGPDAADFEG
STFQFGYQFAWSGSLDGSIGVAWSSPQAELEVAPGEVYEPATETKTVDGKETEVPVTAP
VLNEDGSPILDADGKPVTEQLYRIKPDATATENTLTVGGLLPQLTAGIELTPAPGIEELVV
AEGEFDGDFKSVQIANVHGAATGVIGAVSVRPFVRVITANGDNVTTYGKVWTLGSGGE
NLYFQGSGSGSAWSHPQFEK
SEQ ID NO: 5
catatggctgtggacgaccagaatcgcatcgtgagcaccgatggttacgaggtggtggttacccaagaagatacgttcat
ccaaggcgttccagcgctgggtggtagtccgttcaaccgcgagttcttccacaatggtcgcggcaccgccaatctggttg
gtgccgatgccgcggatgccgaaggtaccacctttcagttcggctaccagtttgcgtggggggtagcattgatggcgcga
tcggcgttacctatagcaccccgggtctggaactggaactgagcgccaaagatggcccgaccgcgaccgttaccgatatt
ctgccacaagcctatgcggaactcgaactgaccccagcgccgggcatcgaagaactggtggttgcggagggtgaattcga
cggcgatttcaagagcgtgcagttcagcaacgtgcatggtaccgccagcggtgttctgggtgcggttcaagttcgcccat
ttgtgcgcgcgatcaccgcgaatggcgacaacgttacaacctatggcaaaccttggaccgttggtagtggtggtgagaat
ctgtattttcagggttctggtagtggttctgcgtggtcacatcctcaattcgagaaataatga
SEQ ID NO: 6
MAVDDQNRIVSTDGYEVVVTQEDTFIQGVPALGGSPFNREFFHNGRGTANLVGADAAD
AEGTTFQFGYQFAWAGSIDGAIGVTYSTPGLELELSAKDGPTATVTDILPQAYAELELTP
APGIEELVVAEGEFDGDFKSVQFSNVHGTASGVLGAVQVRPFVRAITANGDNVTTYGKP
WTVGSGGENLYFQGSGSGSAWSHPQFEK

1. A mutant Rhodococcus pore comprising a monomer which is a variant of the sequence shown in SEQ ID NO: 2, wherein the variant comprises one or more mutations at one or more of the following positions:
(a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
(b) S74, G76, E78, T80, D82, T84, Q108, G110, G112, T114, E116, and P117; and/or
(c) G87, E89, G91, S93, S94, A95, G96, A97, S99, T101, S103, and D104;
wherein the one or more mutations alter the ability of the monomer to interact with a target analyte.

2. A mutant pore according to claim 1, wherein the variant comprises one or more mutations at one or more of the positions in (a), (b), (c), (a) and (b), (b) and (c), (a) and (c), or (a), (b) and (c).

3. A mutant pore according to claim 1 or 2 wherein the variant comprises one or more mutations at least at the following positions:
E78/D82/E89,
E78/D82/E89/E116/E125/D165,
E57/E78/D82/E89/E116/E125/D165,
D55/E57/D58/E78/D82/E89/E116/E125/D165,
E78/D82/E89/D104/E116/E122/E125/D165,
D55/E57/D58/E78/D82/E89/D104/E116/E122/E125/D165,
E57/E78/D82/E89/D104/E116/E122/E125/D165,
E78/D82/E89/T101/E116/E125/D165,
E78/D82/G87/E89/E116/E125/D165,
E78/D82/E89/G91/I92/E116/E125/D165,
E78/D82/E89/I92/E116/E125/D165,
E78/D82/E89/S99/E116/E125/D165,
E78/D82/E89/S103/E116/E125/D165,
E78/D82/E89/D104/E116/E125/D165,
E78/D82/E89/E116/E122/E125/D165,
E78/D82/E89/G91/I92/E116/E125/D165, or
E78/D82/E89/E116/E125/D165.

4. A mutant pore according to any one of the preceding claims, wherein the one or more mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position.

5. A mutant pore according to any one of the preceding claims, wherein the one or more mutations are one or more substitutions.

6. A mutant pore according to any one of the preceding claims, wherein the one or more mutations are selected from:
D to Q, S or Y, more preferably D to S,
G to S, N, Q, T or A, more preferably G to S or N,
N to S, Q, L, Y, M, I, V or G,
T to V, S, N, Q, R, F or I, more preferably T to V, S, N, Q or R,
P to S,
A to R, V, D, N, S or T,
E to S, N, T, A, R, G, V, L, I, F, Q or K,
F to R, K, S, T, N, Q, G, A, V, L, I, P, C, Y or W,
S to N or C, more preferably S to N,
Y to R, K, S, T, N, Q, G, A, V, L, I, P, C, D, E, F, or W,
Q to R,
V to I,
I to G, L, S, F, V, A, P, W, Q, N, C, S, Y, K or T, more preferably I to N,
E to R,
H to F, N, Q or A,
L to N, S, Y, V, Q, T, R, A, G, C, F or D, and
K to R, S, T, N, Q, G, A, V, L, I, P, C, D, E, F, Y or W.

7. A mutant pore according to any one of the preceding claims, wherein the variant comprises at least the following mutations:
E78S/D82S/E89N,
E78S/D82S/E89N/E116T/E125A/D165S,
E78S/D82S/E89R,
E78R/D82S/E89R,
E78R/D82S/E89R/E116T/E125A/D165S,
E57R/E78R/D82S/E89R/E116T/E125A/D165S,
D55S/E57S/D58S/E78R/D82S/E89R/E116T/E125A/D165S,
E78R/D82S/E89R/D104S/E116T/E122S/E125A/D165S,
D55S/E57S/D58S/E78R/D82S/E89R/D104S/E116T/E122S/E125A/D165S,
E57R/E78R/D82S/E89R/D104S/E116T/E122S/E125A/D165S,
E78R/D82S/E89G/E116T/E125A/D165S,
E78R/D82S/E89A/E116T/E125A/D165S,
E78R/D82S/E89V/E116T/E125A/D165S,
E78R/D82S/E89L/E116T/E125A/D165S,
E78R/D82S/E89I/E116T/E125A/D165S,
E78R/D82S/E89F/E116T/E125A/D165S,
E78R/D82S/E89S/E116T/E125A/D165S,
E78R/D82S/E89T/E116T/E125A/D165S,
E78R/D82S/E89Q/E116T/E125A/D165S,
E78R/D82S/E89K/E116T/E125A/D165S,
E78R/D82S/E89R/T101V/E116T/E125A/D165S,
E78R/D82S/E89R/T101S/E116T/E125A/D165S,
E78R/D82S/E89R/T101N/E116T/E125A/D165S,
E78R/D82S/E89R/T101Q/E116T/E125A/D165S,
E78R/D82S/E89R/T101R/E116T/E125A/D165S,
E78R/D82S/E89N/T101V/E116T/E125A/D165S,
E78R/D82S/E89N/T101S/E116T/E125A/D165S,
E78R/D82S/E89N/T101N/E116T/E125A/D165S,
E78R/D82S/E89N/T101Q/E116T/E125A/D165S,
E78R/D82S/E89N/T101R/E116T/E125A/D165S,
E78R/D82S/G87S/E89R/E116T/E125A/D165S,
E78R/D82S/E89R/G91S/E116T/E125A/D165S,
E78R/D82S/E89R/G91N/E116T/E125A/D165S,
E78R/D82S/E89R/G91N/I92N/E116T/E125A/D165S,
E78R/D82S/E89R/I92N/E116T/E125A/D165S,
E78R/D82S/E89R/S99N/E116T/E125A/D165S,
E78R/D82S/E89R/S103N/E116T/E125A/D165S,
E78R/D82S/E89R/D104S/E116T/E125A/D165S,
E78R/D82S/E89R/E116T/E122S/E125A/D165S,
E78G/D82S/E89L/G91N/I92N/E116G/E125A/D165S, or
E78G/D82S/E89L/E116G/E125A/D165S.

8. A mutant Rhodococcus pore comprising a monomer which is a variant of the sequence shown in SEQ ID NO: 6, wherein the variant comprises one or more mutations between positions 9 and 200 of SEQ ID NO: 6 and wherein the one or more mutations alter the ability of the pore to interact with a target analyte.

9. A mutant pore according to any one of the preceding claims, wherein the variant has at least 70% identity to the sequence shown in SEQ ID NO: 2.

10. A mutant pore according to any one of the preceding claims, wherein the mutant is chemically modified.

11. A mutant pore according to claim 10, wherein the mutant is chemically modified by attachment of a molecule to one or more cysteines, attachment of a molecule to one or more lysines, attachment of a molecule to one or more non-natural amino acids, enzyme modification of an epitope or modification of a terminus.

12. A mutant pore according to claim 11, wherein the one or more cysteines have been introduced to the mutant by substitution.

13. A mutant pore according to claim 11 or 12, wherein the molecule is (a) a molecular adaptor that facilitates the interaction between the monomer and a target analyte or (b) a polynucleotide binding protein.

14. A construct comprising two or more covalently attached Rhodococcus porin monomers.

15. A construct according to claim 14, wherein at least one of the monomers is a variant as defined in any one of claims 1-13.

16. A construct according to claim 14 or 15, wherein the monomers are genetically fused and/or are attached via a linker.

17. A polynucleotide which encodes a mutant pore according to any one of claims 1-13 or a construct according to any one of claims 14-16.

18. A pore comprising at least one construct according to any one of claims 14-16.

19. A kit for characterising a target polynucleotide or a target polypeptide comprising (a) a pore according to any one of claims 1-13 and 18 and (b) a polynucleotide binding protein or a polypeptide handling enzyme.

20. An apparatus for characterising a target polynucleotide or a target polypeptide in a sample, comprising (a) a plurality of pores according to any one of claims 1-13 and 18 and (b) a plurality of polynucleotide binding proteins or a plurality of polypeptide handling enzymes.

21. A membrane comprising a pore according to any one of claims 1-13 and 18.

22. An array comprising a plurality of membranes according to claim 21.

23. A system comprising (a) a membrane according to claim 21 or an array according to claim 22, (b) means for applying a potential across the membrane(s) and (c) means for detecting electrical or optical signals across the membrane(s).

24. A method of determining the presence, absence or one or more characteristics of a target analyte, comprising (a) contacting the target analyte with a pore according to any one of claims 1-13 and 18 and (b) taking one or more measurements as the target analyte moves with respect to the pore and thereby determining the presence, absence or one or more characteristics of the target analyte.

25. A method according to claim 24, wherein the target analyte is or comprises a polypeptide, a polynucleotide, or a polysaccharide.

26. A method according to claim 24 or 25, wherein the target analyte comprises a polynucleotide and said method comprises (i) contacting the polynucleotide with a polynucleotide binding protein capable of controlling the movement of the polynucleotide with respect to the pore; and (ii) taking one or more measurements characteristic of the polynucleotide as the polynucleotide moves with respect to the pore.

27. A method according to claim 24 or 25, wherein the target analyte comprises a polypeptide and said method comprises (i) contacting the polypeptide with a polypeptide handling enzyme capable of controlling the movement of the polypeptide with respect to the pore; and (ii) taking one or more measurements characteristic of the polypeptide as the polypeptide moves with respect to the pore.

28. A method according to claim 24 or 25, wherein the target analyte comprises a polynucleotide-polypeptide conjugate and said method comprises (i) contacting the conjugate with a polynucleotide binding protein capable of controlling the movement of the polynucleotide of the conjugate with respect to the pore; and (ii) taking one or more measurements characteristic of the polypeptide as the conjugate moves with respect to the pore.

29. A method of characterising a target polynucleotide, comprising (a) contacting the target polynucleotide with a pore according to any one of claims 1-13 and 18 and (b) taking one or more measurements as the target polynucleotide moves with respect to the pore and thereby characterising the target polynucleotide.

30. Use of a pore according to any one of claims 1-13 and 18 to determine the presence, absence or one or more characteristics of a target analyte or target polynucleotide.

31. An apparatus comprising a transmembrane protein pore inserted into an in vitro membrane, wherein the transmembrane protein pore comprises at least one monomer which is a variant of the Rhodococcus porin monomer and comprising mutations at one or more of the following positions:
(a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
(b) S74, G76, E78, T80, D82, T84, Q108, G110, G112, T114, E116, and P117; and/or
(c) G87, E89, G91, S93, S94, A95, G96, A97, S99, T101, S103, and D104, corresponding to the sequence shown in SEQ ID NO: 2.

32. An apparatus produced by a method comprising:
(i) obtaining a transmembrane protein pore comprising at least one Rhodococcus porin monomer, wherein the monomer comprises amino acid mutations at one or more of positions
(a) D11, G12, G13, G14, N15, T52, G53, P54, D55, A56, E57, D58, F59, S60, G61, T62, T64, Y67, Q68, V69, Y71, P119, G120, I121, E122, T123, V124, E125, V126, S128, A130, A131, S132, G133, A134, H135, L143, H144, T146, T148, K149, Y159, Q161, V163, S164, D165, N166, G167, D168, V169, and T171;
(b) S74, G76, E78, T80, D82, T84, Q108, G110, G112, T114, E116, and P117; and/or
(c) G87, E89, G91, S93, S94, A95, G96, A97, S99, T101, S103, and D104 corresponding to the sequence shown in SEQ ID NO: 2; and
(ii) contacting the pore with an in vitro membrane such that the pore is inserted in the in vitro membrane.


SEQUENCE LISTING (6 sequences)

SEQ ID NO: 1 - DNA, 624 bp, Rhodococcus corynebacterioides
  source 1..624: mol_type = genomic DNA
catatggcggttgatgatagcaacagcgttgtggatggtggcggtaacacgatcaccgtgagtcagagcgacaccttcatcaacagcgtgtttccgctggatggtagcccactgacccgcgaatggtttcataacggtcgcgccatcgttgatgtgaccggtccggatgccgaagatttcagcggcaccgtgaccatcggctaccaagttggctatccagccagtctgggtggcgaactgacctttgactacaccaccccgggtctggaactgggtatcagcagcgcgggtgccgttagtggcacgctgagcgatgttctgccacaagccggcgttggcgttacgctggaaccgggcccgggtatcgaaaccgttgaagttgcgagtggtgcggccagtggcgcgcataccgaaatccagatcgccaatctgcacggcaccgcgacgaaaatcgccggcaatgttagcgtgcgcccatacgtgcaagttgttagcgataatggcgatgtggcgaccacctttggtcagccttggcggttcaatggtagtggtggtgagaatctgtattttcagggttctggtagtggttctgcgtggtcacatcctcaattcgagaaataatga

SEQ ID NO: 2 - protein, 205 aa, Rhodococcus corynebacterioides
MAVDDSNSVVDGGGNTITVSQSDTFINSVFPLDGSPLTREWFHNGRAIVDVTGPDAEDFSGTVTIGYQVGYPASLGGELTFDYTTPGLELGISSAGAVSGTLSDVLPQAGVGVTLEPGPGIETVEVASGAASGAHTEIQIANLHGTATKIAGNVSVRPYVQVVSDNGDVATTFGQPWRFNGSGGENLYFQGSGSGSAWSHPQFEK

SEQ ID NO: 3 - DNA, 777 bp, Rhodococcus ruber
  source 1..777: mol_type = genomic DNA
catatggtggtagatgatcaaaatcgtattgtctcagacggccatgaagtaatcgtaactcaagaagatacttttatccagggagttccgccaattggtggatcacctctttcgcgcgagtggttccataacggacgtgggatcgctaatattgtagggccggatgccgcagatttcgagggatcgacgttccaatttgggtatcaatttgcctggagcggcagtttagatgggagcattggggtcgcatggtcttctccgcaggccgaacttgaagttgctccgggggaagtctatgaacccgccaccgagactaagacagtagacgggaaagagacggaggtccccgtcacagctccggttttgaatgaggatggctcgcctattttggacgccgacgggaaaccagttactgagcagctttatcgtatcaagccggacgcaacggcgacggagaatacgttgacggttggaggtttattaccacaacttacagctggcattgagcttactcccgctcccggcattgaggaacttgtggtggctgagggcgaatttgacggcgatttcaaatctgttcaaatcgccaatgtacacggagcggccacaggtgtcattggagctgtgagtgtccgcccctttgtgcgtgtcattactgccaacggggataatgtaactacttatggtaaggtttggacgctgggctctgggggagagaatctttacttccaaggctccggcagcggttccgcttggtcacatcctcagtttgagaaa

SEQ ID NO: 4 - protein, 258 aa, Rhodococcus ruber
MVVDDQNRIVSDGHEVIVTQEDTFIQGVPPIGGSPLSREWFHNGRGIANIVGPDAADFEGSTFQFGYQFAWSGSLDGSIGVAWSSPQAELEVAPGEVYEPATETKTVDGKETEVPVTAPVLNEDGSPILDADGKPVTEQLYRIKPDATATENTLTVGGLLPQLTAGIELTPAPGIEELVVAEGEFDGDFKSVQIANVHGAATGVIGAVSVRPFVRVITANGDNVTTYGKVWTLGSGGENLYFQGSGSGSAWSHPQFEK

SEQ ID NO: 5 - DNA, 624 bp, Rhodococcus ruber
  source 1..624: mol_type = genomic DNA
catatggctgtggacgaccagaatcgcatcgtgagcaccgatggttacgaggtggtggttacccaagaagatacgttcatccaaggcgttccagcgctgggtggtagtccgttcaaccgcgagttcttccacaatggtcgcggcaccgccaatctggttggtgccgatgccgcggatgccgaaggtaccacctttcagttcggctaccagtttgcgtgggcgggtagcattgatggcgcgatcggcgttacctatagcaccccgggtctggaactggaactgagcgccaaagatggcccgaccgcgaccgttaccgatattctgccacaagcctatgcggaactcgaactgaccccagcgccgggcatcgaagaactggtggttgcggagggtgaattcgacggcgatttcaagagcgtgcagttcagcaacgtgcatggtaccgccagcggtgttctgggtgcggttcaagttcgcccatttgtgcgcgcgatcaccgcgaatggcgacaacgttacaacctatggcaaaccttggaccgttggtagtggtggtgagaatctgtattttcagggttctggtagtggttctgcgtggtcacatcctcaattcgagaaataatga

SEQ ID NO: 6 - protein, 205 aa, Rhodococcus ruber
MAVDDQNRIVSTDGYEVVVTQEDTFIQGVPALGGSPFNREFFHNGRGTANLVGADAADAEGTTFQFGYQFAWAGSIDGAIGVTYSTPGLELELSAKDGPTATVTDILPQAYAELELTPAPGIEELVVAEGEFDGDFKSVQFSNVHGTASGVLGAVQVRPFVRAITANGDNVTTYGKPWTVGSGGENLYFQGSGSGSAWSHPQFEK